(19) United States
(12) Patent Application Publication
(10) Pub. No.: US 2016/0040181 A1
(43) Pub. Date: Feb. 11, 2016

(54) AGRONOMIC CHARACTERISTICS UNDER NITROGEN LIMITING CONDITIONS FOR PLANTS EXPRESSING PH11 OR NUCPU29 POLYPEPTIDES

(71) Applicants: E I DU PONT DE NEMOURS AND COMPANY, Wilmington, DE (US); PIONEER HI-BRED INTERNATIONAL INC, Johnston, IA (US)

(72) Inventors: SANGEETA AGARWAL, Secunderabad (IN); Stephen M. Allen, Wilmington, DE (US); Milo Jay Aukerman, Newark, DE (US); Ratna Kumria, Hyderabad (IN); H. Renee Lafitte, Davis, CA (US); Stanley Luck, Wilmington, DE (US); Amitabh Mohanty, Kowkur (IN); Mark Mucha, Lincoln University, PA (US); Hajime Sakai, Newark, DE (US)

(21) Appl. No.: 14/820,738

(22) Filed: Aug. 7, 2015

Related U.S. Application Data
(60) Provisional application No. 62/076,504, filed on Nov. 7, 2014.
(60) Provisional application No. 62/034,838, filed on Aug. 8, 2014.

(51) Int. Cl. C12N 15/82

(52) U.S. Cl. CPC C12N 15/8271; C12N 15/8261; C12N 15/8255

(57) ABSTRACT

Isolated polynucleotides and polypeptides and recombinant DNA constructs particularly useful for altering agronomic characteristics of plants under nitrogen limiting conditions are described. Compositions (such as plants or seeds) comprising these recombinant DNA constructs, and methods utilizing these recombinant DNA constructs also are described. The recombinant DNA construct may comprise a polynucleotide operably linked to a heterologous promoter functional in a plant, wherein said polynucleotide encodes a PH11 or a NUCPU29 polypeptide.

### CROSS REFERENCE TO RELATED APPLICATIONS

This application claims the benefit of U.S. Provisional Application No. 62/034,838, filed Aug. 8, 2014 and U.S. Provisional Application No. 62/076,504, filed Nov. 7, 2014, the entire content of each is herein incorporated by reference.

### REFERENCE TO SEQUENCE LISTING SUBMITTED ELECTRONICALLY

The official copy of the sequence listing is submitted electronically via EFS-Web as an ASCII formatted sequence listing with a file named 20150807_BB2002USNP_SequenceListing.txt created on Aug. 7, 2015 and having a size of 212 kilobytes and is filed concurrently with the specification. The sequence listing contained in this ASCII formatted document is part of the specification and is herein incorporated by reference in its entirety.

### FIELD

The field relates to plant breeding and genetics and, in particular, relates to recombinant DNA constructs useful in plants for conferring nitrogen use efficiency and/or tolerance to nitrogen limiting conditions.

### BACKGROUND

Abiotic stressors significantly limit crop production worldwide. Cumulatively, these factors are estimated to be responsible for an average 70% reduction in agricultural production. Plants are sessile and have to adjust to the prevailing environmental conditions of their surroundings. This has led to their development of a great plasticity in gene regulation, morphogenesis, and metabolism. Adaptation and defense strategies involve the activation of genes encoding proteins important in the acclimation or defense towards the different stressors.

The adsorption of nitrogen by plants plays an important role in their growth (Gallais et al., J. Exp. Bot. 55(396):295-306 (2004)). Plants synthesize amino acids from inorganic nitrogen in the environment. Consequently, nitrogen fertilization has been a powerful tool for increasing the yield of cultivated plants, such as maize and soybean. Today farmers desire to reduce the use of nitrogen fertilizer, in order to avoid pollution by nitrates and to maintain a sufficient profit margin. If the nitrogen assimilation capacity of a plant can be increased, then increases in plant growth and yield increase are also expected. In summary, plant varieties that have better nitrogen use efficiency (NUE) are desirable.

### SUMMARY

The present disclosure includes:

In one embodiment, a plant comprising in its genome a recombinant DNA construct comprising a polynucleotide operably linked to at least one heterologous regulatory element, wherein said polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70, and wherein said plant exhibits increased nitrogen stress tolerance when compared to a control plant not comprising said recombinant DNA construct.

In another embodiment, a plant comprising in its genome a recombinant DNA construct comprising a polynucleotide operably linked to at least one heterologous regulatory element, wherein said polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70, and wherein said plant exhibits an alteration of at least one agronomic characteristic when compared to a control plant not comprising said recombinant DNA construct. Optionally, the plant exhibits said alteration of said at least one agronomic characteristic when compared, under nitrogen limiting conditions, to said control plant not comprising said recombinant DNA construct. The at least one agronomic trait may be yield, biomass, or both, and the alteration may be an increase.

In one embodiment, the polynucleotides described in the current disclosure are operably linked to a tissue-specific promoter. In one embodiment, the polynucleotides described in the current disclosure are operably linked to a promoter that is preferably expressed in at least one tissue selected from the group consisting of: root, shoot and vasculature.

In another embodiment, the present disclosure includes any of the plants of the present disclosure wherein the plant is selected from the group consisting of: maize, soybean, sunflower, sorghum, canola, wheat, alfalfa, cotton, rice, barley, millet, sugar cane and switchgrass.

In another embodiment, the present disclosure includes seed of any of the plants of the present disclosure, wherein said seed comprises in its genome a recombinant DNA construct comprising a polynucleotide operably linked to at least one heterologous regulatory element, wherein said polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70, and wherein a plant produced from said seed exhibits either an increased nitrogen stress tolerance, or an alteration of at least one agronomic characteristic, or both, when compared to a control plant not comprising said recombinant DNA construct. The at least one agronomic trait may be yield, biomass, or both and the alteration may be an increase.

In another embodiment, a method of increasing nitrogen stress tolerance in a plant, comprising (a) introducing into a regenerable plant cell a recombinant DNA construct comprising a polynucleotide operably linked to at least one heterologous regulatory sequence, wherein the polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70; (b) regenerating a transgenic plant from the regenerable plant cell after step (a), wherein the transgenic plant comprises in its genome the recombinant DNA construct; and (c) obtaining a progeny plant derived from the transgenic plant of step (b), wherein said progeny plant comprises in its genome the recombinant DNA construct and exhibits increased nitrogen stress tolerance when compared to a control plant not comprising the recombinant DNA construct.

In another embodiment, a method of selecting for increased nitrogen stress tolerance in a plant, comprising (a) obtaining a transgenic plant, wherein the transgenic plant comprises in its genome a recombinant DNA construct comprising a polynucleotide operably linked to at least one heterologous regulatory sequence, wherein the polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70; (b) growing the transgenic plant of part (a) under conditions wherein the polynucleotide is expressed; and (c) selecting for the transgenic plant of part (b) that exhibits nitrogen stress tolerance compared to a control plant not comprising the recombinant DNA construct.

In another embodiment, a method of selecting for nitrogen stress tolerance in a plant, comprising (a) introducing into a regenerable plant cell a recombinant DNA construct comprising a polynucleotide operably linked to at least one heterologous regulatory sequence, wherein the polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70; (b) regenerating a transgenic plant from the regenerable plant cell after step (a), wherein the transgenic plant comprises in its genome the recombinant DNA construct; and (c) selecting a transgenic plant of (b) that exhibits nitrogen stress tolerance compared to a control plant not comprising the recombinant DNA construct; and optionally, (d) obtaining a progeny plant derived from the transgenic plant of (c), wherein the progeny plant comprises in its genome the recombinant DNA construct; and optionally, (e) selecting a progeny plant of (d) that exhibits nitrogen stress tolerance compared to a control plant not comprising the recombinant DNA construct.

In another embodiment, a method of selecting for nitrogen stress tolerance in a plant, comprising (a) introducing into a regenerable plant cell a recombinant DNA construct comprising a polynucleotide operably linked to at least one heterologous regulatory sequence, wherein the polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70; (b) regenerating a transgenic plant from the regenerable plant cell after step (a), wherein the transgenic plant comprises in its genome the recombinant DNA construct; (c) obtaining a progeny plant derived from the transgenic plant, wherein the progeny plant comprises in its genome the recombinant DNA construct; and (d) selecting a progeny plant of (c) that exhibits nitrogen stress tolerance compared to a control plant not comprising the recombinant DNA construct.

In another embodiment, a method of selecting for altered root architecture in a plant, comprising: (a) obtaining a transgenic plant, wherein the transgenic plant comprises in its genome a recombinant DNA construct comprising a polynucleotide operably linked to at least one heterologous regulatory element, wherein said polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70; (b) obtaining a progeny plant derived from the transgenic plant, wherein the progeny plant comprises in its genome the recombinant DNA construct; and (c) selecting the progeny plant for alteration of root architecture compared to a control plant not comprising the recombinant DNA construct. The progeny plant may be compared to the control plant when grown under non-limiting nitrogen conditions.

In another embodiment, a method of selecting for an alteration of at least one agronomic characteristic in a plant, comprising (a) obtaining a transgenic plant, wherein the transgenic plant comprises in its genome a recombinant DNA construct comprising a polynucleotide operably linked to at least one heterologous regulatory sequence, wherein the polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70; (b) growing the transgenic plant of part (a) under conditions wherein the polynucleotide is expressed; and (c) selecting a transgenic plant of part (b) that exhibits an alteration of at least one agronomic characteristic when compared to a control plant not comprising the recombinant DNA construct. Optionally, said selecting step (c) comprises selecting a transgenic plant that exhibits an alteration of at least one agronomic characteristic when compared, under nitrogen limiting conditions, to a control plant not comprising the recombinant DNA construct. The at least one agronomic trait may be yield, biomass, or both and the alteration may be an increase.

In another embodiment, the present disclosure includes any of the methods of the present disclosure wherein the plant is selected from the group consisting of: maize, soybean, sunflower, sorghum, canola, wheat, alfalfa, cotton, rice, barley, millet, sugar cane and switchgrass.

In another embodiment, the present disclosure includes an isolated polynucleotide comprising: (a) a nucleotide sequence encoding a PH11 or NUCPU29 polypeptide with nitrogen stress tolerance activity, wherein the polypeptide has an amino acid sequence of at least 80%, 85%, 90%, or 95% sequence identity, based on the Clustal V method of alignment, when compared to SEQ ID NO:3, 7, 9, 11, 13, 15, 17, 19, 21, 23, 25, 27, 29, 31, 33-62 or 70, or (b) a full complement of the nucleotide sequence, wherein the full complement and the nucleotide sequence consist of the same number of nucleotides and are 100% complementary. The amino acid sequence of the PH11 or NUCPU29 polypeptide of part (a) may have less than 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70. The polypeptide may comprise the amino acid sequence of SEQ ID NO:3, 7, 9, 11, 13, 15, 17, 19, 21, 23, 25, 27, 29, 31, 33-62 or 70. The nucleotide sequence may comprise the nucleotide sequence of SEQ ID NO:2, 4, 6, 8, 10, 12, 14, 16, 18, 20, 22, 24, 26, 28, 30, 32, 69 or 71.

In another embodiment, the present disclosure concerns a recombinant DNA construct comprising any of the isolated polynucleotides of the present disclosure operably linked to at least one heterologous regulatory sequence, and a cell, a microorganism, a plant, and a seed comprising the recombinant DNA construct. The cell may be eukaryotic, e.g., a yeast, insect or plant cell, or prokaryotic, e.g., a bacterial cell.

In another embodiment, a method of producing a plant that exhibits an increase in at least one trait selected from the group consisting of: increased nitrogen stress tolerance, increased yield, increased biomass, and altered root architecture, wherein the method comprises growing a plant from a seed comprising a recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V method of alignment, using the respective default parameters, when compared to SEQ ID NO:3, 7, 9, 11, 13, 15, 17, 19, 21, 23, 25, 27, 29, 31, 33-62 or 70, wherein the plant exhibits at least one trait selected from the group consisting of: increased nitrogen stress tolerance, increased yield, increased biomass, and altered root architecture, when compared to a control plant not comprising the recombinant DNA construct.

In another embodiment, a method of producing a seed, the method comprising: (a) crossing a first plant with a second plant, wherein at least one of the first plant and the second plant comprises a recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V method of alignment, using the respective default parameters, when compared to SEQ ID NO:3, 7, 9, 11, 13, 15, 17, 19, 21, 23, 25, 27, 29, 31, 33-62 or 70; and (b) selecting a seed of the crossing of step (a), wherein the seed comprises the recombinant DNA construct. A plant grown from the seed may exhibit at least one trait selected from the group consisting of: increased nitrogen stress tolerance, increased yield, increased biomass, and altered root architecture, when compared to a control plant not comprising the recombinant DNA construct.

In another embodiment, a method of producing oil or a seed by-product, or both, from a seed, the method comprising extracting oil or a seed by-product, or both, from a seed that comprises a recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V method of alignment, using the respective default parameters, when compared to SEQ ID NO:3, 7, 9, 11, 13, 15, 17, 19, 21, 23, 25, 27, 29, 31, 33-62 or 70. The seed may be obtained from a plant that comprises the recombinant DNA construct, wherein the plant exhibits at least one trait selected from the group consisting of: increased nitrogen stress tolerance, increased yield, increased biomass, and altered root architecture, when compared to a control plant not comprising the recombinant DNA construct. The oil or the seed by-product, or both, may comprise the recombinant DNA construct.

In any of the above embodiments, the altered root architecture may be an increase in root biomass or in average root length, or both.

### BRIEF DESCRIPTION OF THE DRAWINGS AND SEQUENCE LISTING

The disclosure can be more fully understood from the following detailed description and the accompanying drawings and Sequence Listing which form a part of this application.

FIG. 1 shows a typical grid pattern for five lines (labeled 1 through 5—eleven individuals for each line), plus wild-type control C1 (nine individuals), used in screens.

FIG. 2 shows a graph of the effect of several different potassium nitrate concentrations on plant color as determined by image analysis. The response of the green color bin (hues 50 to 66) to nitrate dosage demonstrates that this bin can be used as an indicator of nitrogen assimilation.

FIG. 3 shows the growth medium used for semi-hydroponics maize growth in Example 18.

FIGS. 4A-4K show the alignment of the PH11 polypeptides from different plant species. The alignment of SEQ ID NOS. 3, 7, 9, 11, 13, 15, 17, 19, 21, 23, 25, 27, 29, 31, 33, 52-61 and 62 is shown. Residues that are identical to the residue of the AT-PH11 sequence (SEQ ID NO: 3) at a given position are enclosed in a box. A consensus or majority sequence (SEQ ID NO:63) is presented where a residue is shown if present in majority of sequences, otherwise, a period is shown.

FIG. 5 shows the percent sequence identity and the divergence values for each pair of amino acids sequences of PH11 polypeptides displayed in FIGS. 4A-4K.

FIG. 6 shows the yield analysis (in low nitrogen and normal nitrogen conditions) of transgenic maize lines comprising the recombinant construct PHP51603, overexpressing the AT-PH11 polypeptide.

FIG. 7 shows the yield analysis across locations (across all location, across all normal nitrogen locations (locations A-F), across all low nitrogen locations (locations G and H), for transgenic maize lines comprising the recombinant construct PHP51603, overexpressing the AT-PH11 polypeptide

FIG. 8 shows the yield analysis of maize lines transformed with PHP50351, expressing the Arabidopsis gene At-NUCPU29 with alternative codons (At-NUCPU29ac); under low-nitrogen (locations G-H) and normal-nitrogen conditions (locations A-F).

FIG. 9 shows the yield analysis across all locations; and across locations that are grouped according to the stress experienced in these locations.

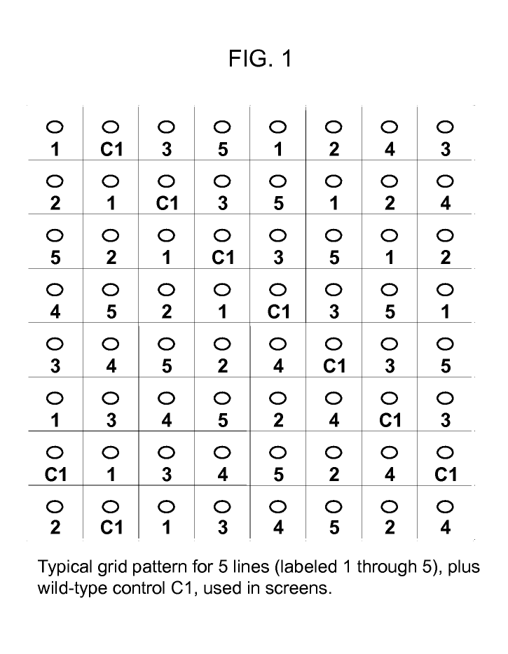

The sequence descriptions and Sequence Listing attached hereto comply with the rules governing nucleotide and/or amino acid sequence disclosures in patent applications as set forth in 37 C.F.R. §1.821-1.825. The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (2):345-373 (1984) which are herein incorporated by reference. The symbols and format used for nucleotide and amino acid sequence data comply with the rules set forth in 37 C.F.R. §1.822.

SEQ ID NO:1 is the sequence of a polylinker.

SEQ ID NO:2 is the nucleotide sequence of AT-PH11 protein-coding sequence (CDS), and corresponds to NCBI GI No. 186529090.

SEQ ID NO:3 is the amino acid sequence of the polypeptide encoded by SEQ ID NO:2, and corresponds to NCBI GI No. 15240022.

SEQ ID NO:4 is the AT-PH11 sequence with alternative codons (AT-PH11 ac).

SEQ ID NO:5 is the cDNA sequence from the locus At5g43870, encoding AT-PH11 polypeptide (Arabidopsis thaliana).

Table 1 presents SEQ ID NOs for the nucleotide sequences of the PH11 cDNA clones obtained from Zea mays, Sesbania bispinosa, Artemisia tridentata, Amaranthus hypochondriacus, Lamium amplexicaule, Delosperma nubigenum, Tradescantia sillamontana, Linum perenne, Triglochin maritima. The SEQ ID NOs for the corresponding amino acid sequences encoded by the cDNAs are also presented.

TABLE 1
cDNAs Encoding PH11 Polypeptides

| | | SEQ ID NO: | SEQ ID NO: |
|---|---|---|---|
| Plant | Clone Designation | (Nucleotide) | (Amino Acid) |
| Corn | ZM-PH11-A | 6 | 7 |
| Corn | ZM-PH11-B | 8 | 9 |
| Corn | ZM-PH11-C | 10 | 11 |
| Corn | ZM-PH11-D | 12 | 13 |
| Corn | ZM-PH11-E | 14 | 15 |
| Sesbania | sesgr1n.pk036.o8 | 16 | 17 |
| bispinosa | | | |
| Amaranthus | ahgr1c.pk088.o5 | 18 | 19 |
| hypochondriacus | | | |
| Artemisia | arttr1n.pk119.a3 | 20 | 21 |
| tridentata | | | |
| Delosperma | icegr1n.pk136.c5 | 22 | 23 |
| nubigenum | | | |
| Linum perenne | lpgr1n.pk075.f12 | 24 | 25 |
| Linum perenne | lpgr1n.pk026.f24 | 26 | 27 |
| Triglochin maritima | tmgr2n.pk006.j11 | 28 | 29 |
| Tradescantia | tsgr1n.pk037.f8 | 30 | 31 |
| sillamontana | | | |
| Tradescantia | tsgr1n.pk020.c8 | 32 | 33 |
| sillamontana | | | |

SEQ ID NO:34 is an amino acid sequence encoded by the locus At4g14740.1 of Arabidopsis thaliana.

SEQ ID NO:35 is an amino acid sequence encoded by the locus At3g22810.1 of Arabidopsis thaliana.

SEQ ID NO:36 is an amino acid sequence encoded by the locus At3g63300.1 of Arabidopsis thaliana.

SEQ ID NO:37 is the amino acid sequence corresponding to the locus LOC_Os03g43510.1, a rice (japonica) predicted protein from the Michigan State University Rice Genome Annotation Project Osa1 release 6.

SEQ ID NO:38 is the amino acid sequence corresponding to the locus LOC_Os12g41140.1, a rice (japonica) predicted protein from the Michigan State University Rice Genome Annotation Project Osa1 release 6.

SEQ ID NO:39 is the amino acid sequence corresponding to the locus LOC_Os01g13070.1, a rice (japonica) predicted protein from the Michigan State University Rice Genome Annotation Project Osa1 release 6.

SEQ ID NO:40 is the amino acid sequence corresponding to Sb01g014000.1, a sorghum (Sorghum bicolor) predicted protein from the Sorghum JGI genomic sequence version 1.4 from the US Department of Energy Joint Genome Institute.

SEQ ID NO:41 is the amino acid sequence corresponding to Sb03g000810.1, a sorghum (Sorghum bicolor) predicted protein from the Sorghum JGI genomic sequence version 1.4 from the US Department of Energy Joint Genome Institute.

SEQ ID NO:42 is the amino acid sequence corresponding to Sb01g029090.1, a sorghum (Sorghum bicolor) predicted protein from the Sorghum JGI genomic sequence version 1.4 from the US Department of Energy Joint Genome Institute.

SEQ ID NO:43 is the amino acid sequence corresponding to Sb04g032910.1, a sorghum (Sorghum bicolor) predicted protein from the Sorghum JGI genomic sequence version 1.4 from the US Department of Energy Joint Genome Institute.

SEQ ID NO:44 is the amino acid sequence corresponding to Glyma10g38990.1, a soybean (Glycine max) predicted protein from predicted coding sequences from Soybean JGI Glyma1.01 genomic sequence from the US Department of Energy Joint Genome Institute.

SEQ ID NO:45 is the amino acid sequence corresponding to Glyma02g00760.1, a soybean (Glycine max) predicted protein from predicted coding sequences from Soybean JGI Glyma1.01 genomic sequence from the US Department of Energy Joint Genome Institute.

SEQ ID NO:46 is the amino acid sequence corresponding to Glyma20g28820.1, a soybean (Glycine max) predicted protein from predicted coding sequences from Soybean JGI Glyma1.01 genomic sequence from the US Department of Energy Joint Genome Institute.

SEQ ID NO:47 is the amino acid sequence corresponding to Glyma10g00600.1, a soybean (Glycine max) predicted protein from predicted coding sequences from Soybean JGI Glyma1.01 genomic sequence from the US Department of Energy Joint Genome Institute.

SEQ ID NO:48 is the amino acid sequence corresponding to Glyma03g31340.1, a soybean (Glycine max) predicted protein from predicted coding sequences from Soybean JGI Glyma1.01 genomic sequence from the US Department of Energy Joint Genome Institute.

SEQ ID NO:49 is the amino acid sequence corresponding to Glyma19g34180.1, a soybean (Glycine max) predicted protein from predicted coding sequences from Soybean JGI Glyma1.01 genomic sequence from the US Department of Energy Joint Genome Institute.

SEQ ID NO:50 is the amino acid sequence corresponding to Glyma02g16330.1, a soybean (Glycine max) predicted protein from predicted coding sequences from Soybean JGI Glyma1.01 genomic sequence from the US Department of Energy Joint Genome Institute.

SEQ ID NO:51 is the amino acid sequence corresponding to Glyma10g03500.1, a soybean (Glycine max) predicted protein from predicted coding sequences from Soybean JGI Glyma1.01 genomic sequence from the US Department of Energy Joint Genome Institute.

SEQ ID NO:52 is the amino acid sequence corresponding to SEQ ID NO:1647 from the US patent publication No. US20120227134.

SEQ ID NO:53 is the amino acid sequence corresponding to SEQ ID NO:1566 from the US patent publication No. US20120096584.

SEQ ID NO:54 is the amino acid sequence corresponding to NCBI GI No. 195656831.

SEQ ID NO:55 is the amino acid sequence corresponding to SEQ ID NO:68801 from the US patent publication No. US20120017338.

SEQ ID NO:56 is the amino acid sequence corresponding to NCBI GI No. 293336271.

SEQ ID NO:57 is the amino acid sequence corresponding to SEQ ID NO:58064 from the US patent publication No. US20120017292.

SEQ ID NO:58 is the amino acid sequence corresponding to NCBI GI No. 514754743.

SEQ ID NO:59 is the amino acid sequence corresponding to SEQ ID NO:43844 from the US patent publication No. US20120017338.

SEQ ID NO:60 is the amino acid sequence corresponding to NCBI GI No. 242055855.

SEQ ID NO:61 is the amino acid sequence corresponding to SEQ ID NO:174 from the PCT publication No. WO2013072832.

SEQ ID NO:62 is the amino acid sequence corresponding to Uniprot Accession No. K7UXW2.

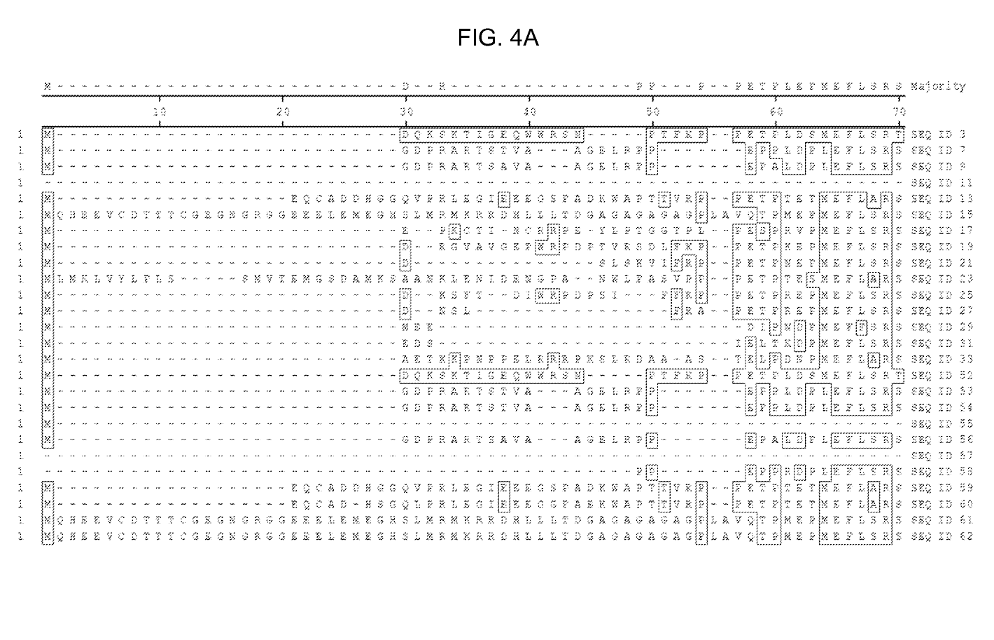

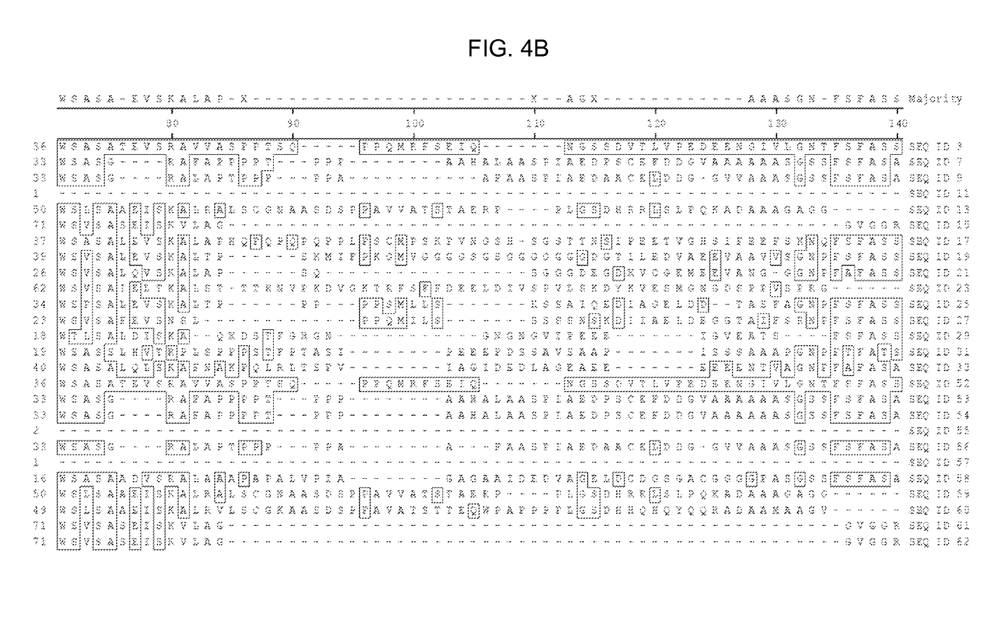

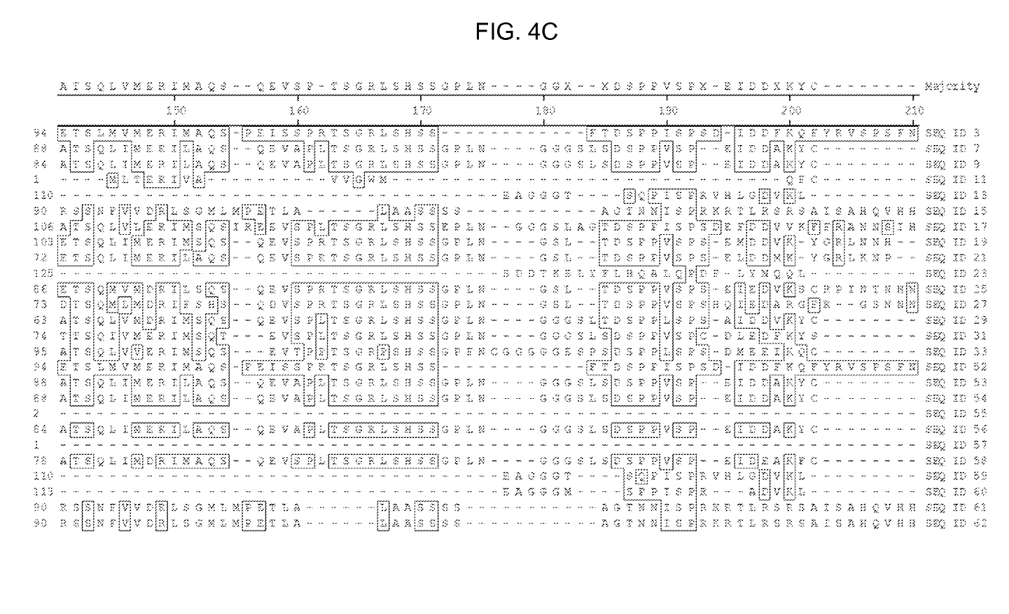

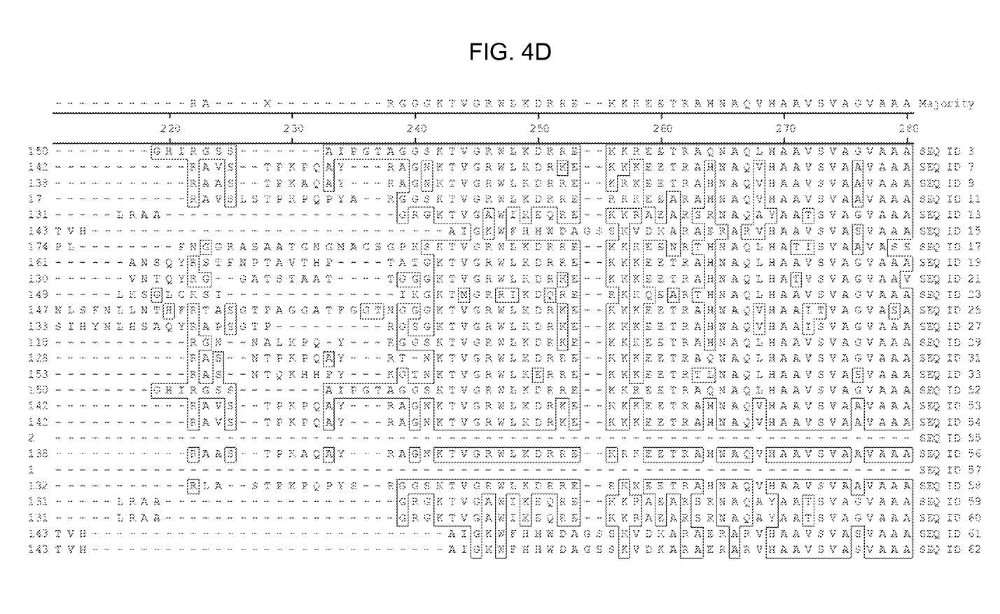

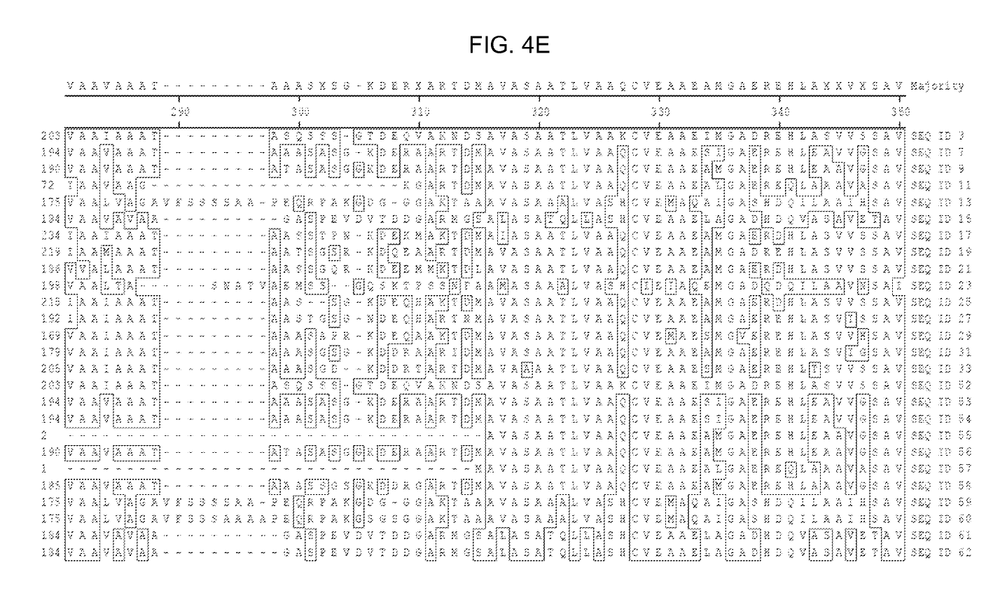

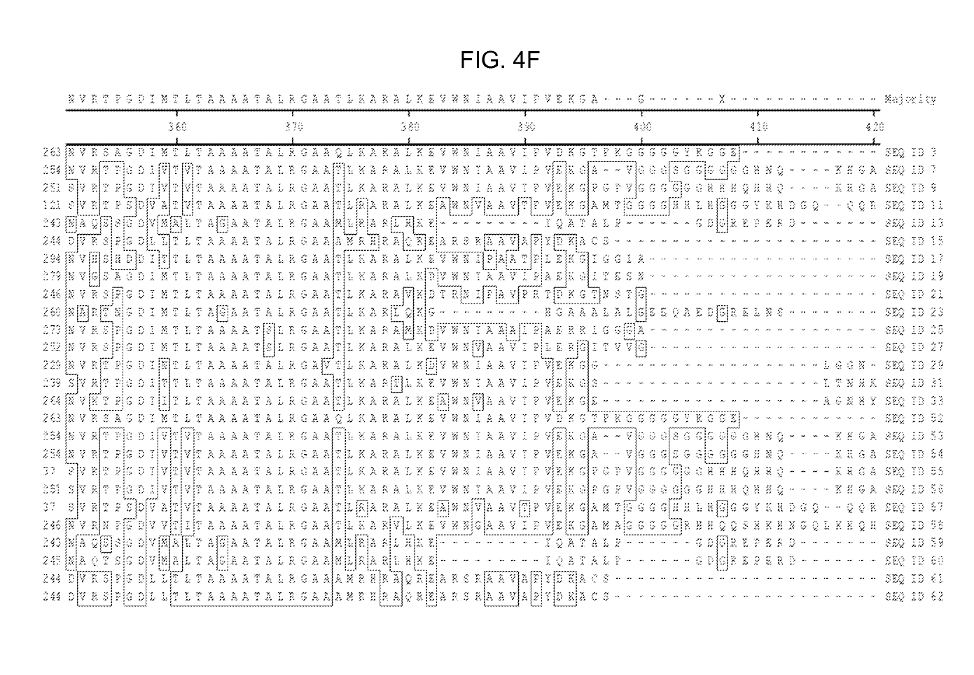

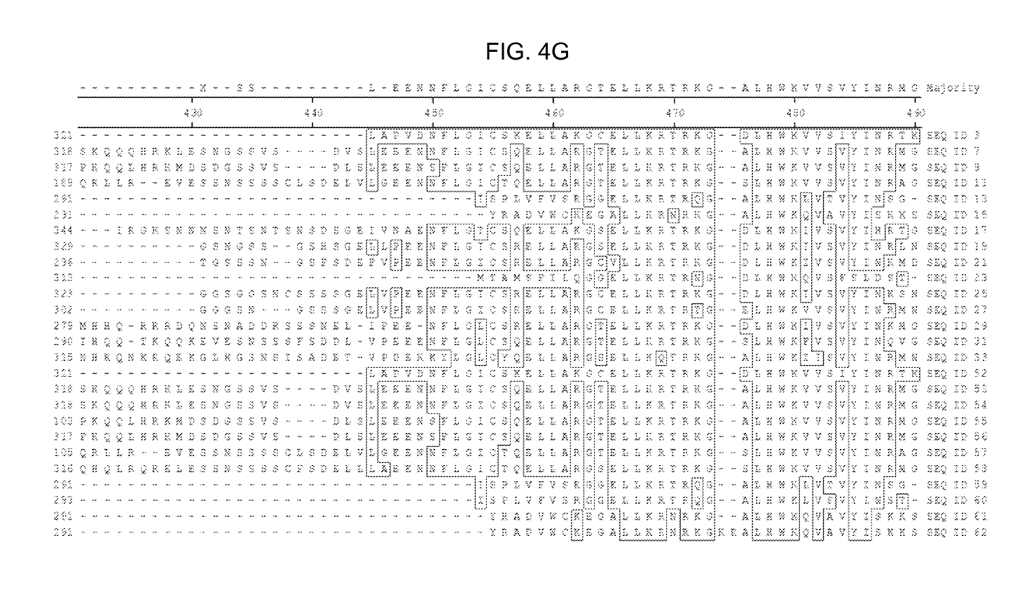

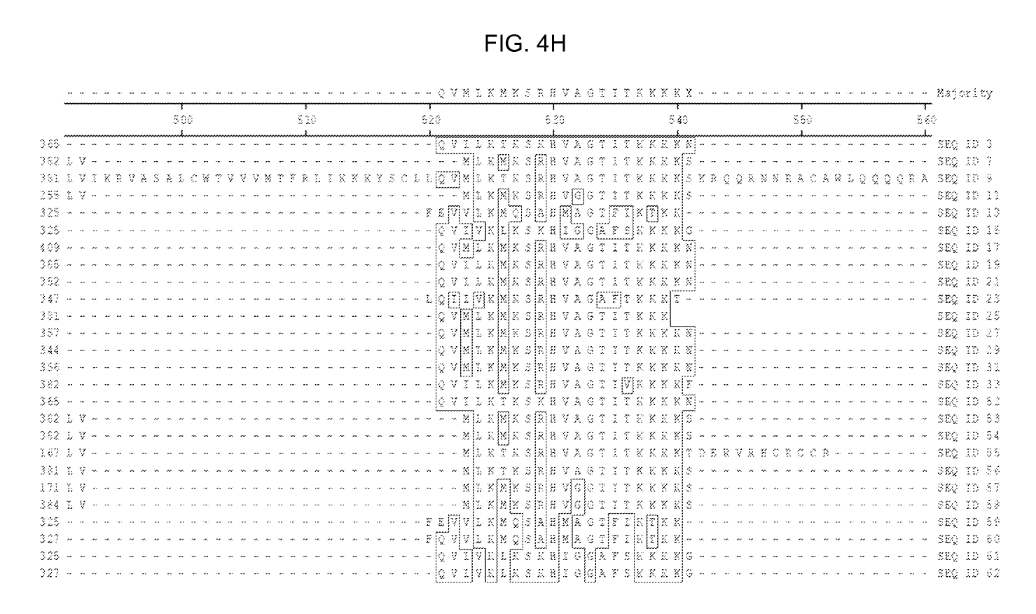

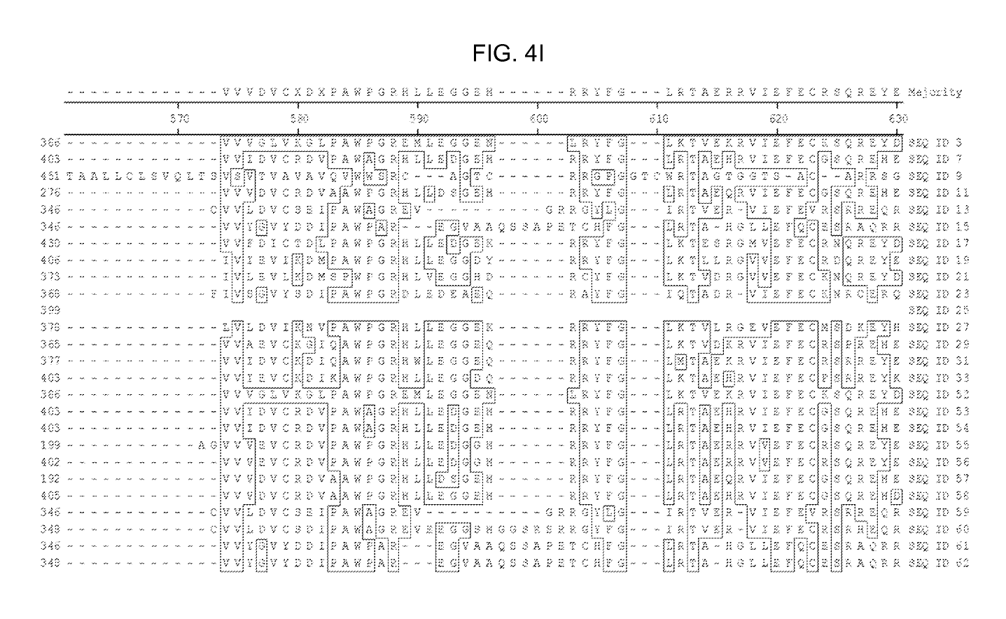

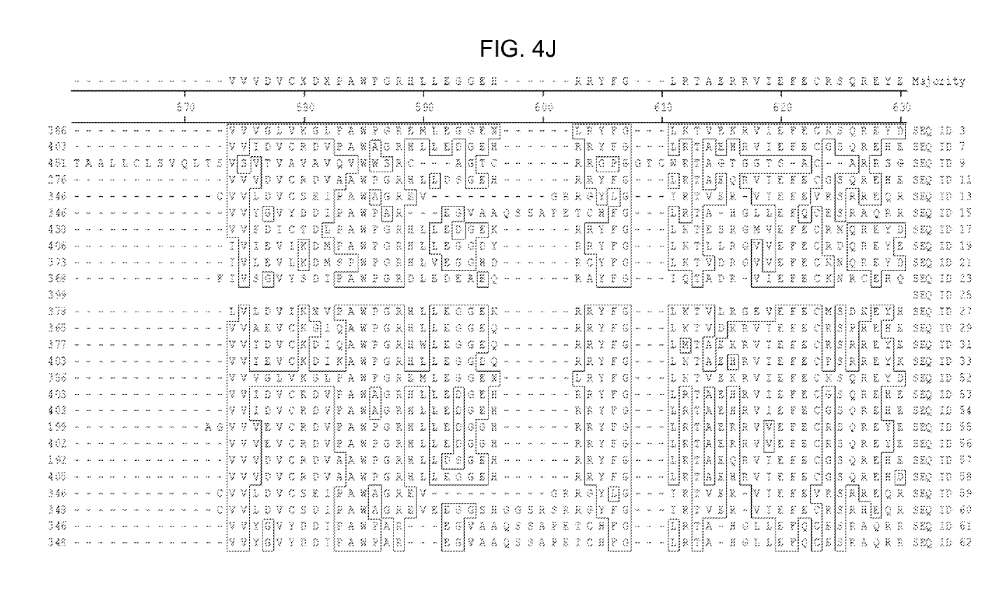

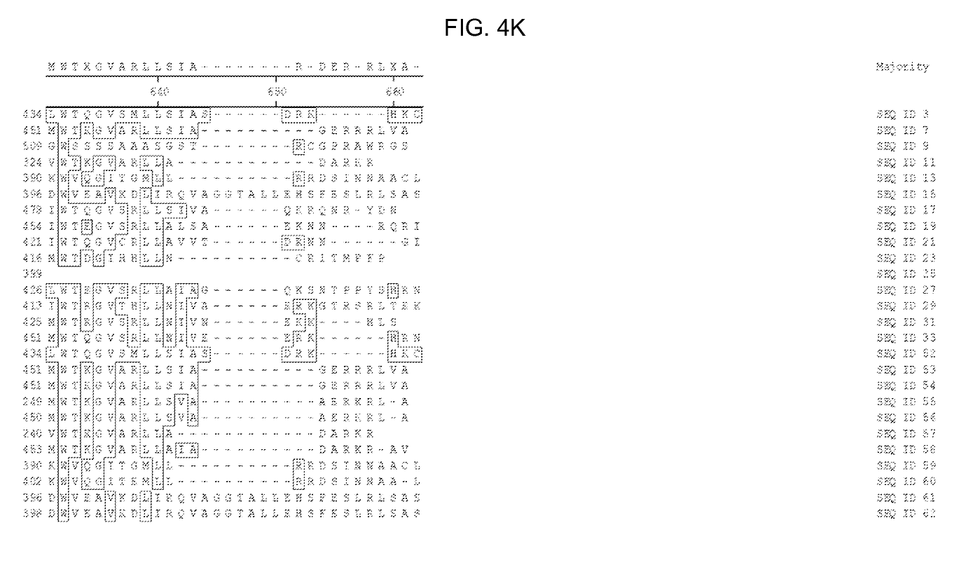

SEQ ID NO:63 is the consensus sequence obtained by aligning the SEQ ID NOS:3, 7, 9, 11, 13, 15, 17, 19, 21, 23, 25, 27, 29, 31, 33, 52-61 and 62, as shown in FIGS. 4A-4K.

SEQ ID NO:64 is the sequence of the Arabidopsis Phosphate Transporter 1 (PHT1) promoter.

SEQ ID NO:65 is the sequence of the Arabidopsis Oxygen evolving enhancer protein 2 (OEE2) promoter.

SEQ ID NO:66 is the sequence of the Arabidopsis Sucrose Transporter (SUC2) promoter.

SEQ ID NO:67 is the nucleotide sequence of the At2g36030-5′ attB forward primer.

SEQ ID NO:68 is the nucleotide sequence of the At2g36030-3′ attB reverse primer.

SEQ ID NO:69 is the nucleotide sequence of AT-NUCPU29 cDNA (identical to the CDS sequence from the Arabidopsis thaliana locus At2g36030), and corresponds to NCBI GI no. 18404023.

SEQ ID NO:70 is the amino acid sequence of the polypeptide encoded by SEQ ID NO:69, and corresponds to NCBI GI no. 15227254.

SEQ ID NO:71 is the AT-NUCPU29 nucleotide sequence with alternative codons (AT-NUCPU29ac). In SEQ ID NO:71 the nucleotide sequence encoding the NUCPU29 polypeptide has been altered to utilize maize-preferred codons.

SEQ ID NO:72 is the nucleotide sequence of the attB1 site.

SEQ ID NO:73 is the nucleotide sequence of the attB2 site.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

The terms “monocot” and “monocotyledonous plant” are used interchangeably herein. A monocot of the current disclosure includes the Gramineae.

The terms “dicot” and “dicotyledonous plant” are used interchangeably herein. A dicot of the current disclosure includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

A “trait” refers to a physiological, morphological, biochemical, or physical characteristic of a plant or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Nitrogen limiting conditions” refers to conditions where the amount of total available nitrogen (e.g., from nitrates, ammonia, or other known sources of nitrogen) is not sufficient to sustain optimal plant growth and development. One skilled in the art would recognize conditions where total available nitrogen is sufficient to sustain optimal plant growth and development. One skilled in the art would recognize what constitutes sufficient amounts of total available nitrogen, and what constitutes soils, media and fertilizer inputs for providing nitrogen to plants. Nitrogen limiting conditions will vary depending upon a number of factors, including but not limited to, the particular plant and environmental conditions.

“Agronomic characteristic” is a measurable parameter including but not limited to, abiotic stress tolerance, nitrogen stress tolerance, greenness, yield, growth rate, biomass, fresh weight at maturation, dry weight at maturation, fruit yield, seed yield, total plant nitrogen content, fruit nitrogen content, seed nitrogen content, nitrogen content in vegetative tissue, whole plant amino acid content, vegetative tissue free amino acid content, fruit free amino acid content, total plant protein content, seed free amino acid content, fruit protein content, seed protein content, protein content in a vegetative tissue, drought tolerance, nitrogen uptake, resistance to root lodging, root biomass, average root length, harvest index, stalk lodging, plant height, ear height, ear length, salt tolerance, early seedling vigor, and seedling emergence under low temperature stress.

Abiotic stress may be at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., paraquat) that induce production of reactive oxygen species (ROS). Nutrients include, but are not limited to, the following: nitrogen (N), phosphorus (P), potassium (K), calcium (Ca), magnesium (Mg) and sulfur (S).

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions.

“Stress tolerance activity” of a polypeptide indicates that over-expression of the polypeptide in a transgenic plant confers increased stress tolerance to the transgenic plant relative to a reference or control plant.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

Increased leaf size may be of particular interest. Increasing leaf biomass can be used to increase production of plant-derived pharmaceutical or industrial products. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach water or nutrients or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits in the forms of greater yield or improved screening.

“Harvest index” refers to the grain weight divided by the total plant weight.

The term “AT-PH11 polynucleotide” as used herein refers to the CDS (NCBI GI NO. 186529090; SEQ ID NO:2) encoding AT-PH11 polypeptide (SEQ ID NO:3; NCBI GI NO. 15240022), from the Arabidopsis thaliana gene locus At5g43870.

The term “PH11 polynucleotide” as used herein refers to the CDS (NCBI GI NO. 186529090; SEQ ID NO:2) encoding the AT-PH11 polypeptide (SEQ ID NO:3; NCBI GI NO. 15240022), and to polynucleotides that encode homologs of the Arabidopsis thaliana PH11 polypeptide, from the same and/or different species.

The term “AT-PH11 polypeptide” as used herein refers to the protein (SEQ ID NO:3; NCBI GI NO. 15240022) encoded by the Arabidopsis thaliana locus At5g43870 (SEQ ID NO:2 (AT-PH11 CDS sequence), SEQ ID NO:4 (AT-PH11 with alternate codons) or SEQ ID NO:5 (AT-PH11 cDNA sequence).

Genes encoding PH11 polypeptides include without limitation the Arabidopsis thaliana gene At5g43870 (SEQ ID NO:2), and its nucleotide homologs.

The term “PH11 polypeptides”, refers to homologs of AT-PH11, from the same or different species.

AT-PH11 contains a Pleckstrin-homology domain, and is a putative Phosphoinositide Binding protein (PBP) (Leeuwen et al. (2004) Trends in Plant Science Vol.9 No. 8 Aug. 2004). Benschop et al. (Mol. Cell. Proteom. (2007) 6:1198-1214) have shown that peptides from the At5g43870 polypeptide are phosphorylated upon xylanase or flagellin peptide treatment of Arabidopsis cells. One of the AT-PH11 homologs from Arabidopsis, At3g63300, encodes the FORKED1 protein that has been shown to be involved in auxin transport (Hou et al., Plant Journal (2010) 63:960-973).

The term “AT-NUCPU29 polynucleotide” as used herein refers to the cDNA (NCBI GI NO. 18404023; SEQ ID NO:69) encoding the AT-NUCPU29 polypeptide (SEQ ID NO:70; NCBI GI NO. 15227560), from the Arabidopsis thaliana gene locus At2g36030.

The term “NUCPU29 polynucleotide” as used herein refers to the cDNA (NCBI GI NO. 18404023; SEQ ID NO:69) encoding AT-NUCPU29 polypeptide (SEQ ID NO:70; NCBI GI NO. 15227560), and to polynucleotides that encode homologs of the Arabidopsis thaliana NUCPU29 polypeptide, from the same and/or different species.

The term “AT-NUCPU29 polypeptide” as used herein refers to the protein (SEQ ID NO:70; NCBI GI NO. 15227560) encoded by the Arabidopsis thaliana locus At2g36030 (SEQ ID NO:69).

Genes encoding NUCPU29 polypeptides include without limitation the Arabidopsis thaliana gene At2g36030 (SEQ ID NO:69), and its nucleotide homologs.

The term “NUCPU29 polypeptides”, refers to the AT-NUCPU29 polypeptide and its homologs, from the same or different species.

Katz et al. (Mol. Cell. Proteom. 2.8 (2003) 525-540; supplementary data) have predicted the possibility of the polypeptide encoded by At2g36030 to be a methyltransferase, by computational analysis.

“Splice variants” used herein refers to alternative forms of RNA transcribed from a gene. Splice variation naturally occurs as a result of alternative sites being spliced within a single transcribed RNA molecule or between separately transcribed RNA molecules, and may result in several different forms of mRNA transcribed from the same gene. Thus, splice variants may encode polypeptides having different amino acid sequences, which may or may not have similar functions in the organism.

“Nitrogen stress tolerance” is a trait of a plant and refers to the ability of the plant to survive under nitrogen limiting conditions.

“Increased nitrogen stress tolerance” of a plant is measured relative to a reference or control plant, and means that the nitrogen stress tolerance of the plant is increased by any amount or measure when compared to the nitrogen stress tolerance of the reference or control plant.

A “nitrogen stress tolerant plant” is a plant that exhibits nitrogen stress tolerance. A nitrogen stress tolerant plant may be a plant that exhibits an increase in at least one agronomic characteristic relative to a control plant under nitrogen limiting conditions.

“Environmental conditions” refer to conditions under which the plant is grown, such as the availability of water, availability of nutrients (for example nitrogen), or the presence of insects or disease.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. Gene stacking can be accomplished by many means including but not limited to co-transformation, retransformation, and crossing lines with different transgenes.

“Transgenic plant” also includes reference to plants which comprise more than one heterologous polynucleotide within their genome. Each heterologous polynucleotide may confer a different trait to the transgenic plant.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably to refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“CDS” refers to the coding sequence of a gene.

“Messenger RNA (mRNA)” refers to the RNA that is without introns and that can be translated into protein by the cell.

“cDNA” refers to a DNA that is complementary to and synthesized from an mRNA template using the enzyme reverse transcriptase. The cDNA can be single-stranded or converted into the double-stranded form using the Klenow fragment of DNA polymerase I.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

“Coding region” refers to the portion of a messenger RNA (or the corresponding portion of another nucleic acid molecule such as a DNA molecule) which encodes a protein or polypeptide. “Non-coding region” refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein refers to a post-translationally processed polypeptide; i.e., one from which any pre- or pro-peptides present in the primary translation product have been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

The terms “entry clone” and “entry vector” are used interchangeably herein.

“Regulatory sequences” or “regulatory elements” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Developmentally regulated promoter” refers to a promoter whose activity is determined by developmental events.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

A “chloroplast transit peptide” is an amino acid sequence which is translated in conjunction with a protein and directs the protein to the chloroplast or other plastid types present in the cell in which the protein is made (Lee et al. (2008) Plant Cell 20:1603-1622). The terms “chloroplast transit peptide” and “plastid transit peptide” are used interchangeably herein. “Chloroplast transit sequence” refers to a nucleotide sequence that encodes a chloroplast transit peptide. A “signal peptide” is an amino acid sequence which is translated in conjunction with a protein and directs the protein to the secretory system (Chrispeels (1991) Ann. Rev. Plant Phys. Plant Mol. Biol. 42:21-53). If the protein is to be directed to a vacuole, a vacuolar targeting signal (supra) can further be added, or if to the endoplasmic reticulum, an endoplasmic reticulum retention signal (supra) may be added. If the protein is to be directed to the nucleus, any signal peptide present should be removed and instead a nuclear localization signal included (Raikhel (1992) Plant Phys. 100:1627-1632). A “mitochondrial signal peptide” is an amino acid sequence which directs a precursor protein into the mitochondria (Zhang and Glaser (2002) Trends Plant Sci 7:14-21).

Sequence alignments and percent identity calculations may be determined using a variety of comparison methods designed to detect homologous sequences including, but not limited to, the MEGALIGN® program of the LASERGENE® bioinformatics computing suite (DNASTAR Inc., Madison, Wis.). Unless stated otherwise, multiple alignment of the sequences provided herein were performed using the Clustal V method of alignment (Higgins and Sharp, CABIOS. 5:151-153 (1989)) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MEGALIGN® v6.1 program of the LASERGENE® bioinformatics computing suite (DNASTAR Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Turning now to the embodiments:

Embodiments include isolated polynucleotides and polypeptides, recombinant DNA constructs, compositions (such as plants or seeds) comprising these recombinant DNA constructs, and methods utilizing these recombinant DNA constructs.

Isolated Polynucleotides and Polypeptides

The present disclosure includes the following isolated polynucleotides and polypeptides:

An isolated polynucleotide comprising: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; or (ii) a full complement of the nucleic acid sequence of (i), wherein the full complement and the nucleic acid sequence of (i) consist of the same number of nucleotides and are 100% complementary. Any of the foregoing isolated polynucleotides may be utilized in any recombinant DNA constructs (including suppression DNA constructs) of the present disclosure. The polypeptide is preferably a PH11 or NUCPU29 polypeptide. The PH11 or NUCPU29 polypeptide preferably has nitrogen stress tolerance activity.

An isolated polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70. The polypeptide is preferably a PH11 or NUCPU29 polypeptide. The PH11 or NUCPU29 polypeptide preferably has nitrogen stress tolerance activity.

An isolated polynucleotide comprising (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 24, 26, 28, 30, 32, 69 or 71; or (ii) a full complement of the nucleic acid sequence of (i). Any of the foregoing isolated polynucleotides may be utilized in any recombinant DNA constructs (including suppression DNA constructs) of the present disclosure. The isolated polynucleotide preferably encodes a PH11 or NUCPU29 polypeptide. The PH11 or NUCPU29 polypeptide preferably has nitrogen stress tolerance activity.

Recombinant DNA Constructs and Suppression DNA Constructs

In one aspect, the present disclosure includes recombinant DNA constructs (including suppression DNA constructs).

In one embodiment, a recombinant DNA construct comprises a polynucleotide operably linked to at least one heterologous regulatory sequence (e.g., a promoter functional in a plant), wherein the polynucleotide comprises (i) a nucleic acid sequence encoding an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; or (ii) a full complement of the nucleic acid sequence of (i).

In another embodiment, a recombinant DNA construct comprises a polynucleotide operably linked to at least one heterologous regulatory sequence (e.g., a promoter functional in a plant), wherein said polynucleotide comprises (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 24, 26, 28, 30, 32, 69 or 71; or (ii) a full complement of the nucleic acid sequence of (i)

In another embodiment, a recombinant DNA construct comprises a polynucleotide operably linked to at least one heterologous regulatory sequence (e.g., a promoter functional in a plant), wherein said polynucleotide encodes a PH11 or NUCPU29 polypeptide. The PH11 or NUCPU29 polypeptide preferably has nitrogen stress tolerance activity. The PH11 or NUCPU29 polypeptide may be from Arabidopsis thaliana, Zea mays, Glycine max, Glycine tabacina, Glycine soja, Glycine tomentella, Oryza sativa, Brassica napus, Sorghum bicolor, Saccharum officinarum, or Triticum aestivum.

In one embodiment, the polynucleotides described in the current disclosure are operably linked to a tissue-specific promoter. In one embodiment, the polynucleotides described in the current disclosure are operably linked to a promoter that is preferably expressed in any one or more of these tissues: root, shoot and vasculature.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The protein of the present disclosure may also be a protein which is encoded by a nucleic acid comprising a nucleotide sequence hybridizable under stringent conditions with the complementary strand of any of the nucleotide sequences of the present disclosure.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

In another aspect, the present disclosure includes suppression DNA constructs.

A suppression DNA construct may comprise at least one heterologous regulatory sequence (for example, a promoter functional in a plant) operably linked to (a) all or part of: (i) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; or (ii) a full complement of the nucleic acid sequence of (a)(i); or (b) a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a PH11 or NUCPU29 protein; or (c) all or part of: (i) a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:2, 4, 6, 8, 10, 12, 14, 16, 18, 20, 22, 24, 26, 28, 30, 32, 69 or 71; or (ii) a full complement of the nucleic acid sequence of (c)(i). The suppression DNA construct may comprise a cosuppression construct, antisense construct, viral-suppression construct, hairpin suppression construct, stem-loop suppression construct, double-stranded RNA-producing construct, RNAi construct, or small RNA construct (e.g., an sRNA construct or an miRNA construct).

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, includes lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

A suppression DNA construct may comprise a region derived from a target gene of interest and may comprise all or part of the nucleic acid sequence of the sense strand (or antisense strand) of the target gene of interest. Depending upon the approach to be utilized, the region may be 100% identical or less than 100% identical (e.g., at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical) to all or part of the sense strand (or antisense strand) of the gene of interest.

A suppression DNA construct may comprise 100, 200, 300, 400, 500, 600, 700, 800, 900 or 1000 contiguous nucleotides of the sense strand (or antisense strand) of the gene of interest, and combinations thereof.

Suppression DNA constructs are well-known in the art, are readily constructed once the target gene of interest is selected, and include, without limitation, cosuppression constructs, antisense constructs, viral-suppression constructs, hairpin suppression constructs, stem-loop suppression constructs, double-stranded RNA-producing constructs, and more generally, RNAi (RNA interference) constructs and small RNA constructs such as sRNA (short interfering RNA) constructs and miRNA (microRNA) constructs.

Suppression of gene expression may also be achieved by use of artificial miRNA precursors, ribozyme constructs and gene disruption. A modified plant miRNA precursor may be used, wherein the precursor has been modified to replace the miRNA encoding region with a sequence designed to produce a miRNA directed to the nucleotide sequence of interest. Gene disruption may be achieved by use of transposable elements or by use of chemical agents that cause site-specific mutations.

“Antisense inhibition” refers to the production of antisense RNA transcripts capable of suppressing the expression of the target gene or gene product. “Antisense RNA” refers to an RNA transcript that is complementary to all or part of a target primary transcript or mRNA and that blocks the expression of a target isolated nucleic acid fragment (U.S. Pat. No. 5,107,065). The complementarity of an antisense RNA may be with any part of the specific gene transcript, i.e., at the 5′ non-coding sequence, 3′ non-coding sequence, introns, or the coding sequence.

“Cosuppression” refers to the production of sense RNA transcripts capable of suppressing the expression of the target gene or gene product. “Sense” RNA refers to RNA transcript that includes the mRNA and can be translated into protein within a cell or in vitro. Cosuppression constructs in plants have been previously designed by focusing on overexpression of a nucleic acid sequence having homology to a native mRNA, in the sense orientation, which results in the reduction of all RNA having homology to the overexpressed sequence (see Vaucheret et al., Plant J. 16:651-659 (1998); and Gura, Nature 404:804-808 (2000)).

Another variation describes the use of plant viral sequences to direct the suppression of proximal mRNA encoding sequences (PCT Publication No. WO 98/36083 published on Aug. 20, 1998).

RNA interference refers to the process of sequence-specific post-transcriptional gene silencing in animals mediated by short interfering RNAs (siRNAs) (Fire et al., Nature 391:806 (1998)). The corresponding process in plants is commonly referred to as post-transcriptional gene silencing (PTGS) or RNA silencing and is also referred to as quelling in fungi. The process of post-transcriptional gene silencing is thought to be an evolutionarily-conserved cellular defense mechanism used to prevent the expression of foreign genes and is commonly shared by diverse flora and phyla (Fire et al., Trends Genet. 15:358 (1999)).

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes. Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

The terms “miRNA-star sequence” and “miRNA*sequence” are used interchangeably herein and they refer to a sequence in the miRNA precursor that is highly complementary to the miRNA sequence. The miRNA and miRNA*sequences form part of the stem region of the miRNA precursor hairpin structure.

Regulatory Sequences:

A recombinant DNA construct (including a suppression DNA construct) of the present disclosure may comprise at least one heterologous regulatory sequence.

A regulatory sequence may be a promoter.

A number of promoters can be used in recombinant DNA constructs (and suppression DNA constructs) of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may (or may not) have pleiotropic effects, although candidate gene efficacy may be estimated when driven by a constitutive promoter. Use of tissue-specific and/or stress-specific promoters may eliminate undesirable effects, but retain the ability to enhance nitrogen tolerance. This type of effect has been observed in Arabidopsis for drought and cold tolerance (Kasuga et al., Nature Biotechnol. 17:287-91 (1999)).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present disclosure which causes the desired temporal and spatial expression.

Promoters which are seed or embryo-specific and may be useful in the disclosure include soybean Kunitz trypsin inhibitor (Kti3, Jofuku and Goldberg, Plant Cell 1:1079-1093 (1989)), patatin (potato tubers) (Rocha-Sosa, M., et al., EMBO J. 8:23-29 (1989)), convicilin, vicilin, and legumin (pea cotyledons) (Rerie, W. G., et al., Mol. Gen. Genet. 259:149-157 (1991); Newbigin, E. J., et al., Planta 180:461-470 (1990); Higgins, T. J. V., et al., Plant. Mol. Biol. 11:683-695 (1988)), zein (maize endosperm) (Schemthaner, J. P., et al., EMBO J. 7:1249-1255 (1988)), phaseolin (bean cotyledon) (Segupta-Gopalan, C., et al., Proc. Natl. Acad. Sci. U.S.A. 82:3320-3324 (1995)), phytohemagglutinin (bean cotyledon) (Voelker, T. et al., EMBO J. 6:3571-3577 (1987)), B-conglycinin and glycinin (soybean cotyledon) (Chen, Z-L, et al., EMBO J. 7:297-302 (1988)), glutelin (rice endosperm), hordein (barley endosperm) (Marris, C., et al., Plant Mol. Biol. 10:359-366 (1988)), glutenin and gliadin (wheat endosperm) (Colot, V., et al., EMBO J. 6:3559-3564 (1987)), and sporamin (sweet potato tuberous root) (Hattori, T., et al., Plant Mol. Biol. 14:595-604 (1990)). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J. 6:3559-3564 (1987)).

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use in the current disclosure include the following: 1) the stress-inducible RD29A promoter (Kasuga et al., Nature Biotechnol. 17:287-91 (1999)); 2) the barley promoter, B22E; expression of B22E is specific to the pedicel in developing maize kernels (“Primary Structure of a Novel Barley Gene Differentially Expressed in Immature Aleurone Layers”, Klemsdal et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt et al., Plant Cell 5(7):729-737 (1993); “Structural characterization, chromosomal localization and phylogenetic evaluation of two pairs of AGAMOUS-like MADS-box genes from maize”, Theissen et al., Gene 156(2):155-166 (1995); NCBI GenBank Accession No. X80206)). Zag2 transcripts can be detected five days prior to pollination to seven to eight days after pollination (“DAP”), and directs expression in the carpel of developing female inflorescences and Ciml which is specific to the nucleus of developing maize kernels. Ciml transcript is detected four to five days before pollination to six to eight DAP. Other useful promoters include any promoter which can be derived from a gene whose expression is maternally associated with developing female florets.

Additional promoters for regulating the expression of the nucleotide sequences of the present disclosure in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

Promoters for use in the current disclosure may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (GenBank Accession No. EF030817), and the constitutive promoter GOS2 from Zea mays. Other promoters include root preferred promoters, such as the maize NAS2 promoter, the maize Cyclo promoter (US Publication No. 2006/0156439, published Jul. 13, 2006), the maize ROOTMET2 promoter (WO 2005/063998, published Jul. 14, 2005), the CR1BIO promoter (WO 2006/055487, published May 26, 2006), the CRWAQ81 (WO 2005/035770, published Apr. 21, 2005) and the maize ZRP2.47 promoter (NCBI Accession No. U38790; NCBI GI No. 1063664).

Recombinant DNA constructs (and suppression DNA constructs) of the present disclosure may also include other regulatory sequences including, but not limited to, translation leader sequences, introns, and polyadenylation recognition sequences. In another embodiment of the present disclosure, a recombinant DNA construct of the present disclosure further comprises an enhancer or silencer.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, Mol. Cell Biol. 8:4395-4405 (1988); Callis et al., Genes Dev. 1:1183-1200 (1987)).

Any plant can be selected for the identification of regulatory sequences and PH11 or NUCPU29 polypeptide genes to be used in recombinant DNA constructs of the present disclosure. Examples of suitable plant targets for the isolation of genes and regulatory sequences would include but are not limited to alfalfa, apple, apricot, Arabidopsis, artichoke, arugula, asparagus, avocado, banana, barley, beans, beet, blackberry, blueberry, broccoli, brussels sprouts, cabbage, canola, cantaloupe, carrot, cassava, castorbean, cauliflower, celery, cherry, chicory, cilantro, citrus, clementines, clover, coconut, coffee, corn, cotton, cranberry, cucumber, Douglas fir, eggplant, endive, escarole, eucalyptus, fennel, figs, garlic, gourd, grape, grapefruit, honey dew, jicama, kiwifruit, lettuce, leeks, lemon, lime, Loblolly pine, linseed, maize, mango, melon, mushroom, nectarine, nut, oat, oil palm, oil seed rape, okra, olive, onion, orange, an ornamental plant, palm, papaya, parsley, parsnip, pea, peach, peanut, pear, pepper, persimmon, pine, pineapple, plantain, plum, pomegranate, poplar, potato, pumpkin, quince, radiata pine, radicchio, radish, rapeseed, raspberry, rice, rye, sorghum, Southern pine, soybean, spinach, squash, strawberry, sugarbeet, sugarcane, sunflower, sweet potato, sweetgum, tangerine, tea, tobacco, tomato, triticale, turf, turnip, a vine, watermelon, wheat, yams, and zucchini.

Compositions:

A composition of the present disclosure includes a transgenic microorganism, cell, plant, and seed comprising the recombinant DNA construct. The cell may be eukaryotic, e.g., a yeast, insect or plant cell, or prokaryotic, e.g., a bacterial cell.

A composition of the present disclosure is a plant comprising in its genome any of the recombinant DNA constructs (including any of the suppression DNA constructs) of the present disclosure (such as any of the constructs discussed above). Compositions also include any progeny of the plant, and any seed obtained from the plant or its progeny, wherein the progeny or seed comprises within its genome the recombinant DNA construct (or suppression DNA construct). Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced recombinant DNA construct (or suppression DNA construct). These seeds can be grown to produce plants that would exhibit an altered agronomic characteristic (e.g., an increased agronomic characteristic optionally under nitrogen limiting conditions), or used in a breeding program to produce hybrid seed, which can be grown to produce plants that would exhibit such an altered agronomic characteristic. The seeds may be maize seeds.

The plant may be a monocotyledonous or dicotyledonous plant, for example, a maize or soybean plant, such as a maize hybrid plant or a maize inbred plant. The plant may also be sunflower, sorghum, canola, wheat, alfalfa, cotton, rice, barley, millet, sugar cane, or switchgrass.

The recombinant DNA construct may be stably integrated into the genome of the plant.

Particular embodiments include but are not limited to the following:

1. A plant (for example, a maize or soybean plant) comprising in its genome a recombinant DNA construct comprising a polynucleotide operably linked to at least one heterologous regulatory sequence, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70, and wherein said plant exhibits increased nitrogen stress tolerance when compared to a control plant not comprising said recombinant DNA construct. The plant may further exhibit an alteration of at least one agronomic characteristic when compared to the control plant.

2. A plant (for example, a maize or soybean plant) comprising in its genome a recombinant DNA construct comprising a polynucleotide operably linked to at least one heterologous regulatory sequence, wherein said polynucleotide encodes a PH11 or NUCPU29 polypeptide, and wherein said plant exhibits increased nitrogen stress tolerance when compared to a control plant not comprising said recombinant DNA construct. The plant may further exhibit an alteration of at least one agronomic characteristic when compared to the control plant.

3. A plant (for example, a maize or soybean plant) comprising in its genome a recombinant DNA construct comprising a polynucleotide operably linked to at least one heterologous regulatory sequence, wherein said polynucleotide encodes a PH11 or NUCPU29 polypeptide, and wherein said plant exhibits increased nitrogen stress tolerance when compared to a control plant not comprising said recombinant DNA construct. The plant may further exhibit an alteration of at least one agronomic characteristic when compared to the control plant.

4. A plant (for example, a maize or soybean plant) comprising in its genome a recombinant DNA construct comprising a polynucleotide operably linked to at least one heterologous regulatory sequence, wherein said polynucleotide encodes a PH11 or NUCPU29 polypeptide, and wherein said plant exhibits an alteration of at least one agronomic characteristic under nitrogen limiting conditions when compared to a control plant not comprising said recombinant DNA construct.

5. A plant (for example, a maize or soybean plant) comprising in its genome a recombinant DNA construct comprising a polynucleotide operably linked to at least one heterologous regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70, and wherein said plant exhibits an alteration of at least one agronomic characteristic under nitrogen limiting conditions when compared to a control plant not comprising said recombinant DNA construct.

6. A plant (for example, a maize or soybean plant) comprising in its genome a suppression DNA construct comprising at least one heterologous regulatory element operably linked to a region derived from all or part of a sense strand or antisense strand of a target gene of interest, said region having a nucleic acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to said all or part of a sense strand or antisense strand from which said region is derived, and wherein said target gene of interest encodes a PH11 or NUCPU29 polypeptide, and wherein said plant exhibits an alteration of at least one agronomic characteristic under nitrogen limiting conditions when compared to a control plant not comprising said suppression DNA construct.

7. A plant (for example, a maize or soybean plant) comprising in its genome a suppression DNA construct comprising at least one heterologous regulatory element operably linked to all or part of: (a) a nucleic acid sequence encoding a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; or (b) a full complement of the nucleic acid sequence of (a), and wherein said plant exhibits an alteration of at least one agronomic characteristic under nitrogen limiting conditions when compared to a control plant not comprising said suppression DNA construct.

8. In another embodiment, a plant comprising in its genome a recombinant DNA construct comprising a polynucleotide operably linked to at least one heterologous regulatory element, wherein said polynucleotide encodes a polypeptide having an amino acid sequence of at least 50% sequence identity, based on the Clustal V method of alignment, when compared to SEQ ID NO:3, 7, 9, 11, 13, 15, 17, 19, 21, 23, 25, 27, 29, 31, 33-62 or 70, and wherein said plant exhibits altered root architecture when compared to a control plant not comprising said recombinant DNA construct.

9. A plant (for example, a maize, rice or soybean plant) comprising in its genome a polynucleotide (optionally an endogenous polynucleotide) operably linked to at least one heterologous regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, %, 92%, 93%, 94%, 81%, 82%, 83%, 84%, 85%, 86%, 87%, 88%, 89%, 90%, 91 95%, 96%, 97%, 98%, 99%, or 100% sequence identity, based on the Clustal V or Clustal W method of alignment, when compared to SEQ ID NO:3, 7, 9, 11, 13, 15, 17, 19, 21, 23, 25, 27, 29, 31, 33-62 or 70, and wherein said plant exhibits at least one trait selected from the group consisting of: increased nitrogen stress tolerance, increased yield, increased biomass, and altered root architecture, when compared to a control plant not comprising the recombinant regulatory element. The at least one heterologous regulatory element may comprise an enhancer sequence or a multimer of identical or different enhancer sequences. The at least one heterologous regulatory element may comprise one, two, three or four copies of the CaMV 35S enhancer.

10. Any progeny of the plants described herein, any seeds of the plants described herein, any seeds of progeny of the plants described herein, and cells from any of the above plants described herein and progeny thereof.

In any of the plants, seeds or cells described herein, the PH11 or NUCPU29 polypeptide may be from Arabidopsis thaliana, Zea mays, Glycine max, Glycine tabacina, Glycine soja or Glycine tomentella Oryza sativa, Brassica napus, Sorghum bicolor, Saccharum officinarum, or Triticum aestivum.

In any of the plants, seeds or cells described herein, the recombinant DNA construct (or suppression DNA construct) may comprise at least a promoter functional in a plant as a regulatory sequence.

In any of the plants, seeds or cells described herein, the alteration of at least one agronomic characteristic is either an increase or decrease.

In any of the plants, seeds or cells described herein, the at least one agronomic characteristic may be selected from the group consisting of abiotic stress tolerance, nitrogen stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, whole plant amino acid content, vegetative tissue free amino acid content, fruit free amino acid content, seed free amino acid content, total plant protein content, fruit protein content, seed protein content, protein content in a vegetative tissue, drought tolerance, nitrogen uptake, resistance to root lodging, root biomass, average root length, harvest index, stalk lodging, plant height, ear height, ear length, salt tolerance, early seedling vigor, and seedling emergence under low temperature stress. For example, the alteration of at least one agronomic characteristic may be an increase in yield, greenness, or biomass.

“Yield” can be measured in many ways, including, for example, test weight, seed weight, seed number per plant, seed number per unit area (i.e. seeds, or weight of seeds, per acre), bushels per acre, tonnes per acre, tons per acre, kilo per hectare.

In any of the plants, seeds or cells described herein, the plant may exhibit the alteration of at least one agronomic characteristic when compared, under nitrogen stress conditions, to a control plant not comprising said recombinant DNA construct (or suppression DNA construct).

One of ordinary skill in the art is familiar with protocols for simulating nitrogen conditions, whether limiting or non-limiting, and for evaluating plants that have been subjected to simulated or naturally-occurring nitrogen conditions, whether limiting or non-limiting. For example, one can simulate nitrogen conditions by giving plants less nitrogen than normally required or no nitrogen over a period of time, and one can evaluate such plants by looking for differences in agronomic characteristics, e.g., changes in physiological and/or physical condition, including (but not limited to) vigor, growth, size, or root length, or in particular, leaf color or leaf area size. Other techniques for evaluating such plants include measuring chlorophyll fluorescence, photosynthetic rates, root growth or gas exchange rates.

The Examples below describe some representative protocols and techniques for simulating nitrogen limiting conditions and/or evaluating plants under such conditions.

One can also evaluate nitrogen stress tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring low or high nitrogen conditions (e.g., by measuring for substantially equivalent yield under low or high nitrogen conditions compared to normal nitrogen conditions, or by measuring for less yield loss under low or high nitrogen conditions compared to a control or reference plant).

One of ordinary skill in the art would readily recognize a suitable control or reference plant to be utilized when assessing or measuring an agronomic characteristic or phenotype of a transgenic plant in any embodiment of the present disclosure in which a control plant is utilized (e.g., compositions or methods as described herein). For example, by way of non-limiting illustrations:

1. Progeny of a transformed plant which is hemizygous with respect to a recombinant DNA construct (or suppression DNA construct), such that the progeny are segregating into plants either comprising or not comprising the recombinant DNA construct (or suppression DNA construct): the progeny comprising the recombinant DNA construct (or suppression DNA construct) would be typically measured relative to the progeny not comprising the recombinant DNA construct (or suppression DNA construct) (i.e., the progeny not comprising the recombinant DNA construct (or the suppression DNA construct) is the control or reference plant).

2. Introgression of a recombinant DNA construct (or suppression DNA construct) into an inbred line, such as in maize, or into a variety, such as in soybean: the introgressed line would typically be measured relative to the parent inbred or variety line (i.e., the parent inbred or variety line is the control or reference plant).

3. Two hybrid lines, where the first hybrid line is produced from two parent inbred lines, and the second hybrid line is produced from the same two parent inbred lines except that one of the parent inbred lines contains a recombinant DNA construct (or suppression DNA construct): the second hybrid line would typically be measured relative to the first hybrid line (i.e., the first hybrid line is the control or reference plant).

4. A plant comprising a recombinant DNA construct (or suppression DNA construct): the plant may be assessed or measured relative to a control plant not comprising the recombinant DNA construct (or suppression DNA construct) but otherwise having a comparable genetic background to the plant (e.g., sharing at least 90%, 91%, 92%, 93%, 94%, 95%, 96%, 97%, 98%, 99%, or 100% sequence identity of nuclear genetic material compared to the plant comprising the recombinant DNA construct (or suppression DNA construct)). There are many laboratory-based techniques available for the analysis, comparison and characterization of plant genetic backgrounds; among these are Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLP®s), and Simple Sequence Repeats (SSRs) which are also referred to as Microsatellites.

Furthermore, one of ordinary skill in the art would readily recognize that a suitable control or reference plant to be utilized when assessing or measuring an agronomic characteristic or phenotype of a transgenic plant would not include a plant that had been previously selected, via mutagenesis or transformation, for the desired agronomic characteristic or phenotype.

Methods:

Methods include but are not limited to methods for increasing nitrogen stress tolerance in a plant, methods for evaluating (or selecting for) nitrogen stress tolerance in a plant, methods for altering an agronomic characteristic in a plant, methods for determining (or selecting for) an alteration of an agronomic characteristic in a plant, and methods for producing seed. The plant may be a monocotyledonous or dicotyledonous plant, for example, a maize or soybean plant. The plant may also be sunflower, sorghum, canola, wheat, alfalfa, cotton, rice, barley, millet, sugar cane, or switchgrass. The seed may be a maize or soybean seed, for example a maize hybrid seed or maize inbred seed.

Methods include but are not limited to the following:

A method for transforming a cell (or microorganism) comprising transforming a cell (or microorganism) with any of the isolated polynucleotides or recombinant DNA constructs of the present disclosure. The cell (or microorganism) transformed by this method is also included. In particular embodiments, the cell is eukaryotic cell, e.g., a yeast, insect or plant cell, or prokaryotic, e.g., a bacterial cell. The microorganism may be Agrobacterium, e.g. Agrobacterium tumefaciens or Agrobacterium rhizogenes.

A method for producing a transgenic plant comprising transforming a plant cell with any of the isolated polynucleotides or recombinant DNA constructs (including suppression DNA constructs) of the present disclosure and regenerating a transgenic plant from the transformed plant cell. The disclosure is also directed to the transgenic plant produced by this method, and transgenic seed obtained from this transgenic plant. The transgenic plant obtained by this method may be used in other methods of the present disclosure.

A method for isolating a polypeptide of the disclosure from a cell or culture medium of the cell, wherein the cell comprises a recombinant DNA construct comprising a polynucleotide of the disclosure operably linked to at least one heterologous regulatory sequence, and wherein the transformed host cell is grown under conditions that are suitable for expression of the recombinant DNA construct.

A method of altering the level of expression of a polypeptide of the disclosure in a host cell comprising: (a) transforming a host cell with a recombinant DNA construct of the present disclosure; and (b) growing the transformed host cell under conditions that are suitable for expression of the recombinant DNA construct wherein expression of the recombinant DNA construct results in production of altered levels of the polypeptide of the disclosure in the transformed host cell.

A method of increasing nitrogen stress tolerance in a plant, comprising: (a) introducing into a regenerable plant cell a recombinant DNA construct comprising a polynucleotide operably linked to at least one heterologous regulatory sequence (for example, a promoter functional in a plant), wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; and (b) regenerating a transgenic plant from the regenerable plant cell after step (a), wherein the transgenic plant comprises in its genome the recombinant DNA. The method may further comprise (c) obtaining a progeny plant derived from the transgenic plant, wherein said progeny plant comprises in its genome the recombinant DNA construct and exhibits increased nitrogen stress tolerance when compared to a control plant not comprising the recombinant DNA construct.

A method of increasing nitrogen stress tolerance, the method comprising: (a) introducing into a regenerable plant cell a recombinant DNA construct comprising a polynucleotide operably linked to at least one heterologous regulatory element, wherein said polynucleotide comprises a nucleotide sequence, wherein the nucleotide sequence is: (a) hybridizable under stringent conditions with a DNA molecule comprising the full complement of SEQ ID NO:2, 4, 6, 8, 10, 12, 14, 16, 18, 20, 22, 24, 26, 28, 30, 32, 69 or 71; or (b) derived from SEQ ID NO:2, 4, 6, 8, 10, 12, 14, 16, 18, 20, 22, 24, 26, 28, 30, 32, 69 or 71, by alteration of one or more nucleotides by at least one method selected from the group consisting of: deletion, substitution, addition and insertion; and (b) regenerating a transgenic plant from the regenerable plant cell after step (a), wherein the transgenic plant comprises in its genome the recombinant DNA construct and exhibits increased nitrogen stress tolerance when compared to a control plant not comprising the recombinant DNA construct. The method may further comprise (c) obtaining a progeny plant derived from the transgenic plant, wherein said progeny plant comprises in its genome the recombinant DNA construct and exhibits increased nitrogen stress tolerance, when compared to a control plant not comprising the recombinant DNA construct.

A method of increasing nitrogen stress tolerance in a plant, comprising (a) obtaining a transgenic plant, wherein the transgenic plant comprises in its genome a recombinant DNA construct comprising a polynucleotide operably linked to at least one heterologous regulatory sequence (for example, a promoter functional in a plant), wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; (b) obtaining a progeny plant derived from the transgenic plant, wherein the progeny plant comprises in its genome the recombinant DNA construct; and; (c) evaluating (or selecting for) the progeny plant that exhibits nitrogen stress tolerance compared to a control plant not comprising the recombinant DNA construct.

A method of selecting for increased nitrogen stress tolerance in a plant, comprising (a) obtaining a transgenic plant, wherein the transgenic plant comprises in its genome a recombinant DNA construct comprising a polynucleotide operably linked to at least one heterologous regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; (b) obtaining a progeny plant derived from said transgenic plant, wherein the progeny plant comprises in its genome the recombinant DNA construct; and (c) selecting for the progeny plant with increased nitrogen stress tolerance compared to a control plant not comprising the recombinant DNA construct.

In another embodiment, a method of selecting for increased nitrogen stress tolerance in a plant, comprising: (a) obtaining a transgenic plant, wherein the transgenic plant comprises in its genome a recombinant DNA construct comprising a polynucleotide operably linked to at least one heterologous regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; (b) growing the transgenic plant of part (a) under conditions wherein the polynucleotide is expressed; and (c) selecting for the transgenic plant of part (b) with increased nitrogen stress tolerance compared to a control plant not comprising the recombinant DNA construct.

A method of selecting for increased nitrogen stress tolerance in a plant, the method comprising: (a) obtaining a transgenic plant, wherein the transgenic plant comprises in its genome a recombinant DNA construct comprising a polynucleotide operably linked to at least one heterologous regulatory element, wherein said polynucleotide comprises a nucleotide sequence, wherein the nucleotide sequence is: (i) hybridizable under stringent conditions with a DNA molecule comprising the full complement of SEQ ID NO:2, 4, 6, 8, 10, 12, 14, 16, 18, 20, 22, 24, 26, 28, 30, 32, 69 or 71; or (ii) derived from SEQ ID NO:2, 4, 6, 8, 10, 12, 14, 16, 18, 20, 22, 24, 26, 28, 30, 32, 69 or 71 by alteration of one or more nucleotides by at least one method selected from the group consisting of: deletion, substitution, addition and insertion; (b) obtaining a progeny plant derived from said transgenic plant, wherein the progeny plant comprises in its genome the recombinant DNA construct; and (c) selecting for the progeny plant with increased nitrogen stress tolerance, when compared to a control plant not comprising the recombinant DNA construct.

The use of a recombinant DNA construct for producing a plant that exhibits at least one trait selected from the group consisting of: increased nitrogen stress tolerance, increased yield, increased biomass, and altered root architecture, when compared to a control plant not comprising said recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO:3, 7, 9, 11, 13, 15, 17, 19, 21, 23, 25, 27, 29, 31, 33-62 or 70. The polypeptide may be over-expressed in at least one tissue of the plant, or during at least one condition of environmental stress, or both. The plant may be selected from the group consisting of: maize, soybean, sunflower, sorghum, canola, wheat, alfalfa, cotton, rice, barley, millet, sugar cane and switchgrass.

A method of altering root architecture in a plant, comprising: (a) introducing into regenerable plant cell a recombinant DNA construct comprising a polynucleotide operably linked to at least one heterologous regulatory sequence, wherein the polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70 (b) regenerating a transgenic plant from the regenerable plant cell after step (a), wherein the transgenic plant comprises in its genome the recombinant DNA construct; and (c) obtaining a progeny plant derived from the transgenic plant of step (b), wherein said progeny plant comprises in its genome the recombinant DNA construct and exhibits altered root architecture when compared to a control plant not comprising the recombinant DNA construct.

In another embodiment, a method of selecting for altered root architecture in a plant, comprising: (a) obtaining a transgenic plant, wherein the transgenic plant comprises in its genome a recombinant DNA construct comprising a polynucleotide operably linked to at least one heterologous regulatory element, wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; (b) selecting for a progeny plant derived from the transgenic plant, wherein the progeny plant comprises in its genome the recombinant DNA construct; and (c) evaluating (or selecting for) the progeny plant that exhibits alteration of root architecture compared to a control plant not comprising the recombinant DNA construct.

A method of selecting for altered root architecture in a plant, the method comprising: (a) obtaining a transgenic plant, wherein the transgenic plant comprises in its genome a recombinant DNA construct comprising a polynucleotide operably linked to at least one heterologous regulatory element, wherein said polynucleotide comprises a nucleotide sequence, wherein the nucleotide sequence is: (i) hybridizable under stringent conditions with a DNA molecule comprising the full complement of SEQ ID NO:2, 4, 6, 8, 10, 12, 14, 16, 18, 20, 22, 24, 26, 28, 30, 32, 69 or 71; or (ii) derived from SEQ ID NO:2, 4, 6, 8, 10, 12, 14, 16, 18, 20, 22, 24, 26, 28, 30, 32, 69 or 71 by alteration of one or more nucleotides by at least one method selected from the group consisting of: deletion, substitution, addition and insertion; (b) obtaining a progeny plant derived from said transgenic plant, wherein the progeny plant comprises in its genome the recombinant DNA construct; and (c) selecting for the progeny plant with altered root architecture, when compared to a control plant not comprising the recombinant DNA construct.

A method of selecting for an alteration of an agronomic characteristic in a plant, comprising (a) obtaining a transgenic plant, wherein the transgenic plant comprises in its genome a recombinant DNA construct comprising a polynucleotide operably linked to at least one heterologous regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; (b) obtaining a progeny plant derived from the transgenic plant, wherein the progeny plant comprises in its genome the recombinant DNA construct; and (c) selecting a progeny plant of (b) that exhibits an alteration of at least one agronomic characteristic when compared, optionally under nitrogen limiting conditions, to a control plant not comprising the recombinant DNA construct. The polynucleotide preferably encodes a PH11 or NUCPU29 polypeptide. The PH11 or NUCPU29 polypeptide preferably has nitrogen stress tolerance activity.

A method of selecting for an alteration of an agronomic characteristic in a plant, comprising (a) obtaining a transgenic plant, wherein the transgenic plant comprises in its genome a recombinant DNA construct comprising a polynucleotide operably linked to at least one heterologous regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; (b) growing the transgenic plant of part (a) under conditions wherein the polynucleotide is expressed; and (c) selecting a transgenic plant of part (b) that exhibits an alteration of at least one agronomic characteristic when compared, optionally under nitrogen limiting conditions, to a control plant not comprising the recombinant DNA construct.

A method of selecting for an alteration of an agronomic characteristic in a plant, comprising (a) obtaining a transgenic plant, wherein the transgenic plant comprises in its genome a recombinant DNA construct comprising a polynucleotide operably linked to at least one heterologous regulatory element, wherein said polynucleotide comprises a nucleotide sequence, wherein the nucleotide sequence is: (i) hybridizable under stringent conditions with a DNA molecule comprising the full complement of SEQ ID NO:2, 4, 6, 8, 10, 12, 14, 16, 18, 20, 22, 24, 26, 28, 30, 32, 69 or 71; or (ii) derived from SEQ ID NO:2, 4, 6, 8, 10, 12, 14, 16, 18, 20, 22, 24, 26, 28, 30, 32, 69 or 71 by alteration of one or more nucleotides by at least one method selected from the group consisting of: deletion, substitution, addition and insertion; (b) obtaining a progeny plant derived from said transgenic plant, wherein the progeny plant comprises in its genome the recombinant DNA construct; and (c) selecting (or identifying) the progeny plant that exhibits an alteration in at least one agronomic characteristic when compared, optionally under nitrogen limiting conditions, to a control plant not comprising the recombinant DNA construct. The polynucleotide preferably encodes a PH11 or NUCPU29 polypeptide. The PH11 or NUCPU29 polypeptide preferably has nitrogen stress tolerance activity.

A method of selecting for an alteration of an agronomic characteristic in a plant, comprising (a) introducing into a regenerable plant cell a recombinant DNA construct comprising a polynucleotide operably linked to at least one heterologous regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; (b) regenerating a transgenic plant from the regenerable plant cell after step (a), wherein the transgenic plant comprises in its genome said recombinant DNA construct; and (c) selecting a transgenic plant of (b) that exhibits an alteration of at least one agronomic characteristic when compared, optionally under nitrogen limiting conditions, to a control plant not comprising the recombinant DNA construct. The method may further comprise (d) obtaining a progeny plant derived from the transgenic plant, wherein the progeny plant comprises in its genome the recombinant DNA construct; and (e) selecting a progeny plant of (d) that exhibits an alteration of at least one agronomic characteristic when compared, optionally under nitrogen limiting conditions, to a control plant not comprising the recombinant DNA construct.

A method of selecting for an alteration of an agronomic characteristic in a plant, comprising (a) introducing into a regenerable plant cell a recombinant DNA construct comprising a polynucleotide operably linked to at least one heterologous regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V method of alignment, when compared to SEQ ID NO:3, 7, 9, 11, 13, 15, 17, 19, 21, 23, 25, 27, 29, 31, 33-62 or 70; (b) regenerating a transgenic plant from the regenerable plant cell after step (a), wherein the transgenic plant comprises in its genome said recombinant DNA construct; (c) obtaining a progeny plant derived from said transgenic plant, wherein the progeny plant comprises in its genome the recombinant DNA construct; and (d) selecting a progeny plant of (c) that exhibits an alteration of at least one agronomic characteristic when compared, optionally under nitrogen limiting conditions, to a control plant not comprising the recombinant DNA construct.

A method of producing a plant that exhibits an increase in at least one trait selected from the group consisting of: increased nitrogen stress tolerance, increased yield, increased biomass, and altered root architecture, wherein the method comprises growing a plant from a seed comprising a recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO:3, 7, 9, 11, 13, 15, 17, 19, 21, 23, 25, 27, 29, 31, 33-62 or 70, wherein the plant exhibits at least one trait selected from the group consisting of: increased nitrogen stress tolerance, increased yield, increased biomass, and altered root architecture, when compared to a control plant not comprising the recombinant DNA construct. The polypeptide may be over-expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

A method of producing a seed, the method comprising: (a) crossing a first plant with a second plant, wherein at least one of the first plant and the second plant comprises a recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO:3, 7, 9, 11, 13, 15, 17, 19, 21, 23, 25, 27, 29, 31, 33-62 or 70; and (b) selecting a seed of the crossing of step (a), wherein the seed comprises the recombinant DNA construct. A plant grown from the seed may exhibit at least one trait selected from the group consisting of: increased nitrogen stress tolerance, increased yield, increased biomass, and altered root architecture, when compared to a control plant not comprising the recombinant DNA construct. The polypeptide may be over-expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

A method of producing seed (for example, seed that can be sold as a nitrogen stress tolerant product offering) comprising any of the preceding methods, and further comprising obtaining seeds from said progeny plant, wherein said seeds comprise in their genome said recombinant DNA construct (or suppression DNA construct).

A method of producing oil or a seed by-product, or both, from a seed, the method comprising extracting oil or a seed by-product, or both, from a seed that comprises a recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO:3, 7, 9, 11, 13, 15, 17, 19, 21, 23, 25, 27, 29, 31, 33-62 or 70. The seed may be obtained from a plant that comprises the recombinant DNA construct, wherein the plant exhibits at least one trait selected from the group consisting of: increased nitrogen stress tolerance, increased yield, increased biomass, and altered root architecture, when compared to a control plant not comprising the recombinant DNA construct. The polypeptide may be over-expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass. The oil or the seed by-product, or both, may comprise the recombinant DNA construct.

Methods of isolating seed oils are well known in the art: (Young et al., Processing of Fats and Oils, In The Lipid Handbook, Gunstone et al., eds., Chapter 5 pp 253 257; Chapman & Hall: London (1994)). Seed by-products include but are not limited to the following: meal, lecithin, gums, free fatty acids, pigments, soap, stearine, tocopherols, sterols and volatiles.

One may evaluate altered root architecture in a controlled environment (e.g., greenhouse) or in field testing. The evaluation may be under simulated or naturally-occurring low or high nitrogen conditions. The altered root architecture may be an increase in root mass. The increase in root mass may be at least 5%, 6%, 7%, 8%, 9%, 10%, 11%, 12%, 13%, 14%, 15%, 16%, 17%, 18%, 19%, 20%, 21%, 22%, 23%, 24%, 25%, 30%, 35%, 40%, 45% or 50%, when compared to a control plant not comprising the recombinant DNA construct.

Also provided is the use of a recombinant DNA construct for producing a plant that exhibits at least one trait selected from the group consisting of: increased nitrogen stress tolerance, increased yield, increased biomass, and altered root architecture, when compared to a control plant not comprising said recombinant DNA construct, wherein the recombinant DNA construct comprises a polynucleotide operably linked to at least one heterologous regulatory element, wherein the polynucleotide encodes a polypeptide having an amino acid sequence of at least 70%, 75%, 80%, 85%, 90%, 91%, 92%, 93%, 94%, 95%, 96%, 97%, 98%, 99% or 100% sequence identity, based on the Clustal V or the Clustal W method of alignment, using the respective default parameters, when compared to SEQ ID NO:3, 7, 9, 11, 13, 15, 17, 19, 21, 23, 25, 27, 29, 31, 33-62 or 70. The polypeptide may be over-expressed in at least one tissue of the plant, or during at least one condition of abiotic stress, or both. The plant may be selected from the group consisting of: maize, soybean, sunflower, sorghum, canola, wheat, alfalfa, cotton, rice, barley, millet, sugar cane and switchgrass.

In any of the foregoing methods or any other embodiments of methods of the present disclosure, the step of selecting for an alteration of an agronomic characteristic in a transgenic plant (or progeny plant), if applicable, may comprise selecting a transgenic plant (or progeny plant) that exhibits an alteration of at least one agronomic characteristic when compared, under varying environmental conditions, to a control plant not comprising the recombinant DNA construct.

In any of the preceding methods or any other embodiments of methods of the present disclosure, in said introducing step said regenerable plant cell may comprise a callus cell, an embryogenic callus cell, a gametic cell, a meristematic cell, or a cell of an immature embryo. The regenerable plant cells may derive from an inbred maize plant.

In any of the preceding methods or any other embodiments of methods of the present disclosure, said regenerating step may comprise: (i) culturing said transformed plant cells in a media comprising an embryogenic promoting hormone until callus organization is observed; (ii) transferring said transformed plant cells of step (i) to a first media which includes a tissue organization promoting hormone; and (iii) subculturing said transformed plant cells after step (ii) onto a second media, to allow for shoot elongation, root development or both.

In any of the preceding methods or any other embodiments of methods of the present disclosure, the at least one agronomic characteristic may be selected from the group consisting of abiotic stress tolerance, nitrogen stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, whole plant amino acid content, vegetative tissue free amino acid content, fruit free amino acid content, seed free amino acid content, total plant protein content, fruit protein content, seed protein content, protein content in a vegetative tissue, drought tolerance, nitrogen uptake, resistance to root lodging, root architecture, root biomass, average root length, harvest index, stalk lodging, plant height, ear height, ear length, salt tolerance, early seedling vigor, and seedling emergence under low temperature stress. The alteration of at least one agronomic characteristic may be an increase in yield, greenness, or biomass.

In any of the preceding methods or any other embodiments of methods of the present disclosure, the plant may exhibit the alteration of at least one agronomic characteristic when compared, under nitrogen stress conditions, to a control plant not comprising said recombinant DNA construct (or suppression DNA construct).

In any of the preceding methods or any other embodiments, the altered root architecture may be an increase in root biomass or in average root length, or both.

In any of the preceding methods or any other embodiments of methods of the present disclosure, alternatives exist for introducing into a regenerable plant cell a recombinant DNA construct comprising a polynucleotide operably linked to at least one heterologous regulatory sequence. For example, one may introduce into a regenerable plant cell a regulatory sequence (such as one or more enhancers, optionally as part of a transposable element), and then screen for an event in which the regulatory sequence is operably linked to an endogenous gene encoding a polypeptide of the instant disclosure.

The introduction of recombinant DNA constructs of the present disclosure into plants may be carried out by any suitable technique, including but not limited to direct DNA uptake, chemical treatment, electroporation, microinjection, cell fusion, infection, vector mediated DNA transfer, bombardment, or Agrobacterium mediated transformation. Techniques for plant transformation and regeneration have been described in International Patent Publication WO 2009/006276, the contents of which are herein incorporated by reference.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

### EXAMPLES

The present disclosure is further illustrated in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Furthermore, various modifications of the disclosure in addition to those shown and described herein will be apparent to those skilled in the art from the foregoing description. Such modifications are also intended to fall within the scope of the appended claims.

### Example 1

### Creation of an Arabidopsis Population with Activation-Tagged Genes

An 18.49-kb T-DNA based binary construct was created, pHSbarENDs2 (PCT Publication No. WO/2012/058528), that contains four multimerized enhancer elements derived from the Cauliflower Mosaic Virus 35S promoter (corresponding to sequences −341 to −64, as defined by Odell et al., Nature 313:810-812 (1985)). The construct also contains vector sequences (pUC9) and a poly-linker (SEQ ID NO:1) to allow plasmid rescue, transposon sequences (Ds) to remobilize the T-DNA, and the bar gene to allow for glufosinate selection of transgenic plants. In principle, only the 10.8-kb segment from the right border (RB) to left border (LB) inclusive will be transferred into the host plant genome. Since the enhancer elements are located near the RB, they can induce cis-activation of genomic loci following T-DNA integration.

Arabidopsis activation-tagged populations were created by whole plant Agrobacterium transformation. The pHSbarENDs2 construct was transformed into Agrobacterium tumefaciens strain C58, grown in lysogeny broth medium at 25° C. to OD600 ˜1.0. Cells were then pelleted by centrifugation and resuspended in an equal volume of 5% sucrose/0.05% Silwet L-77 (OSI Specialties, Inc). At early bolting, soil grown Arabidopsis thaliana ecotype Col-0 were top watered with the Agrobacterium suspension. A week later, the same plants were top watered again with the same Agrobacterium strain in sucrose/Silwet. The plants were then allowed to set seed as normal. The resulting T1 seed were sown on soil, and transgenic seedlings were selected by spraying with glufosinate (FINALE® herbicide; AgrEvo; Bayer Environmental Science). A total of 100,000 glufosinate resistant T1 seedlings were selected. T2 seed from each line was kept separate.

### Example 2

### Screens to Identify Lines with Tolerance to Low Nitrogen

From each of 100,000 separate T1 activation-tagged lines, eleven T2 plants are sown on square plates (15 mm×15 mm) containing 0.5× N-Free Hoagland's, 0.4 mM potassium nitrate, 0.1% sucrose, 1 mM MES and 0.25% PHYTAGEL™ (Low N medium). Five lines are plated per plate, and the inclusion of 9 wild-type individuals on each plate makes for a total of 64 individuals in an 8×8 grid pattern (see FIG. 10). Plates are kept for three days in the dark at 4° C. to stratify seeds and then placed horizontally for nine days at 22° C. light and 20° C. dark. Photoperiod is sixteen hours light and eight hours dark, with an average light intensity of ˜200 mmol/m²/s. Plates are rotated and shuffled daily within each shelf. At day twelve (nine days of growth), seedling status is evaluated by imaging the entire plate.

After masking the plate image to remove background color, two different measurements are collected for each individual: total rosette area, and the percentage of color that falls into a green color bin. Using hue, saturation and intensity data (HSI), the green color bin consists of hues 50 to 66. Total rosette area is used as a measure of plant biomass, whereas the green color bin has been shown by dose-response studies to be an indicator of nitrogen assimilation.

Lines with a significant increase in total rosette area and/or green color bin, when compared to the wild-type controls, are designated as Phase 1 hits. Phase 1 hits are re-screened in duplicate under the same assay conditions (Phase 2 screen). A Phase 3 screen is also employed to further validate mutants that pass through Phases 1 and 2. In Phase 3, each line is plated separately on Low N medium, such that 32 T2 individuals are grown next to 32 wild-type individuals on one plate, providing greater statistical rigor to the analysis. If a line shows a significant difference from the controls in Phase 3, the line is then considered a validated nitrogen-deficiency tolerant line.

### Example 3

### Identification of Activation-Tagged Genes

Genes flanking the T-DNA insert in nitrogen tolerant lines are identified using one, or both, of the following two standard procedures: (1) thermal asymmetric interlaced (TAIL) PCR (Liu et al., Plant J. 8:457-63 (1995)); and (2) SAIFF PCR (Siebert et al., Nucleic Acids Res. 23:1087-1088 (1995)). In lines with complex multimerized T-DNA inserts, TAIL PCR and SAIFF PCR may both prove insufficient to identify candidate genes. In these cases, other procedures, including inverse PCR, plasmid rescue and/or genomic library construction, can be employed.

A successful result is one where a single TAIL or SAIFF PCR fragment contains a T-DNA border sequence and Arabidopsis genomic sequence. Once a tag of genomic sequence flanking a T-DNA insert is obtained, candidate genes are identified by alignment to publicly available Arabidopsis genome sequence. Specifically, the annotated gene nearest the 35S enhancer elements/T-DNA RB are candidates for genes that are activated.

To verify that an identified gene is truly near a T-DNA and to rule out the possibility that the TAIL/SAIFF fragment is a chimeric cloning artifact, a diagnostic PCR on genomic DNA is done with one oligo in the T-DNA and one oligo specific for the candidate gene. Genomic DNA samples that give a PCR product are interpreted as representing a T-DNA insertion. This analysis also verifies a situation in which more than one insertion event occurs in the same line, e.g., if multiple differing genomic fragments are identified in TAIL and/or SAIFF PCR analyses.

### Example 4A

### Identification of Activation-Tagged PH11 Gene

An activation tagged-line (line 101057) was further analyzed. DNA from the line was extracted, and genes flanking the T-DNA insert in the mutant line were identified using ligation-mediated PCR (Siebert et al., Nucleic Acids Res. 23:1087-1088 (1995)). A single amplified fragment was identified that contained a T-DNA border sequence and Arabidopsis genomic sequence. Once a tag of genomic sequence flanking a T-DNA insert was obtained, a candidate gene was identified by alignment to the completed Arabidopsis genome. Specifically, the annotated gene that was next to the nearest annotated gene to the 35S enhancer elements/T-DNA RB was the candidate for the gene activated in the line was also tested for validation. In the case of line 101057 the gene that was next to the nearest annotated gene to the 35S enhancer elements/T-DNA RB was At5g43870 (SEQ ID NO:2; NCBI GI No: 186529090), encoding the Arabidopsis thaliana “PH11” polypeptide referred to herein as AT-PH11 polypeptide (SEQ ID NO:3; NCBI GI 15240022).

### Example 4B

### Identification of Activation-Tagged NUCPU29 Gene

An activation tagged-line (line 119162) was further analyzed. DNA from the line was extracted, and genes flanking the T-DNA insert in the mutant line were identified using ligation-mediated PCR (Siebert et al., Nucleic Acids Res. 23:1087-1088 (1995)). A single amplified fragment was identified that contained a T-DNA border sequence and Arabidopsis genomic sequence. Once a tag of genomic sequence flanking a T-DNA insert was obtained, a candidate gene was identified by alignment to the completed Arabidopsis genome. Specifically, the annotated gene that was the nearest annotated gene to the 35S enhancer elements/T-DNA was the candidate for the gene activated in the line was also tested for validation. In the case of line 119162 the gene nearest the 35S enhancer was At2g36030 (SEQ ID NO:69; NCBI GI No: 18404023), encoding the Arabidopsis thaliana “NUCPU29” polypeptide referred to herein as AT-NUCPU29 (SEQ ID NO:70; NCBI GI 15227560).

### Example 5A

### Validation of Candidate Arabidopsis Gene (At5g43870) Via Transformation into Arabidopsis

Candidate genes can be transformed into Arabidopsis and overexpressed under the 35S promoter. If the same or similar phenotype is observed in the transgenic line as in the parent activation-tagged line, then the candidate gene is considered to be a validated “lead gene” in Arabidopsis.

The candidate Arabidopsis PH11 gene (At5g43870; SEQ ID NO:2; NCBI GI No. 186529090), encoding a PH11 polypeptide, was tested for its ability to confer nitrogen-deficiency tolerance in the following manner.

The At5g43870 CDS was synthesized and cloned into pDONR™ Zeo.

Using the INVITROGEN™ GATEWAY® CLONASE™ technology, a BP Recombination Reaction was performed for the PCR product with pDONR™ Zeo (PCT Publication No. WO/2012/058528). This process removes the bacteria lethal ccdB gene, as well as the chloramphenicol resistance gene (CAM) from pDONR™ Zeo and directionally clones the PCR product with flanking attB1 and attB2 sites, creating an entry clone. This entry clone was used for a subsequent LR Recombination Reaction with a destination vector, as follows.

A 16.8-kb T-DNA based binary vector (destination vector), called pBC-yellow (PCT Publication No. WO/2012/058528), was constructed with a 1.3-kb 35S promoter immediately upstream of the INVITROGEN™ GATEWAY C1 conversion insert, which contains the bacterial lethal ccdB gene as well as the chloramphenicol resistance gene (CAM) flanked by attR1 and attR2 sequences. The vector also contains the RD29a promoter driving expression of the gene for ZS-Yellow (INVITROGEN™), which confers yellow fluorescence to transformed seed. Using the INVITROGEN™ GATEWAY® technology, an LR Recombination Reaction was performed on the entry clone containing the synthesized At5g43870 CDS product and pBC-yellow. This amplification allowed for rapid and directional cloning of the At5g43870 gene behind the 35S promoter in pBC-yellow.

Applicants then introduced the 35S promoter::At5g43870 expression construct into wild-type Arabidopsis ecotype Col-0, using the same Agrobacterium-mediated transformation procedure described in Example 1. Transgenic T1 seeds were selected by yellow fluorescence, and 32 of these T1 seeds were plated next to 32 T1 empty vector transformed wild-type Arabidopsis ecotype Col-0 seeds selected by yellow fluorescence on low nitrogen medium. All subsequent growth conditions and imaging analyses were performed as described in Example 2. It was found that the wild-type Arabidopsis plants that were transformed with a construct where At5g43870 was directly expressed by the 35S promoter exhibited tolerance to nitrogen limiting conditions. The assay involved the comparison of 32 transgenic seedlings with 32 control seedlings for size and green color on 4 consecutive days.

P value of less than 10⁻³ for either of the parameters in imaging done on multiple days is considered a validation.

The At5g43870 CDS was also cloned under the root-specific PHT1 promoter (SEQ ID NO:64), shoot-specific OEE-2 promoter (SEQ ID NO:65) and vasculature-specific SUC2 promoter (SEQ ID NO:66). Positive validation in a lowN assay was obtained with the 35S promoter. Some negative phenotypes were seen with the PHT1 and the SUC2 promoters.

The validation scores (P-value) for AT-PH11 using the 35S promoter were found to be 5.70E-04 for area, and 6.12E-5 for bin2.

### Example 5B

### Validation of Candidate Arabidopsis Gene (At2g36030) Via Transformation into Arabidopsis

The candidate Arabidopsis NUCPU29 gene (At2g36030; SEQ ID NO:69; NCBI GI No. 18404023), encoding a NUCPU29 polypeptide, was tested for its ability to confer nitrogen-deficiency tolerance in the following manner.

The At2g36030 genomic DNA (SEQ ID NO:69) was amplified by PCR with the following primers:

1. At2g36030-5′ attB forward primer (SEQ ID NO:67)

The forward primer contains the attB1 sequence (ACAAGTTTGTACAAAAAAGCAGGCT; SEQ ID NO:72) and a consensus Kozak sequence (CAACA) upstream of the 21 nucleotides starting from the ATG start codon, of said cDNA.

2. At2g36030-3′ attB reverse primer (SEQ ID NO:68)

The reverse primer contains the attB2 sequence (ACCACTTTGTACAAGAAAGCTGGGT; SEQ ID NO:73) adjacent to the reverse complement of 21 nucleotides from the stop codon, of said cDNA.

Using the INVITROGEN™ GATEWAY® CLONASE™ technology, a BP Recombination Reaction was performed for the PCR product with pDONR™ Zeo (PCT Publication No. WO/2012/058528). This process removes the bacteria lethal ccdB gene, as well as the chloramphenicol resistance gene (CAM) from pDONR™ Zeo and directionally clones the PCR product with flanking attB1 and attB2 sites, creating an entry clone. This entry clone was used for a subsequent LR Recombination Reaction with a destination vector, as follows.

At2g36030 gene was cloned behind the 35S promoter in pBC-yellow, as described in Example 5A.

Applicants then introduced the 35S promoter::At2g36030 expression construct into wild-type Arabidopsis ecotype Col-0, using the same Agrobacterium-mediated transformation procedure described in Example 1. Transgenic T1 seeds were selected by yellow fluorescence, and 32 of these T1 seeds were plated next to 32 T1 empty vector transformed wild-type Arabidopsis ecotype Col-0 seeds selected by yellow fluorescence on low nitrogen medium. All subsequent growth conditions and imaging analyses were performed as described in Example 2. It was found that the wild-type Arabidopsis plants that were transformed with a construct where At2g36030 was directly expressed by the 35S promoter exhibited tolerance to nitrogen limiting conditions. The assay involved the comparison of 32 transgenic seedlings with 32 control seedlings for size and green color on 4 consecutive days.

P value of less than 10⁻³ for either of the parameters for any of the days is considered a validation.

The At2g36030 CDS (SEQ ID NO:69) was also cloned under the root specific PHT1 promoter (SEQ ID NO:64), shoot-specific OEE-2 promoter (SEQ ID NO:65) and vasculature-specific SUC2 promoter (SEQ ID NO:66). Positive validation in a lowN assay was obtained with the root-specific and vasculature-specific promoter.

The validation scores (P-value) for AT-NUCPU29 using different promoters are given in Table 2. The values given in bold are positive validations.

TABLE 2

Parameter

| Promoter | Area | bin2 |
|---|---|---|
| 35S | 5.87E−12 | 4.19E−02 |
| OEE2 | 8.84E−01 | 5.27E−01 |
| PHT1 | 3.04E−06 | 9.65E−01 |
| SUC2 | 8.02E−10 | 1.32E−04 |

### Example 6A

### Preparation of cDNA Libraries and Isolation and Sequencing of cDNA Clones

cDNA libraries may be prepared by any one of many methods available. For example, the cDNAs may be introduced into plasmid vectors by first preparing the cDNA libraries in UNI-ZAP™ XR vectors according to the manufacturer's protocol (Stratagene Cloning Systems, La Jolla, Calif.). The UNI-ZAP™ XR libraries are converted into plasmid libraries according to the protocol provided by Stratagene. Upon conversion, cDNA inserts will be contained in the plasmid vector pBLUESCRIPT®. In addition, the cDNAs may be introduced directly into precut BLUESCRIPT® II SK(+) vectors (Stratagene) using T4 DNA ligase (New England Biolabs), followed by transfection into DH10B cells according to the manufacturer's protocol (GIBCO BRL Products). Once the cDNA inserts are in plasmid vectors, plasmid DNAs are prepared from randomly picked bacterial colonies containing recombinant pBLUESCRIPT® plasmids, or the insert cDNA sequences are amplified via polymerase chain reaction using primers specific for vector sequences flanking the inserted cDNA sequences. Amplified insert DNAs or plasmid DNAs are sequenced in dye-primer sequencing reactions to generate partial cDNA sequences (expressed sequence tags or “ESTs”; see Adams et al., Science 252:1651-1656 (1991)). The resulting ESTs are analyzed using a Perkin Elmer Model 377 fluorescent sequencer.

Full-insert sequence (FIS) data is generated utilizing a modified transposition protocol. Clones identified for FIS are recovered from archived glycerol stocks as single colonies, and plasmid DNAs are isolated via alkaline lysis. Isolated DNA templates are reacted with vector primed M13 forward and reverse oligonucleotides in a PCR-based sequencing reaction and loaded onto automated sequencers. Confirmation of clone identification is performed by sequence alignment to the original EST sequence from which the FIS request is made.

Confirmed templates are transposed via the Primer Island transposition kit (PE Applied Biosystems, Foster City, Calif.) which is based upon the Saccharomyces cerevisiae Ty1 transposable element (Devine and Boeke, Nucleic Acids Res. 22:3765-3772 (1994)). The in vitro transposition system places unique binding sites randomly throughout a population of large DNA molecules. The transposed DNA is then used to transform DH10B electro-competent cells (GIBCO BRL/Life Technologies, Rockville, Md.) via electroporation. The transposable element contains an additional selectable marker (named DHFR; Fling and Richards, Nucleic Acids Res. 11:5147-5158 (1983)), allowing for dual selection on agar plates of only those subclones containing the integrated transposon. Multiple subclones are randomly selected from each transposition reaction, plasmid DNAs are prepared via alkaline lysis, and templates are sequenced (ABI PRISM dye-terminator ReadyReaction mix) outward from the transposition event site, utilizing unique primers specific to the binding sites within the transposon.

Sequence data is collected (ABI PRISM® Collections) and assembled using Phred and Phrap (Ewing et al., Genome Res. 8:175-185 (1998); Ewing et al., Genome Res. 8:186-194 (1998)). Phred is a public domain software program which re-reads the ABI sequence data, re-calls the bases, assigns quality values, and writes the base calls and quality values into editable output files. The Phrap sequence assembly program uses these quality values to increase the accuracy of the assembled sequence contigs. Assemblies are viewed by the Consed sequence editor (Gordon et al., Genome Res. 8:195-202 (1998)).

In some of the clones the cDNA fragment corresponds to a portion of the 3′-terminus of the gene and does not cover the entire open reading frame. In order to obtain the upstream information one of two different protocols is used. The first of these methods results in the production of a fragment of DNA containing a portion of the desired gene sequence while the second method results in the production of a fragment containing the entire open reading frame. Both of these methods use two rounds of PCR amplification to obtain fragments from one or more libraries. The libraries sometimes are chosen based on previous knowledge that the specific gene should be found in a certain tissue and sometimes are randomly-chosen. Reactions to obtain the same gene may be performed on several libraries in parallel or on a pool of libraries. Library pools are normally prepared using from 3 to 5 different libraries and normalized to a uniform dilution. In the first round of amplification both methods use a vector-specific (forward) primer corresponding to a portion of the vector located at the 5′-terminus of the clone coupled with a gene-specific (reverse) primer. The first method uses a sequence that is complementary to a portion of the already known gene sequence while the second method uses a gene-specific primer complementary to a portion of the 3′-untranslated region (also referred to as UTR). In the second round of amplification a nested set of primers is used for both methods. The resulting DNA fragment is ligated into a pBLUESCRIPT® vector using a commercial kit and following the manufacturer's protocol. This kit is selected from many available from several vendors including INVITROGEN™ (Carlsbad, Calif.), Promega Biotech (Madison, Wis.), and GIBCO-BRL (Gaithersburg, Md.). The plasmid DNA is isolated by alkaline lysis method and submitted for sequencing and assembly using Phred/Phrap, as above.

### Example 6B

### cDNA Indexing and Assembly

Plants were treated either with drought or with nitrogen deficiency. Aerial and root tissues were harvested separately. Total RNA was then extracted separately from leaf and root tissues and subsequently polyA RNA was purified. PolyA RNAs were subjected to RNA ligase-mediated full-length transcript enrichment method. First strand cDNAs were synthesized using SUPERSCRIPT® III with barcoded 3′ primers which differentiate transcripts source (root vs aerial). These cDNAs were then combined and subjected to second strand synthesis. Normalization of cDNAs was performed using a modified EVROGEN® protocol. After Sfil digestion and size fractionation, cDNAs were cloned into pENTR™-Sfil vector. For indexing process, independent clones were arrayed into 384 well plates, a total of 260 plates per library. Each clone was then PCR amplified, using barcode primers. 1,536 clones having unique barcodes in their 5′ and 3′ ends were pooled as one sample and processed to have one ILLUMINA® tag. A total of 8 samples (12,288 clones) were loaded onto one lane of Hi-seq sequencer and sequenced.

Assembly: Reads from the sequencing runs can be soft-trimmed prior to assembly such that the first base pair of each read with an observed FASTQ quality score lower than 15 and all subsequent bases are clipped using a Python script. The Velvet assembler (Zerbino et al. (2008) Genome Research 18:821-829) can be run under varying kmer and coverage cutoff parameters to produce several putative assemblies along a range of stringency. The contiguous sequences (contigs) within those assemblies can be combined into clusters using Vmatch software (available on the Vmatch website) such that contigs which are identified as substrings of longer contigs are grouped and eliminated, leaving a non-redundant set of longest “sentinel” contigs. These non-redundant sets can be used in alignments to homologous sequences from known model plant species.

Bar-coded reads can be extended from both ends of a clone with the SSAKE assembler (Warren et al. Bioinformatics. 23:500-501 2007). End sequences of clones that do not assemble to completion with SSAKE can be matched to each other with the CAP3 assembler (Huang X et al. (1999) Genome Res. 9:868-877) at high stringency, with the resulting matches forming a complete contig. End sequences from clones that still fail to assemble completely are aligned with Velvet contigs from the bulk assembly at a high homology setting with BLAST, assembly of the resulting matches can be contiged with CAP3, or with the Minimo component of the AMOS package (Treangen et al. (2011) Curr Protoc Bioinformatics Unit 11.8). Clone ends that fail to complete are stored as incomplete assemblies.

### Example 7

### Identification of cDNA Clones

cDNA clones encoding PH11 or NUCPU29 polypeptides may be identified by conducting BLAST (Basic Local Alignment Search Tool; Altschul et al., J. Mol. Biol. 215:403-410 (1993); see also the explanation of the BLAST algorithm on the world wide web site for the National Center for Biotechnology Information at the National Library of Medicine of the National Institutes of Health) searches for similarity to amino acid sequences contained in the BLAST “nr” database (comprising all non-redundant GenBank CDS translations, sequences derived from the 3-dimensional structure Brookhaven Protein Data Bank, the last major release of the SWISS-PROT protein sequence database, EMBL, and DDBJ databases). The DNA sequences from clones can be translated in all reading frames and compared for similarity to all publicly available protein sequences contained in the “nr” database using the BLASTX algorithm (Gish and States, Nat. Genet. 3:266-272 (1993)) provided by the NCBI. Alternatively, the polypeptides encoded by the cDNA sequences can be analyzed for similarity to all publicly available amino acid sequences contained in the “nr” database using the BLASTP algorithm provided by the National Center for Biotechnology Information (NCBI). For convenience, the P-value (probability) or the E-value (expection) of observing a match of a cDNA-encoded sequence to a sequence contained in the searched databases merely by chance as calculated by BLAST are reported herein as “pLog” values, which represent the negative of the logarithm of the reported P-value or E-value. Accordingly, the greater the pLog value, the greater the likelihood that the cDNA-encoded sequence and the BLAST “hit” represent homologous proteins.

EST sequences can be compared to the GenBank database as described above. ESTs that contain sequences more 5- or 3-prime can be found by using the BLASTN algorithm (Altschul et al., Nucleic Acids Res. 25:3389-3402 (1997)) against the DUPONT proprietary database comparing nucleotide sequences that share common or overlapping regions of sequence homology. Where common or overlapping sequences exist between two or more nucleic acid fragments, the sequences can be assembled into a single contiguous nucleotide sequence, thus extending the original fragment in either the 5 or 3 prime direction. Once the most 5-prime EST is identified, its complete sequence can be determined by Full Insert Sequencing as described above.

Homologous genes belonging to different species can be found by comparing the amino acid sequence of a known gene (from either a proprietary source or a public database) against an EST database using the tBLASTn algorithm. The tBLASTn algorithm searches an amino acid query against a nucleotide database that is translated in all 6 reading frames. This search allows for differences in nucleotide codon usage between different species, and for codon degeneracy.

### Example 8A

### Characterization of cDNA Clones Encoding PH11 Polypeptides

cDNA libraries representing mRNAs from various tissues of maize, rice and soybean can be prepared and cDNA clones encoding PH11 polypeptides can be identified. PH11 polypeptides can also be identified from exotic plant species, Paspalum notatum, commonly called Bahia grass, Lamium amplexicaule, Artemisia tridentata, Sesbania bispinosa, Delosperma nubigenum. Mining of homologs from resurrection and Bahia grass can be performed by performing a TblastN of the Arabidopsis PH11 genes, and the identified maize PH11 homologs against the Bahia and resurrection grass assemblies. Homologs can also be identified by cDNA indexing and assembly. The resulting hits can be translated based on the blast alignments; and the translations can be aligned with the other known PH11 polypeptides.

Similarly cDNA clones encoding NUCPU29 polypeptides may be identified.

### Example 8B

### Identification of Other PH11 and NUCPU29 Polypeptides

Sequences homologous to the lead genes that encode the Arabidopsis thaliana AT-PH11 or AT-NUCPU29 polypeptides can be identified using sequence comparison algorithms such as BLAST (Basic Local Alignment Search Tool; Altschul et al., J. Mol. Biol. 215:403-410 (1993); see also the explanation of the BLAST algorithm on the world wide web site for the National Center for Biotechnology Information at the National Library of Medicine of the National Institutes of Health). Mining of PH11 and NUCPU29 nucleotide homologs from exotic species such as Bahia grass can also done by performing a TblastN of the Arabidopsis AT-PH11, (At5g43870; SEQ ID NO:2), and Arabidopsis AT-NUCPU29, (At2g36030; SEQ ID NO:69) against the Bahia assemblies. The resulting hits can be assembled in a desktop assembly program (DNASTAR® SEQMAN™) and the resulting contig can be translated based on the blast alignments, such that the assembled translation would return the correct protein in a single frame. These computer assemblies can then be aligned with the other sequences as above.

### Example 8C

### Sequence Alignment and Percent Identity Calculations for PH11 and NUCPU29 Polypeptides

Sequence alignments and percent identity calculations may be performed using the MEGALIGN® program of the LASERGENE® bioinformatics computing suite (DNASTAR Inc., Madison, Wis.). Multiple alignment of the sequences may be performed using the Clustal V method of alignment (Higgins and Sharp (1989) CABIOS. 5:151-153) with the default parameters (GAP PENALTY=10, GAP LENGTH PENALTY=10). Default parameters for pairwise alignments using the Clustal method are KTUPLE=1, GAP PENALTY=3, WINDOW=5 and DIAGONALS SAVED=5.

BLASTP can be done for all the PH11 and NUCPU29 homologs identified, which can be individual ESTs (“EST”), the sequences of the entire cDNA inserts comprising the indicated cDNA clones (“FIS”), the sequences of contigs assembled from two or more EST, FIS or PCR sequences (“Contig”), sequences encoding an entire or functional protein derived from an FIS or a contig (“CGS”), or sequences derived from full range genomic PCR capture and FGENESH analysis.

### Example 8D

### Other PH11 and NUCPU29 Polypeptides

cDNA libraries representing mRNAs from various tissues of Zea mays, Dennstaedtia punctilobula, Sesbania bispinosa, Artemisia tridentata, Lamium amplexicaule, Delosperma nubigenum, Peperomia caperata were prepared and cDNA clones encoding PH11 polypeptides were identified. The characteristics of the libraries are described below.

TABLE 3
Details of cDNA Libraries and Datasets

| Library or | | |
|---|---|---|
| Dataset | Description | Clone |
| dpzm | corn; proprietary dataset | ZM-PH11-A |
| dpzm | corn; proprietary dataset | ZM-PH11-B |
| dpzm | corn; proprietary dataset | ZM-PH11-C |
| dpzm | corn; proprietary dataset | ZM-PH11-D |
| dpzm | corn; proprietary dataset | ZM-PH11-E |
| sesgr1n | Sesbania bispinosa; root and leaf | sesgr1n.pk036.o8 |
| ahgr1c | Amaranthus hypochondriacus; root and | ahgr1c.pk088.o5 |
| | shoot | |
| arttr1n | Artemisia tridentata; root; Total RNA | arttr1n.pk119.a3 |
| icegr1n | Delosperma nubigenum; root and shoot | icegr1n.pk136.c5 |
| lpgr1n | Linum perenne v. lewisii (blue flax); root | lpgr1n.pk075.f12 |
| | and leaf | |
| lpgr1n | Linum perenne v. lewisii (blue flax); root | lpgr1n.pk026.f24 |
| | and leaf | |
| tmgr2n | Triglochin maritima; root and leaf | tmgr2n.pk006.j11 |
| tsgr1n | Tradescantia sillamontana; root, | tsgr1n.pk037.f8 |
| | stem, leaf tissue | |
| tsgr1n | Tradescantia sillamontana; root, stem, | tsgr1n.pk020.c8 |
| | leaf tissue | |

A BLAST search using the AT-PH11 polypeptide and maize sequences from clones listed in Table 3 revealed similarity of the polypeptides encoded by the cDNAs to the PH11 polypeptides from various organisms. As shown in Table 3 and FIG. 4A-FIG. 4K, certain cDNAs encoded polypeptides similar to PH11 polypeptide from Arabidopsis (GI No. 15240022; SEQ ID NO:3).

Shown in Table 4 (non-patent literature) and Table 5 (patent literature) are the BLAST results for one or more of the following: individual Expressed Sequence Tag (“EST”), the sequences of the entire cDNA inserts comprising the indicated cDNA clones (“Full-Insert Sequence” or “FIS”), the sequences of contigs assembled from two or more EST, FIS or PCR sequences (“Contig”), or sequences encoding an entire or functional protein derived from an FIS or a contig (“Complete Gene Sequence” or “CGS”). Also shown in Table 4 and 5 are the percent sequence identity values for each pair of amino acid sequences using the Clustal V method of alignment with default parameters.

TABLE 4
BLASTP Results for PH11 polypeptides

| | | BLASTP | Percent |
|---|---|---|---|
| Sequence | NCBI GI No. | pLog of | Sequence |
| (SEQ ID NO) | (SEQ ID NO) | E-value | Identity |
| ZM-PH11-A | 195656831 | >180 | 100 |
| (SEQ ID NO: 7) | (SEQ ID NO: 54) | | |
| ZM-PH11-B | 293336271 | >180 | 85.9 |
| (SEQ ID NO: 9) | (SEQ ID NO: 56) | | |
| ZM-PH11-C | 514754743 | >180 | 78.5 |
| (SEQ ID NO: 11) | (SEQ ID NO: 58) | | |
| ZM-PH11-D | 242055855 | >180 | 85.2 |
| (SEQ ID NO: 12) | (SEQ ID NO: 60) | | |
| ZM-PH11-E | K7UXW2 | >180 | 99.3 |
| (SEQ ID NO: 15) | (SEQ ID NO: 62) | | |

TABLE 5
BLASTP Results for PH11 polypeptides

| | | BLASTP | Percent |
|---|---|---|---|
| Sequence | Reference | pLog of | Sequence |
| (SEQ ID NO) | (SEQ ID NO) | E-value | Identity |
| At5g43870 | SEQ ID NO: 1647 of | >180 | 100 |
| (SEQ ID NO: 3) | US20120227134 | | |
| | (SEQ ID NO: 52) | | |
| ZM-PH11-A | SEQ ID NO: 1566 of | >180 | 100 |
| (SEQ ID NO: 7) | US20120096584 | | |
| | (SEQ ID NO: 53) | | |
| ZM-PH11-B | SEQ ID NO: 68801 of | >180 | 71.3 |
| (SEQ ID NO: 9) | US20120017338 | | |
| | (SEQ ID NO: 55) | | |
| ZM-PH11-C | SEQ ID NO: 58064 of | >180 | 100 |
| (SEQ ID NO: 11) | US20120017292 | | |
| | (SEQ ID NO: 57) | | |
| ZM-PH11-D | SEQ ID NO: 43844 of | >180 | 100 |
| (SEQ ID NO: 12) | US20120017338 | | |
| | (SEQ ID NO: 59) | | |
| ZM-PH11-E | SEQ ID NO: 174 of | >180 | 100 |
| (SEQ ID NO: 15) | WO2013072832 | | |
| | (SEQ ID NO: 64) | | |

FIGS. 4A-4K show the alignment of the PH11 polypeptides from different plant species. The alignment of SEQ ID NOS. 3, 7, 9, 11, 13, 15, 17, 19, 21, 23, 25, 27, 29, 31, 33, 52-61 and 62 is shown. Residues that are identical to the residue of the AT-PH11 sequence (SEQ ID NO:3) at a given position are enclosed in a box. A consensus sequence (SEQ ID NO:63) is presented where a residue is shown if present in majority of sequences, otherwise, a period is shown.

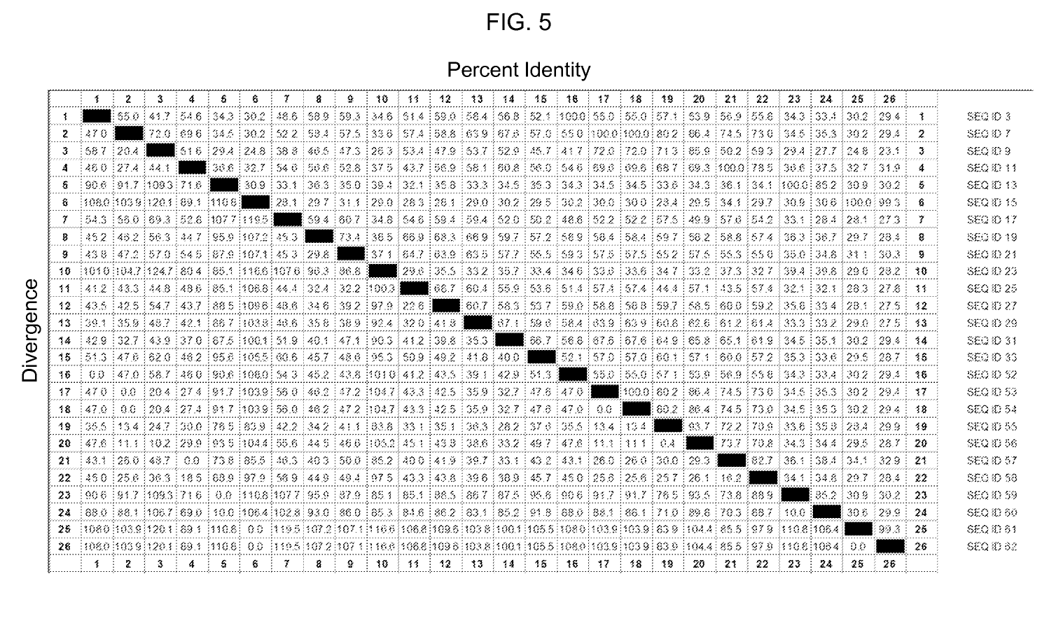

FIG. 5 shows the percent sequence identity and the divergence values for each pair of amino acids sequences of PH11 polypeptides displayed in FIG. 4A-4K.

### Example 9

### Preparation of a Plant Expression Vector Containing a Homolog to the Arabidopsis Lead Gene

Sequences homologous to the Arabidopsis PH11 and NUCPU29 gene can be identified using sequence comparison algorithms such as BLAST (Basic Local Alignment Search Tool; Altschul et al., J. Mol. Biol. 215:403-410 (1993); see also the explanation of the BLAST algorithm on the world wide web site for the National Center for Biotechnology Information at the National Library of Medicine of the National Institutes of Health). Sequences encoding PH11 and NUCPU29 polypeptides can be PCR-amplified by any of the following methods.

Method 1 (RNA-based): If the 5′ and 3′ sequence information for the protein-coding region of a PH11 PH11 homolog is available, gene-specific primers can be designed as outlined in Example 5. RT-PCR can be used with plant RNA to obtain a nucleic acid fragment containing the protein-coding region flanked by attB1 and attB2 sequences (PCT Publication No. WO/2012/058528). The primer may contain a consensus Kozak sequence (CAACA) upstream of the start codon.

Method 2 (DNA-based): Alternatively, if a cDNA clone is available for the PH11 homolog, the entire cDNA insert (containing 5′ and 3′ non-coding regions) can be PCR amplified. Forward and reverse primers can be designed that contain either the attB1 sequence and vector-specific sequence that precedes the cDNA insert or the attB2 sequence and vector-specific sequence that follows the cDNA insert, respectively. For a cDNA insert cloned into the vector pBLUESCRIPT SK+, the forward primer VC062 (PCT Publication No. WO/2012/058528) and the reverse primer VC063 (PCT Publication No. WO/2012/058528) can be used.

Method 3 (genomic DNA): Genomic sequences and clones can be obtained by long range genomic PCR capture using a high-fidelity DNA polymerase. Primers can be designed based on the sequences in the 5′ and 3′ regions of the genomic locus and the resulting PCR product can be sequenced and cloned by ligation to a blunt-ended, linearized GATEWAY® entry vector. The sequence can be analyzed using the FGENESH program (Salamov, A. and Solovyev, V. (2000) Genome Res., 10: 516-522), and optionally, can be aligned with homologous sequences from other species to assist in identification of putative introns and exons.

Methods 1, 2, and 3 can be modified according to procedures known by one skilled in the art. For example, the primers of Method 1 may contain restriction sites instead of attB1 and attB2 sites, for subsequent cloning of the PCR product into a vector containing attB1 and attB2 sites. Additionally, Method 2 can involve amplification from a cDNA clone, a lambda clone, a BAC clone or genomic DNA.

A PCR product obtained by any method above can be combined with a GATEWAY® donor vector, such as pDONR™ Zeo (PCT Publication No. WO/2012/058528) or pDONR™221 (PCT Publication No. WO/2012/058528), using a BP Recombination Reaction. This process removes the bacteria lethal ccdB gene, as well as the chloramphenicol resistance gene (CAM), from pDONR™ Zeo or pDONR™221 and directionally clones the PCR product with flanking attB1 and attB2 sites to create an entry clone. Using the INVITROGEN™ GATEWAY® CLONASE™ technology, the sequence encoding the homologous PH11 or NUCPU29 polypeptide from the entry clone can then be transferred to a suitable destination vector, such as pBC-Yellow (PCT Publication No. WO/2012/058528), PHP27840 (PCT Publication No. WO/2012/058528), or PHP23236 (PCT Publication No. WO/2012/058528), to obtain a plant expression vector for use with Arabidopsis, soybean, and corn, respectively.

A BP Reaction is a recombination reaction between an Expression Clone (or an attB-flanked PCR product) and a Donor (e.g., pDONR™) Vector to create an Entry Clone. A LR Reaction is a recombination between an Entry Clone and a Destination Vector to create an Expression Clone. A Donor Vector contains attP1 and attP2 sites. An Entry Clone contains attL1 and attL2 sites. A Destination Vector contains attR1 and attR2 site. An Expression Clone contains attB1 and attB2 sites. The attB1 site is composed of parts of the attL1 and attR1 sites. The attB2 site is composed of parts of the attL2 and attR2 sites.

Alternatively a MultiSite GATEWAY® LR recombination reaction between multiple entry clones and a suitable destination vector can be performed to create an expression vector.

### Example 10

### Preparation of Soybean Expression Vectors and Transformation of Soybean with Validated Arabidopsis Lead Genes

Soybean plants can be transformed to overexpress each validated Arabidopsis gene or the corresponding homologs from various species in order to examine the resulting phenotype.

The same GATEWAY® entry clone described in Example 5 can be used to directionally clone each gene into the PHP27840 vector such that expression of the gene is under control of the SCP1 promoter.

Soybean embryos may then be transformed with the expression vector comprising sequences encoding the instant polypeptides. Techniques for soybean transformation and regeneration have been described in International Patent Publication WO 2009/006276, the contents of which are herein incorporated by reference.

T1 plants can be grown under nitrogen limiting conditions, for example, 1 mM nitrate, and analyzed for phenotypic changes. The following parameters can be quantified using image analysis: plant area, volume, growth rate and color analysis can be collected and quantified. Overexpression constructs that result in an alteration, compared to suitable control plants, in greenness (green color bin), yield, growth rate, biomass, fresh or dry weight at maturation, fruit or seed yield, total plant nitrogen content, fruit or seed nitrogen content, nitrogen content in vegetative tissue, free amino acid content in the whole plant, free amino acid content in vegetative tissue, free amino acid content in the fruit or seed, protein content in the fruit or seed, or protein content in a vegetative tissue can be considered evidence that the Arabidopsis lead gene functions in soybean to enhance tolerance to nitrogen deprivation (increased nitrogen tolerance).

Soybean plants transformed with validated genes can be assayed to study agronomic characteristics relative to control or reference plants. For example, yield enhancement and/or stability under low and high nitrogen conditions (e.g., nitrogen limiting conditions and nitrogen-sufficient conditions) can be assayed.

### Example 11

### Transformation of Maize with Validated Arabidopsis Lead Genes Using Particle Bombardment

Maize plants can be transformed to overexpress a validated Arabidopsis lead gene or the corresponding homologs from various species in order to examine the resulting phenotype.

The same GATEWAY® entry clone described in Example 5 can be used to directionally clone each gene into a maize transformation vector. Expression of the gene in the maize transformation vector can be under control of a constitutive promoter such as the maize ubiquitin promoter (Christensen et al., Plant Mol. Biol. 12:619-632 (1989) and Christensen et al., Plant Mol. Biol. 18:675-689 (1992))

The recombinant DNA construct described above can then be introduced into maize cells by particle bombardment. Techniques for corn transformation by particle bombardment have been described in International Patent Publication WO 2009/006276, the contents of which are herein incorporated by reference.

T1 plants can be grown under nitrogen limiting conditions, for example 1 mM nitrate, and analyzed for phenotypic changes. The following parameters can be quantified using image analysis: plant area, volume, growth rate and color analysis can be collected and quantified. Overexpression constructs that result in an alteration, compared to suitable control plants, in greenness (green color bin), yield, growth rate, biomass, fresh or dry weight at maturation, fruit or seed yield, total plant nitrogen content, fruit or seed nitrogen content, nitrogen content in vegetative tissue, free amino acid content in the whole plant, free amino acid content in vegetative tissue, free amino acid content in the fruit or seed, protein content in the fruit or seed, or protein content in a vegetative tissue can be considered evidence that the Arabidopsis lead gene functions in maize to enhance tolerance to nitrogen deprivation (increased nitrogen tolerance).

### Example 12

### Electroporation of Agrobacterium tumefaciens LBA4404

Electroporation competent cells (40 μL), such as Agrobacterium tumefaciens LBA4404 (containing PHP10523), are thawed on ice (20-30 min). PHP10523 contains VIR genes for T-DNA transfer, an Agrobacterium low copy number plasmid origin of replication, a tetracycline resistance gene, and a Cos site for in vivo DNA bimolecular recombination. Meanwhile the electroporation cuvette is chilled on ice. The electroporator settings are adjusted to 2.1 kV. A DNA aliquot (0.5 μL parental DNA at a concentration of 0.2 μg-1.0 μg in low salt buffer or twice distilled H₂O) is mixed with the thawed Agrobacterium tumefaciens LBA4404 cells while still on ice. The mixture is transferred to the bottom of electroporation cuvette and kept at rest on ice for 1-2 min. The cells are electroporated (Eppendorf electroporator 2510) by pushing the “pulse” button twice (ideally achieving a 4.0 millisecond pulse). Subsequently, 0.5 mL of room temperature 2xYT medium (or SOC medium) are added to the cuvette and transferred to a 15 mL snap-cap tube (e.g., FALCON™ tube). The cells are incubated at 28-30° C., 200-250 rpm for 3 h.

Aliquots of 250 μL are spread onto plates containing YM medium and 50 μg/mL spectinomycin and incubated three days at 28-30° C. To increase the number of transformants one of two optional steps can be performed:

Option 1: Overlay plates with 30 μL of 15 mg/mL rifampicin. LBA4404 has a chromosomal resistance gene for rifampicin. This additional selection eliminates some contaminating colonies observed when using poorer preparations of LBA4404 competent cells.

Option 2: Perform two replicates of the electroporation to compensate for poorer electrocompetent cells.

### Identification of Transformants:

Four independent colonies are picked and streaked on plates containing AB minimal medium and 50 μg/mL spectinomycin for isolation of single colonies. The plates are incubated at 28° C. for two to three days. A single colony for each putative cointegrate is picked and inoculated with 4 mL of 10 g/L bactopeptone, 10 g/L yeast extract, 5 g/L sodium chloride, and 50 mg/L spectinomycin. The mixture is incubated for 24 h at 28° C. with shaking. Plasmid DNA from 4 mL of culture is isolated using QIAGEN Miniprep and an optional Buffer PB wash. The DNA is eluted in 30 μL. Aliquots of 2 μL are used to electroporate 20 μL of DH10b+20 μL of twice distilled H₂O as per above. Optionally a 15 μL aliquot can be used to transform 75-100 μl_ of INVITROGEN™ Library Efficiency DH5a. The cells are spread on plates containing LB medium and 50 μg/mL spectinomycin and incubated at 37° C. overnight.

Three to four independent colonies are picked for each putative cointegrate and inoculated 4 mL of 2xYT medium (10 g/L bactopeptone, 10 g/L yeast extract, 5 g/L sodium chloride) with 50 μg/mL spectinomycin. The cells are incubated at 37° C. overnight with shaking. Next, plasmid DNA is isolated from 4 mL of culture using QIAPREP® Miniprep with optional Buffer PB wash (elute in 50 μL). 8 μL are used for digestion with SalI (using parental DNA and PHP10523 as controls). Three more digestions using restriction enzymes BamHI, EcoRI, and HindIII are performed for 4 plasmids that represent 2 putative cointegrates with correct SalI digestion pattern (using parental DNA and PHP10523 as controls). Electronic gels are recommended for comparison.

### Example 13

### Transformation of Maize Using Agrobacterium

Maize plants can be transformed to overexpress a validated Arabidopsis lead gene or the corresponding homologs from various species in order to examine the resulting phenotype.

Agrobacterium-mediated transformation of maize is performed essentially as described by Zhao et al., in Meth. Mol. Biol. 318:315-323 (2006) (see also Zhao et al., Mol. Breed. 8:323-333 (2001) and U.S. Pat. No. 5,981,840 issued Nov. 9, 1999, incorporated herein by reference). The transformation process involves bacterium inoculation, co-cultivation, resting, selection, and plant regeneration.

### 1. Immature Embryo Preparation:

Immature maize embryos are dissected from caryopses and placed in a 2 mL microtube containing 2 mL PHI-A medium.

### 2. Agrobacterium Infection and Co-Cultivation of Immature Embryos:

### 2.1 Infection Step:

PHI-A medium of (1) is removed with 1 mL micropipettor, and 1 mL of Agrobacterium suspension is added. The tube is gently inverted to mix. The mixture is incubated for 5 min at room temperature.

### 2.2 Co-Culture Step:

The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for three days. L-Cysteine can be used in the co-cultivation phase. With the standard binary vector, the co-cultivation medium supplied with 100-400 mg/L L-cysteine is critical for recovering stable transgenic events.

### 3. Selection of Putative Transgenic Events:

To each plate of PHI-D medium in a 100×15 mm Petri dish, 10 embryos are transferred, maintaining orientation, and the dishes are sealed with parafilm. The plates are incubated in darkness at 28° C. Actively growing putative events, evinced as pale yellow embryonic tissue, are expected to be visible in six to eight weeks. Embryos that produce no events may be brown and necrotic, and little friable tissue growth is evident. Putative transgenic embryonic tissue is subcultured to fresh PHI-D plates at two-three week intervals, depending on growth rate. The events are recorded.

4. Regeneration of T0 plants:

Embryonic tissue propagated on PHI-D medium is subcultured to PHI-E medium (somatic embryo maturation medium), in 100×25 mm Petri dishes and incubated at 28° C., in darkness, until somatic embryos mature, for about ten to eighteen days. Individual, matured somatic embryos with well-defined scutellum and coleoptile are transferred to PHI-F embryo germination medium and incubated at 28° C. in the light (about 80 μE from cool white or equivalent fluorescent lamps). In seven to ten days, regenerated plants, about 10 cm tall, are potted in horticultural mix and hardened-off using standard horticultural methods.

### Media for Plant Transformation:

1. PHI-A: 4g/L CHU basal salts, 1.0 mL/L 1000× Eriksson's vitamin mix, 0.5 mg/L thiamin HCl, 1.5 mg/L 2,4-D, 0.69 g/L L-proline, 68.5 g/L sucrose, 36 g/L glucose, pH 5.2. Add 100 μM acetosyringone (filter-sterilized).
2. PHI-B: PHI-A without glucose, increase 2,4-D to 2 mg/L, reduce sucrose to 30 g/L and supplemented with 0.85 mg/L silver nitrate (filter-sterilized), 3.0 g/L GELRITE®, 100 μM acetosyringone (filter-sterilized), pH 5.8.
3. PHI-C: PHI-B without GELRITE® and acetosyringonee, reduce 2,4-D to 1.5 mg/L and supplemented with 8.0 g/L agar, 0.5 g/L 2-[N-morpholino]ethane-sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, Cat. No. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; reduce sucrose to 40 g/L; replacing agar with 1.5 g/L GELRITE®; pH 5.6.

Plants can be regenerated from the transgenic callus by first transferring clusters of tissue to N6 medium supplemented with 0.2 mg per liter of 2,4-D. After two weeks the tissue can be transferred to regeneration medium (Fromm et al., Bio/Technology 8:833-839 (1990)).

Transgenic T0 plants can be regenerated and their phenotype determined. T1 seed can be collected.

Furthermore, a recombinant DNA construct containing a validated Arabidopsis gene can be introduced into a maize inbred line either by direct transformation or introgression from a separately transformed line.

Transgenic plants, either inbred or hybrid, can undergo more vigorous field-based experiments to study yield enhancement and/or stability under nitrogen limiting and nitrogen non-limiting conditions.

Subsequent yield analysis can be done to determine whether plants that contain the validated Arabidopsis lead gene have an improvement in yield performance (under nitrogen limiting or non-limiting conditions), when compared to the control (or reference) plants that do not contain the validated Arabidopsis lead gene. Plants containing the validated Arabidopsis lead gene would have less yield loss relative to the control plants, for example, at least 25% less yield loss, under nitrogen limiting conditions, or would have increased yield relative to the control plants under nitrogen non-limiting conditions.

### Example 14A

### Preparation of Expression Vector for Transformation of Maize Lines with Validated Candidate Arabidopsis Gene (At5g43870) Using Agrobacterium

The PH11 sequence with alternative codons (AT-PH11 ac; SEQ ID NO:4) was synthesized and cloned into pUC57 vector to create a plasmid PHP50578. The plasmid PHP50578 was used to create the plasmid PHP51211.

Using INVITROGEN™ GATEWAY® technology, an LR Recombination Reaction was performed with PHP51211 and a destination vector to create a precursor plasmid, PHP51404, which contains the following expression cassettes:

1. Os-Actin promoter::MOPAT::CaMV terminator; cassette expressing the PAT herbicide resistance gene used for selection during the transformation process.

2. LTP2 promoter::DS-RED2::PinII terminator; cassette expressing the DS-RED color marker gene used for seed sorting.

3. Ubiquitin promoter::AT-PH11ac:: SB-GKAF terminator (PCT International Patent Publication Number WO2013019461; incorporated herein by reference); cassette with the AT-PH11 sequence with alternative codons (AT-PH11 ac; SEQ ID NO:4) overexpressing the polypeptide of interest, Arabidopsis AT-PH11 polypeptide.

### Example 14B

### Transformation of Maize Lines with Validated Candidate Arabidopsis Gene (At5g43870) Using Agrobacterium

The AT-PH11 sequence with alternative codons (AT-PH11ac) expression cassette present in the precursor plasmid described in Example 14A can be introduced into a maize inbred line, or a transformable maize line derived from an elite maize inbred line, using Agrobacterium-mediated transformation as described in Examples 12 and 13.

The expression vector (precursor plasmid described in example 14A; (PHP51404) can be electroporated into the LBA4404 Agrobacterium strain containing vector PHP10523 (PCT Publication No. WO/2012/058528) to create a co-integrate vector PHP51603, formed by recombination via COS sites contained on each vector. The cointegrate vector would contain the same three expression cassettes as above (Example 14A) in addition to other genes (TET, TET, TRFA, ORI terminator, CTL, ORI V, VIR C1, VIR C2, VIR G, VIR B) needed for the Agrobacterium strain and the Agrobacterium-mediated transformation. The electroporation protocol in, but not limited to, Example 12 may be used.

### Example 14C

### Preparation of Expression Vector for Transformation of Maize Lines with Maize Homologs

Using the INVITROGEN™ GATEWAY® technology, an LR Recombination Reaction can be performed with an entry clone containing a maize homolog of the Arabidopsis PH11 gene and a destination vector to create a precursor plasmid with the following expression cassettes:

1. Ubiquitin promoter::moPAT::PinII terminator cassette expressing the PAT herbicide resistance gene used for selection during the transformation process.

2. LTP2 promoter::DS-RED2::PinII terminator cassette expressing the DS-RED color marker gene used for seed sorting.

3. Ubiquitin promoter::PH11 homolog from maize (Zm-PH11)::PinII terminator cassette over expressing the gene of interest.

### Example 14D

### Preparation of Expression Vector for Transformation of Maize Lines with Validated Candidate

Arabidopsis Gene (At2g36030) Using Agrobacterium

The NUCPU29 sequence with alternative codons (AT-NUCPU29ac; SEQ ID NO:71) was synthesized and cloned into pUC57 vector to create a plasmid PHP50005. The plasmid PHP50005 was used to create the plasmid PHP50206.

Using INVITROGEN™ GATEWAY® technology, an LR Recombination Reaction was performed with PHP50206 and a destination vector to create a precursor plasmid, PHP50298, which contains the following expression cassettes:

1. Os-Actin promoter::MOPAT::CaMV terminator; cassette expressing the PAT herbicide resistance gene used for selection during the transformation process.

2. LTP2 promoter::DS-RED2::PinII terminator; cassette expressing the DS-RED color marker gene used for seed sorting.

3. Ubiquitin promoter:: AT-NUCPU29ac::Sb-Ubi terminator; cassette with the NUCPU29 sequence with alternative codons (AT-NUCPU29ac; SEQ ID NO:71) overexpressing the polypeptide of interest, Arabidopsis AT-NUCPU29 polypeptide.

### Example 14E

### Transformation of Maize Lines with Validated Candidate Arabidopsis Gene (At2g36030) Using Agrobacterium

The AT-NUCPU29 with alternative codons (AT-NUCPU29ac) expression cassette present in the precursor plasmid described in Example 14A can be introduced into a maize inbred line, or a transformable maize line derived from an elite maize inbred line, using Agrobacterium-mediated transformation as described in Examples 12 and 13.

The expression vector (precursor plasmid described in example 14A; (PHP50298) was electroporated into the LBA4404 Agrobacterium strain containing vector PHP10523 (PCT Publication No. WO/2012/058528) to create a co-integrate vector PHP50351, formed by recombination via COS sites contained on each vector. The cointegrate vector would contain the same three expression cassettes as above (Example 14A) in addition to other genes (TET, TET, TRFA, ORI terminator, CTL, ORI V, VIR C1, VIR C2, VIR G, VIR B) needed for the Agrobacterium strain and the Agrobacterium-mediated transformation. The electroporation protocol in, but not limited to, Example 12 may be used.

### Example 14F

### Preparation of Expression Vector for Transformation of Maize Lines with Maize Homologs

Using the INVITROGEN™ GATEWAY® technology, an LR Recombination Reaction can be performed with an entry clone containing a maize homolog of the Arabidopsis NUCPU29 gene and a destination vector to create a precursor plasmid with the following expression cassettes:

1. Ubiquitin promoter::moPAT::PinII terminator cassette expressing the PAT herbicide resistance gene used for selection during the transformation process.

2. LTP2 promoter::DS-RED2::PinII terminator cassette expressing the DS-RED color marker gene used for seed sorting.

3. Ubiquitin promoter::NUCPU29 homolog from maize::PinII terminator cassette over expressing the gene of interest.

### Example 14G

### Transformation of Maize Lines with Maize Homolog Using Agrobacterium

The expression cassette containing the Zm-PH11 or Zm-NUCPU29, described in Example 14C and Example 14F, can be introduced into a maize inbred line, or a transformable maize line derived from an elite maize inbred line, using Agrobacterium-mediated transformation as described in Examples 12 and 13.

The expression vector (precursor plasmid described in example 14C) can be electroporated into the LBA4404 Agrobacterium strain containing vector PHP10523 to create a co-integrate vector, formed by recombination via COS sites contained on each vector. The cointegrate vector would contain the same three expression cassettes as above (Example 14C) in addition to other genes (TET, TET, TRFA, ORI terminator, CTL, ORI V, VIR C1, VIR C2, VIR G, VIR B) needed for the Agrobacterium strain and the Agrobacterium-mediated transformation. The electroporation protocol in, but not limited to, Example 12 may be used.

### Example 15

### Preparation of the Destination Vector PHP23236 for Transformation into Gaspe Flint Derived Maize Lines

Destination vector PHP23236 (PCT Publication No. WO/2012/058528) can be obtained by transformation of Agrobacterium strain LBA4404 containing PHP10523 (PCT Publication No. WO/2012/058528) with vector PHP23235 (PCT Publication No. WO/2012/058528) and isolation of the resulting co-integration product.

Destination vector PHP23236 can be used in a recombination reaction with an entry clone, as described in Example 16, to create a maize expression vector for transformation of Gaspe Flint derived maize lines.

### Example 16

### Preparation of Expression Constructs for Transformation into Gaspe Flint Derived Maize Lines

Using the INVITROGEN™ GATEWAY® LR Recombination technology, the same entry clone described in Example 5 containing the Arabidopsis PH11 gene, the AT-PH11 sequence with alternative codons (AT-PH11ac; SEQ ID NO:4), Arabidopsis NUCPU29 gene, or the NUCPU29 sequence with alternative codons (AT-NUCPU29ac; SEQ ID NO:71) can be directionally cloned into the GATEWAY® destination vector PHP23236 (PCT Publication No. WO/2012/058528) or into PHP29634 (PCT Publication No. WO/2012/058528) to create an expression vector. Destination vector PHP29634 is similar to destination vector PHP23236, however, destination vector PHP29634 has site-specific recombination sites FRT1 and FRT87 and also encodes the GLYAT4602 selectable marker protein for selection of transformants using glyphosate. The resulting expression vector would contain the cDNA of interest, encoding the Arabidopsis thaliana AT-PH11 or AT-NUCPU29, under control of the UBI promoter and is a T-DNA binary vector for Agrobacterium-mediated transformation into maize as described, but not limited to, the examples described herein.

### Example 17A

### Transformation of Gaspe Flint Derived Maize Lines with Validated Candidate Arabidopsis Genes (At2g36030 and At5g43870)

Maize plants can be transformed to overexpress the Arabidopsis At2g36030 or At5g43870 gene (that may be the sequence with alternative codons), and/or the corresponding homologs from other species, in order to examine the resulting phenotype. Expression constructs such as the one described in Example 16 may be used.

Recipient Plants

Recipient plant cells can be from a uniform maize line having a short life cycle (“fast cycling”), a reduced size, and high transformation potential. Typical of these plant cells for maize are plant cells from any of the publicly available Gaspe Flint (GF) line varieties. One possible candidate plant line variety is the F1 hybrid of GF×QTM (Quick Turnaround Maize, a publicly available form of Gaspe Flint selected for growth under greenhouse conditions) disclosed in Tomes et al. (U.S. application Ser. No. 10/367,416 filed Feb. 13, 2003; U.S. Patent Publication No. 2003/0221212 A1 published Nov. 27, 2003). Transgenic plants obtained from this line are of such a reduced size that they can be grown in four inch pots (¼ the space needed for a normal sized maize plant) and mature in less than 2.5 months. (Traditionally 3.5 months is required to obtain transgenic T0 seed once the transgenic plants are acclimated to the greenhouse.) Another suitable line includes but is not limited to a double haploid line of GS3 (a highly transformable line) X Gaspe Flint. Yet another suitable line is a transformable elite maize inbred line carrying a transgene which causes early flowering, reduced stature, or both.

Transformation Protocol

Any suitable method may be used to introduce the transgenes into the maize cells, including but not limited to, inoculation type procedures using Agrobacterium based vectors (see, for example, Examples 12 and 13). Transformation may be performed on immature embryos of the recipient (target) plant.

Precision Growth and Plant Tracking

The event population of transgenic (T0) plants resulting from the transformed maize embryos is grown in a controlled greenhouse environment using a modified randomized block design to reduce or eliminate environmental error. A randomized block design is a plant layout in which the experimental plants are divided into groups (e.g., thirty plants per group), referred to as blocks, and each plant is randomly assigned a location within the block.

For a group of thirty plants, twenty-four transformed, experimental plants and six control plants (plants with a set phenotype) (collectively, a “replicate group”) are placed in pots which are arranged in an array (a.k.a. a replicate group or block) on a table located inside a greenhouse. Each plant, control or experimental, is randomly assigned to a location within the block which is mapped to a unique, physical greenhouse location as well as to the replicate group. Multiple replicate groups of thirty plants each may be grown in the same greenhouse in a single experiment. The layout (arrangement) of the replicate groups should be determined to minimize space requirements as well as environmental effects within the greenhouse. Such a layout may be referred to as a compressed greenhouse layout.

An alternative to the addition of a specific control group is to identify those transgenic plants that do not express the gene of interest. A variety of techniques such as RT-PCR can be applied to quantitatively assess the expression level of the introduced gene. T0 plants that do not express the transgene can be compared to those which do.

Each plant in the event population is identified and tracked throughout the evaluation process, and the data gathered from that plant is automatically associated with that plant so that the gathered data can be associated with the transgene carried by the plant. For example, each plant container can have a machine readable label (such as a Universal Product Code (UPC) bar code) which includes information about the plant identity, which in turn is correlated to a greenhouse location so that data obtained from the plant can be automatically associated with that plant.

Alternatively any efficient, machine readable, plant identification system can be used, such as two-dimensional matrix codes or even radio frequency identification tags (RFID) in which the data is received and interpreted by a radio frequency receiver/processor. See U.S. application Ser. No. 10/324,288 filed Dec. 19, 2002 (U.S. Patent Publication No. 2004/0122592 A1 published Jun. 24, 2004), incorporated herein by reference.

Phenotypic Analysis Using Three-Dimensional Imaging

Each greenhouse plant in the T0 event population, including any control plants, is analyzed for agronomic characteristics of interest, and the agronomic data for each plant is recorded or stored in a manner so that it is associated with the identifying data (see above) for that plant. Confirmation of a phenotype (gene effect) can be accomplished in the T1 generation with a similar experimental design to that described above.

The T0 plants are analyzed at the phenotypic level using quantitative, non-destructive imaging technology throughout the plant's entire greenhouse life cycle to assess the traits of interest. A digital imaging analyzer may be used for automatic multi-dimensional analyzing of total plants. The imaging may be done inside the greenhouse. Two camera systems, located at the top and side, and an apparatus to rotate the plant, are used to view and image plants from all sides. Images are acquired from the top, front and side of each plant. All three images together provide sufficient information to evaluate, for example, the biomass, size, and morphology of each plant.

Due to the change in size of the plants from the time the first leaf appears from the soil to the time the plants are at the end of their development, the early stages of plant development are best documented with a higher magnification from the top. This imaging may be accomplished by using a motorized zoom lens system that is fully controlled by the imaging software.

In a single imaging analysis operation, the following events occur: (1) the plant is conveyed inside the analyzer area, rotated 360 degrees so its machine readable label can be read, and left at rest until its leaves stop moving; (2) the side image is taken and entered into a database; (3) the plant is rotated 90 degrees and again left at rest until its leaves stop moving, and (4) the plant is transported out of the analyzer.

Plants are allowed at least six hours of darkness per twenty four hour period in order to have a normal day/night cycle.

Imaging Instrumentation

Any suitable imaging instrumentation may be used, including but not limited to light spectrum digital imaging instrumentation commercially available from LemnaTec GmbH of Wurselen, Germany. The images are taken and analyzed with a LemnaTec Scanalyzer HTS LT-0001-2 having a ½″ IT Progressive Scan IEE CCD imaging device. The imaging cameras may be equipped with a motor zoom, motor aperture, and motor focus. All camera settings may be made using LemnaTec software. For example, the instrumental variance of the imaging analyzer is less than about 5% for major components and less than about 10% for minor components.

Software

The imaging analysis system comprises a LemnaTec HTS Bonit software program for color and architecture analysis and a server database for storing data from about 500,000 analyses, including the analysis dates. The original images and the analyzed images are stored together to allow the user to do as much reanalyzing as desired. The database can be connected to the imaging hardware for automatic data collection and storage. A variety of commercially available software systems (e.g., Matlab, others) can be used for quantitative interpretation of the imaging data, and any of these software systems can be applied to the image data set.

Conveyor System

A conveyor system with a plant rotating device may be used to transport the plants to the imaging area and rotate them during imaging. For example, up to four plants, each with a maximum height of 1.5 m, are loaded onto cars that travel over the circulating conveyor system and through the imaging measurement area. In this case the total footprint of the unit (imaging analyzer and conveyor loop) is about 5 m×5 m.

The conveyor system can be enlarged to accommodate more plants at a time. The plants are transported along the conveyor loop to the imaging area and are analyzed for up to 50 seconds per plant. Three views of the plant are taken. The conveyor system, as well as the imaging equipment, should be capable of being used in greenhouse environmental conditions.

Illumination

Any suitable mode of illumination may be used for the image acquisition. For example, a top light above a black background can be used. Alternatively, a combination of top- and backlight using a white background can be used. The illuminated area should be housed to ensure constant illumination conditions. The housing should be longer than the measurement area so that constant light conditions prevail without requiring the opening and closing or doors. Alternatively, the illumination can be varied to cause excitation of either transgene (e.g., green fluorescent protein (GFP), red fluorescent protein (RFP)) or endogenous (e.g. Chlorophyll) fluorophores).

Biomass Estimation Based on Three-Dimensional Imaging

For best estimation of biomass the plant images should be taken from at least three axes, for example, the top and two side (sides 1 and 2) views. These images are then analyzed to separate the plant from the background, pot and pollen control bag (if applicable). The volume of the plant can be estimated by the calculation:

Volume(voxels)=\sqrt{TopArea(pixels)}×\sqrt{Side1Area(pixels)}×\sqrt{Side2Area(pixels)}

In the equation above the units of volume and area are “arbitrary units”. Arbitrary units are entirely sufficient to detect gene effects on plant size and growth in this system because what is desired is to detect differences (both positive-larger and negative-smaller) from the experimental mean, or control mean. The arbitrary units of size (e.g. area) may be trivially converted to physical measurements by the addition of a physical reference to the imaging process. For instance, a physical reference of known area can be included in both top and side imaging processes. Based on the area of these physical references a conversion factor can be determined to allow conversion from pixels to a unit of area such as square centimeters (cm²). The physical reference may or may not be an independent sample. For instance, the pot, with a known diameter and height, could serve as an adequate physical reference.

Color Classification

The imaging technology may also be used to determine plant color and to assign plant colors to various color classes. The assignment of image colors to color classes is an inherent feature of the LemnaTec software. With other image analysis software systems color classification may be determined by a variety of computational approaches.

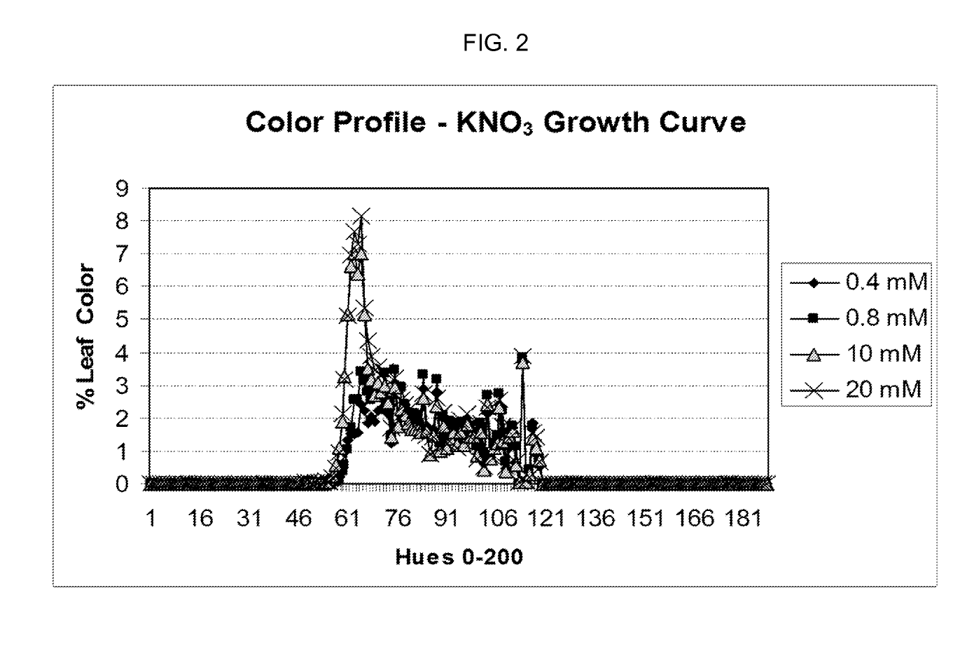

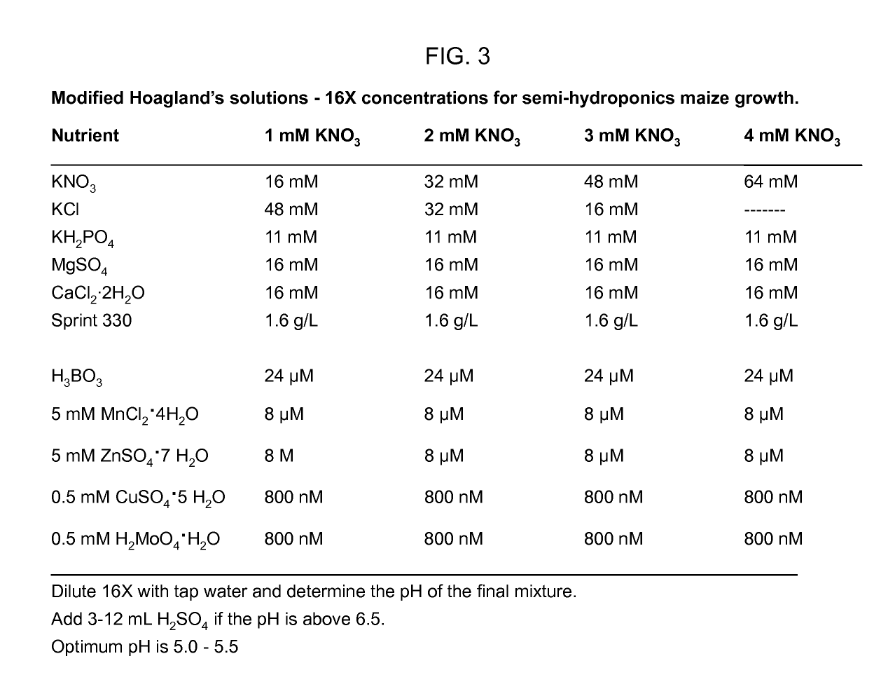

For the determination of plant size and growth parameters, a useful classification scheme is to define a simple color scheme including two or three shades of green (for example, hues 50-66, see FIG. 2) and, in addition, a color class for chlorosis, necrosis and bleaching, should these conditions occur. A background color class which includes non plant colors in the image (for example pot and soil colors) is also used and these pixels are specifically excluded from the determination of size. The plants are analyzed under controlled constant illumination so that any change within one plant over time, or between plants or different batches of plants (e.g. seasonal differences) can be quantified.

In addition to its usefulness in determining plant size growth, color classification can be used to assess other yield component traits. For these other yield component traits additional color classification schemes may be used. For instance, the trait known as “staygreen”, which has been associated with improvements in yield, may be assessed by a color classification that separates shades of green from shades of yellow and brown (which are indicative of senescing tissues). By applying this color classification to images taken toward the end of the T0 or T1 plants' life cycle, plants that have increased amounts of green colors relative to yellow and brown colors (expressed, for instance, as Green/Yellow Ratio) may be identified. Plants with a significant difference in this Green/Yellow ratio can be identified as carrying transgenes which impact this important agronomic trait.

The skilled plant biologist will recognize that other plant colors arise which can indicate plant health or stress response (for instance anthocyanins), and that other color classification schemes can provide further measures of gene action in traits related to these responses.

Plant Architecture Analysis

Transgenes which modify plant architecture parameters may also be identified using the present disclosure, including such parameters as maximum height and width, internodal distances, angle between leaves and stem, number of leaves starting at nodes, and leaf length. The LemnaTec system software may be used to determine plant architecture as follows. The plant is reduced to its main geometric architecture in a first imaging step and then, based on this image, parameterized identification of the different architecture parameters can be performed. Transgenes that modify any of these architecture parameters either singly or in combination can be identified by applying the statistical approaches previously described.

Pollen Shed Date

Pollen shed date is an important parameter to be analyzed in a transformed plant, and may be determined by the first appearance on the plant of an active male flower. To find the male flower object, the upper end of the stem is classified by color to detect yellow or violet anthers. This color classification analysis is then used to define an active flower, which in turn can be used to calculate pollen shed date.

Alternatively, pollen shed date and other easily visually detected plant attributes (e.g., pollination date, first silk date) can be recorded by the personnel responsible for performing plant care. To maximize data integrity and process efficiency, this data is tracked by utilizing the same barcodes utilized by the LemnaTec light spectrum digital analyzing device. A computer with a barcode reader, a palm device, or a notebook PC may be used for ease of data capture recording time of observation, plant identifier, and the operator who captured the data.

Orientation of the Plants

Mature maize plants grown at densities approximating commercial planting often have a planar architecture. That is, the plant has a clearly discernable broad side, and a narrow side. The image of the plant from the broadside is determined. To each plant a well defined basic orientation is assigned to obtain the maximum difference between the broadside and edgewise images. The top image is used to determine the main axis of the plant, and an additional rotating device is used to turn the plant to the appropriate orientation prior to starting the main image acquisition.

### Example 17B

### Transformation of Gaspe Flint Derived Maize Lines with Maize Homologs

Using the INVITROGEN™ GATEWAY® LR Recombination technology, entry clones may be created for the maize homologs given in Table 1, and can be directionally cloned into the GATEWAY® destination vector PHP23236 (PCT Publication No. WO/2012/058528) to create a corresponding expression vector. Each expression vector would contain the cDNA of interest under control of the UBI promoter and would be a T-DNA binary for Agrobacterium-mediated transformation into maize as described, but not limited to, the examples described herein.

### Example 18

### Screening of Maize Lines Under Nitrogen Limiting Conditions

Gaspe Flint Derived Maize Lines

Transgenic plants can contain two or three doses of Gaspe Flint-3 with one dose of GS3 (GS3/(Gaspe-3)2× or GS3/(Gaspe-3)3×) and segregate 1:1 for a dominant transgene. Transgenic plants can be planted in 100% TURFACE® medium, a commercial potting medium, and can be watered four times each day with 1 mM KNO₃ growth medium and with 2 mM KNO₃, or higher, growth medium (see FIG. 12). Control plants grown in 1 mM KNO₃ medium would be less green, produce less biomass, and have a smaller ear at anthesis. Gaspe-derived lines would be grown to the flowering stage.

Statistics would be used to decide if differences seen between treatments are really different. One method is that which places letters after the values. Those values in the same column that have the same letter (not group of letters) following them are not significantly different. Using this method, if there are no letters following the values in a column, then there are no significant differences between any of the values in that column or, in other words, all the values in that column are equal.

Expression of a transgene would result in plants with improved plant growth in 1 mM KNO₃ when compared to a transgenic null. Thus biomass and greenness (as described in Example 2 and 17A) would be monitored during growth and compared to a transgenic null. Improvements in growth, greenness, and ear size at anthesis would be indications of increased nitrogen tolerance.

Seedling Assay

Transgenic maize plants can also be evaluated using a seedling assay that assesses plant performance under nitrogen limiting conditions. In an 18 day seedling assay, for example, transgenic plants are planted in TURFACE® medium, a commercial potting medium, and then watered four times each day with a solution containing the following nutrients: 1 mM CaCl₂, 2 mM MgSO₄, 0.5 mM KH₂PO₄, 83 ppm Sprint330, 3 mM KCl, 1 mM KNO₃, 1 μM ZnSO₄, 1 μM MnCl₂, 3 μM H₃BO₄, 0.1 μM CuSO₄, and 0.1 μM NaMoO₄. Plants are harvested 18 days after planting, and a number of traits are assessed, including but not limited to: SPAD (greenness), stem diameter, root dry weight, shoot dry weight, total dry weight, mg Nitrogen per grams of dry weight (mg N/g dwt), and plant N concentration. Means are compared to null mean parameters using a Student's t test with a minimum (P<t) of 0.1.

### Example 19

### Nitrogen Utilization Efficiency Seedling Assay

Experiments can be performed using seed of transgenic events. Seed of transgenic events can be separated into Transgenic (Treatment 1; contain construct) and Null (Treatment 2) seed using a seed color marker. In a second experiment, seed of transgenic events can be separated into Transgenic (Treatment 1; contain construct) and Null (Treatment 2) seed using a seed color marker.

All seeds are planted in 4 inch, square pots containing TURFACE® medium on 8 inch, staggered centers and watered four times each day with a solution containing the following nutrients:

| | | | |
|---|---|---|---|
| 1 mM CaCl₂ | 2 mM MgSO₄ | 0.5 mM KH₂PO₄ | 83 ppm Sprint330 |
| 3 mM KCl | 1 mM KNO₃ | 1 μM ZnSO₄ | 1 μM MnCl₂ |
| 3 μM H₃BO₄ | 1 μM MnCl₂ | 0.1 μM CuSO₄ | 0.1 μM NaMoO₄ |

After emergence the plants are thinned to one seed per pot. At harvest, plants are removed from the pots, and the TURFACE® medium is washed from the roots. The roots are separated from the shoot, placed in a paper bag, and dried at 70° C. for 70 hr. The dried plant parts (roots and shoots) are weighed and placed in a 50 ml conical tube with approximately 20 5/32 inch steel balls and then ground by shaking in a paint shaker.

The Nitrogen/Protein Analyzer from Thermo Electron Corporation (model FlashEA 1112 N) uses approximately 30 mg of the ground tissue. A sample is dropped from the Autosampler into the crucible inside the oxidation reactor chamber. At 900° C. and pure oxygen, the sample is oxidized by a strong exothermic reaction creating a gas mixture of N₂, CO₂, H₂O, and SO₂. After the combustion is complete, the carrier gas helium is turned on and the gas mixture flows into the reduction reaction chamber. At 680° C., the gas mixture flows across the reduction copper where nitrogen oxides possibly formed are converted into elemental nitrogen and the oxygen excess is retained. From the reduction reactor, the gas mixture flows across a series of two absorption filters. The first filter contains soda lime and retains carbon and sulfur dioxides. The second filter contains molecular sieves and granular silica gel to hold back water. Nitrogen is then eluted in the chromatographic column and conveyed to the thermal conductivity detector that generates an electrical signal, which, properly processed by the Eager 300 software, provides the nitrogen-protein percentage.

Using these data, the following parameters can be measured and means of Transgenic parameters were compared to means of Null parameters using a Student's t test:

| | |
|---|---|
| Total Plant Biomass | (total dry weight) |
| Root Biomass | (root dwt) |
| Shoot Biomass | (shoot dwt) |
| Root/Shoot Ratio | (root shoot dwt ratio) |
| Plant N concentration | (mg N/g dwt) |
| Total Plant N | (total N (mg)) |
| Total % Vegetative N | (total veg nitrogen) |
| Shoot Total N | (mg) |

Variance is calculated within each block using an Analysis of Variance (ANOVA) calculation and a completely random design (CRD) model. An overall treatment effect for each block can be calculated using an F statistic by dividing overall block treatment mean square by the overall block error mean square. The probability of a greater Student's t test can be calculated for each transgenic mean compared to the appropriate null.

A minimum (P<t) of 0.1 can be used to define variables that show a significant difference.

### Example 20A

### Yield Analysis of Maize Lines with the Arabidopsis PH11 or NUCPU29 or Maize Homologs

Transgenic plants, either inbred or hybrid, can undergo more vigorous field-based experiments to study yield enhancement and/or stability under nitrogen limiting and non-limiting conditions.

Yield analysis can be done to determine whether plants that contain the validated Arabidopsis PH11 gene, Arabidopsis NUCPU29, or a maize homolog of PH11 or NUCPU29, have an improvement in yield performance (under nitrogen limiting or non-limiting conditions), when compared to the control (or reference) plants that do not contain the validated Arabidopsis PH11 or NUCPU29 gene or a maize homolog of PH11 or NUCPU29. Specifically, nitrogen limiting conditions can be imposed during the flowering and/or grain fill period for plants that contain either the validated Arabidopsis lead gene or a maize homolog of PH11 or NUCPU29 and the control plants. Reduction in yield can be measured for both. Plants containing the validated Arabidopsis lead gene or a maize homolog of PH11 or NUCPU29 would have less yield loss relative to the control plants, for example, at least 25%, at least 20%, at least 15%, at least 10% or at least 5% less yield loss, under nitrogen limiting conditions, or would have increased yield relative to the control plants under nitrogen non-limiting conditions, for example, at least 25% for example, at least 25%, at least 20%, at least 15%, at least 10% or at least 5% increased yield.

### Example 20B

### Yield Analysis of Maize Lines Transformed with PHP51603 Expressing the Arabidopsis Lead Gene At5g43870

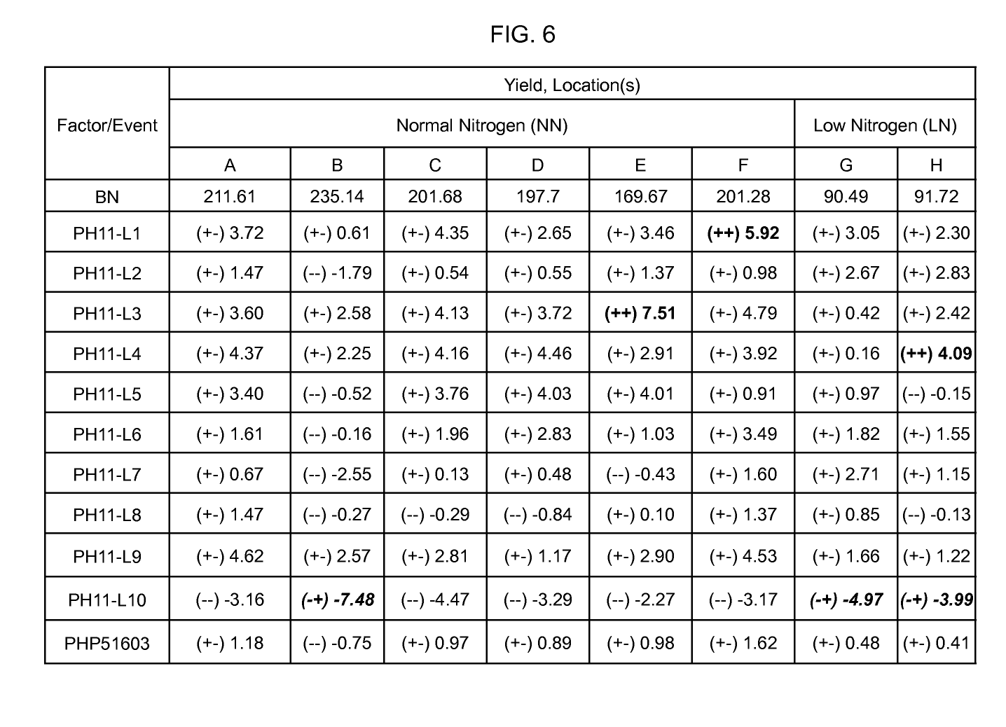

Ten transgenic events were field tested at two low-N locations (“LN”; average yield 91 bu/ac) and at six locations where soil N levels were considered normal for maize production (“NN”; average yield 203 bu/ac). Locations with normal nitrogen (NN) conditions are shown as A-F in FIG. 6, locations with low nitrogen (LN; stress conditions) are shown as G and H locations in FIG. 6.

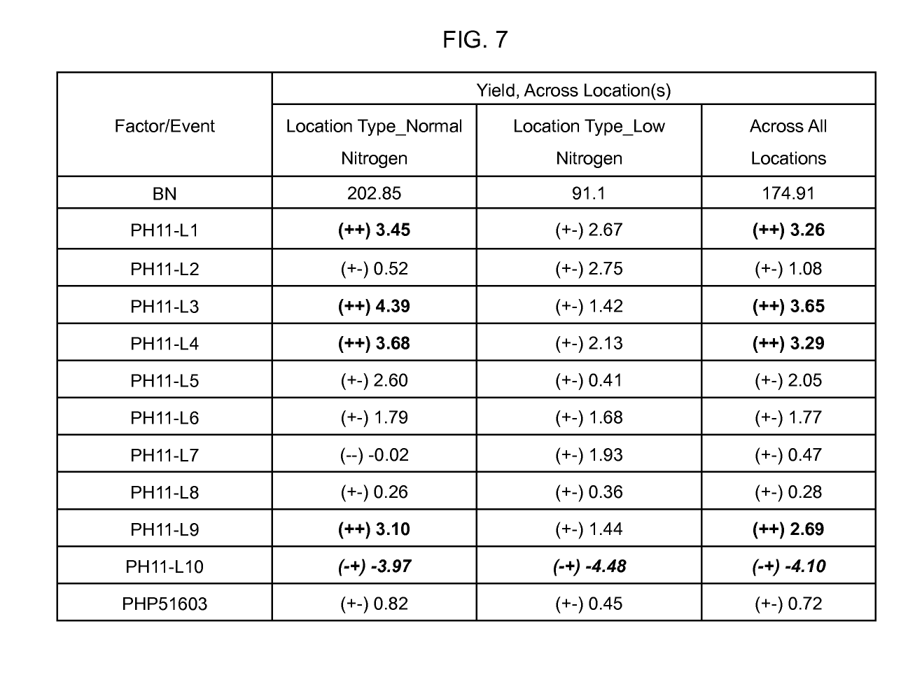

FIG. 7 shows the across location yield analysis for all locations; across all normal nitrogen locations (locations A-F); and across all low nitrogen conditions (locations G and H)

Yield data were collected in all locations, with 3-4 replicates per location.

Yield data (bushel/acre; bu/ac) for the 10 transgenic events are shown in FIG. 6 together with the bulk null control (BN). Yield analysis was by ASREML (VSN International Ltd), and the values are BLUPs (Best Linear Unbiased Prediction) (Cullis, B. R. et al. (1998) Biometrics 54:1-18; Gilmour, A. R. et al. (2009). ASRemI User Guide 3.0; Gilmour, A. R., et al. (1995) Biometrics 51:1440-50).

As shown in FIG. 6, the effect of the transgene on yield was positive and significant for at least one event in one of the stress (low nitrogen) locations, and in 2 normal nitrogen locations. The effect of the transgene was positive and significant for at least 4 events in 2 of the across-location analyses (all locations, and NN locations).

In addition to the values for the individual events described in FIG. 6 and FIG. 7, the row labeled with the plasmid name, PHP51603, provides the construct-level analysis.

### Example 20C

### Yield Analysis of Maize Lines Transformed with PHP50351 Expressing the Arabidopsis Lead Gene At2g36030 (AT-NUCPU29)

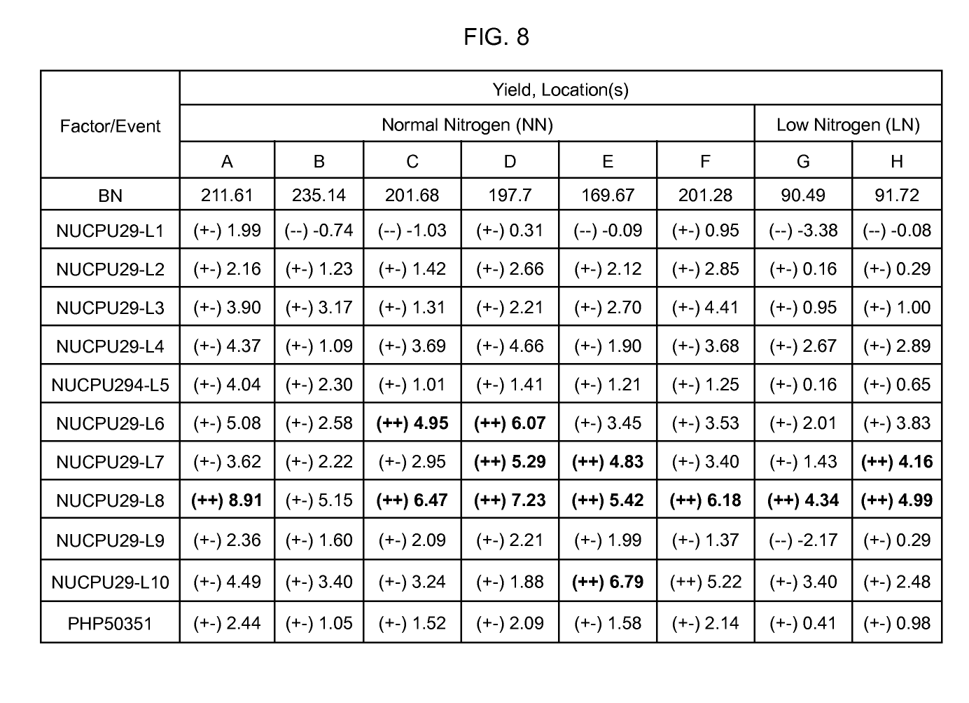

Ten transgenic events were field tested at two low-N locations (“LN”; average yield 91 bu/ac) and at six locations where soil N levels were considered normal for maize production (“NN”; average yield 203 bu/ac). Locations with normal nitrogen (NN) conditions are shown as A-F in FIG. 8, locations with low nitrogen (LN; stress conditions) are shown as G and H locations in FIG. 8.

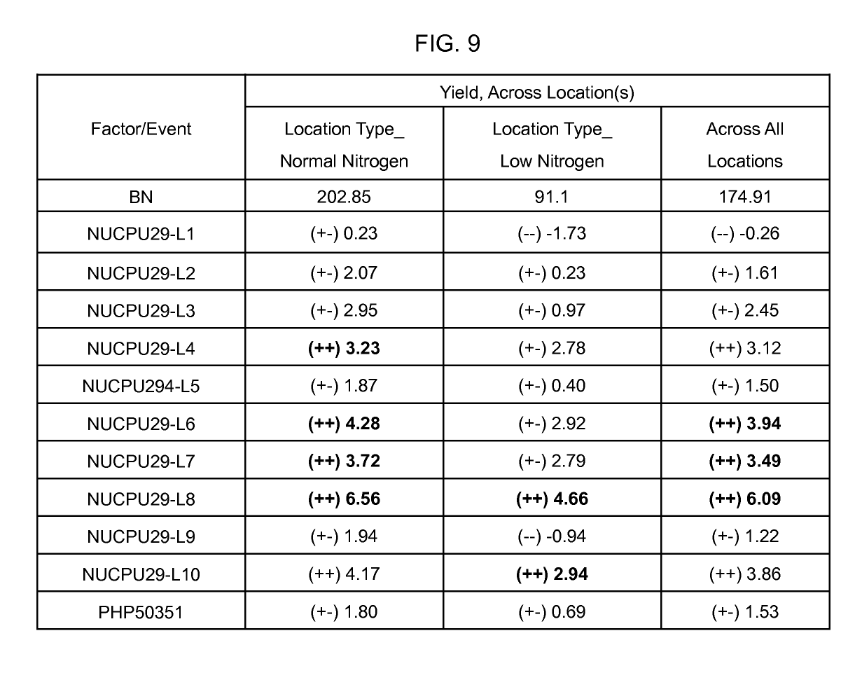

FIG. 9 shows the across location yield analysis for all locations; across all normal nitrogen locations (locations A-F); and across all low nitrogen conditions (locations G and H)

Yield data were collected in all locations, with 3-4 replicates per location. The values are BLUPs for the difference from the null in bushel/acre (bu/ac). The BN value is the yield in bu/ac for the null.

Yield data (bushel/acre; bu/ac) for the 10 transgenic events are shown in FIG. 8 together with the bulk null control (BN). Yield analysis was by ASREML (VSN International Ltd), and the values are BLUPs (Best Linear Unbiased Prediction) (Cullis, B. R et al. (1998) Biometrics 54: 1-18, Gilmour, A. R. et al. (2009). ASRemI User Guide 3.0, Gilmour, A. R., et al. (1995) Biometrics 51: 1440-50).

As shown in FIG. 8, the effect of the transgene on yield was positive and significant for at least one event in both the stress (low nitrogen) locations, and in 4 normal nitrogen locations. The effect of the transgene was positive and significant for at least 2 events in the all the across-location analyses (all locations, LN locations, NN locations).

In addition to the values for the individual events described in FIG. 8 and FIG. 9, the row labeled with the plasmid name, PHP50351, provides the construct-level analysis.

### Example 21

### Transformation and Evaluation of Soybean with Homologs of PH11 or NUCPU29 Polypeptides

Homologs of the validated Arabidopsis AT-PH11 or AT-NUCPU29 leads identified as described in previous Examples, can be assessed for their ability to enhance tolerance to nitrogen limiting conditions in soybean. Vector construction, plant transformation and phenotypic analysis will be similar to that in previously described Examples.

### Example 22

### Transformation of Arabidopsis with Homologs of PH11 or NUCPU29 Polypeptides

Homologs of AT-PH11 polypeptide identified as described in previous examples, can be transformed into Arabidopsis under control of the 35S promoter, and any other tissue-specific promoters, and assayed for leaf area and green color bin accumulation when grown on low nitrogen medium. Vector construction and plant transformation can be as described in the examples herein. Assay conditions, data capture and data analysis can be similar to that in previously described Examples.

### Example 23

### Transformation and Evaluation of Maize with Homologs of Validated Lead Genes

Homologs of AT-PH11 or AT-NUCPU29 polypeptides identified as described in previous examples, can be assessed for their ability to enhance tolerance to nitrogen limiting conditions in maize. Vector construction, plant transformation and phenotypic analysis can be similar to that in previously described Examples.

### Example 24A

### Root Architecture Assay

To test transgenic plants for alteration in root architecture, the root architecture assay can be done as described in this Example.

Seeds are sterilized using 50% household bleach 0.01% triton X-100 solution and on petri plates containing the following medium: 0.5× N-Free Hoagland's, 60 mM KNO₃, 0.1% sucrose, 1 mM MES and 1% PHYTAGEL™ at a density of 4 seeds/plate. Typically 10 plates are placed in a rack. Plates are kept for three days at 4° C. to stratify seeds and then held vertically for 14 days at 22° C. light and 20° C. dark. Photoperiod is 16 h light; 8 h dark, average light intensity is ˜180 μmol/m²/s. Racks (typically holding 10 plates each) are rotated every alternate day within each shelf. At day 14, plates are evaluated for seedling status, whole plate scan are taken, and analyzed for root area.

These seedlings grown on vertical plates are analyzed for root growth with the WINRHIZO® software (Regent Instruments Inc), an image analysis system specifically designed for root measurement. WINRHIZO® software uses the contrast in pixels to distinguish the light root from the darker background. To identify the maximum amount of roots without picking up background, the pixel classification is kept at 150-170 and the filter feature is used to remove objects that have a length/width ratio less than 10.0. The area on the plates analyzed is from the edge of the plant's leaves to about 1 cm from the bottom of the plate. The exact same WINRHIZO® software settings and area of analysis is used to analyze all plates within a batch. The total root length score given by WINRHIZO® software for a plate is divided by the number of plants that have germinated and have grown halfway down the plate. Eight plates for every line are grown and their scores are averaged. This average is then compared to the average of eight plates containing wild type seeds that have been grown at the same time.

Thirty seedlings from a transgenic line are compared to same number from a control line and a probability value is generated. A transgenic line with a probability value (p-value) equal to and or more than E-03 is considered is considered a validated lead.

### Example 24B

### Root Architecture Assay of Transgenic Arabidopsis Seeds with AT-NUCPU29 Polypeptides

T1 seeds from transgenic Arabidopsis line with At-NUCPU29 protein, containing the 35S promoter::At2g36030 expression construct pBC-Yellow-At2g36030, generated as described in Example 5, were tested for enhancement of root architecture as described in Example 24A.

Non-transformed Columbia seed discarded from fluorescent seed sorting served as a control. T1 and control seeds were subjected to the Root Architecture Assay following the procedure described in Example 24A.

Eight plates having 32 seedlings were analyzed statistically and a trend was detected between the number of plants growing on a plate and their average WINRHIZO® score. WINRHIZO® scores were normalized for this trend and the root score corresponding to the construct was divided by the wild type root score.

The p-value for AT-NUCPU29 in the Root Architecture assay was found to be E-05.

What is claimed is:

1. A plant comprising in its genome a recombinant DNA construct comprising a polynucleotide operably linked to at least one heterologous regulatory element, wherein said polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70, and wherein said plant exhibits at least one trait selected from the group consisting of: increased nitrogen stress tolerance, increased yield, increased biomass, and altered root architecture, when compared to a control plant not comprising said recombinant DNA construct.

2. The plant of claim 1, wherein the altered root architecture is an increase in root biomass or in average root length, or both.

3. The plant of claim 1, wherein the polynucleotide is operably linked to a heterologous promoter that is preferably expressed in at least one tissue selected from the group consisting of: root, shoot and vasculature.

4. The plant of claim 1, wherein said plant exhibits an increase in yield, biomass, or both, when compared, under nitrogen limiting conditions, to said control plant.

5. The plant of any one of claim 1, wherein said plant is selected from the group consisting of: maize, soybean, sunflower, sorghum, canola, wheat, alfalfa, cotton, rice, barley, millet, sugar cane and switchgrass.

6. Seed of the plant of claim 1, wherein said seed comprises in its genome a recombinant DNA construct comprising a polynucleotide operably linked to at least one heterologous regulatory element, wherein said polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70, and wherein a plant produced from said seed exhibits at least one trait selected from the group consisting of: increased nitrogen stress tolerance, increased yield, increased biomass, and altered root architecture, when compared to a control plant not comprising said recombinant DNA construct.

7. A method of increasing nitrogen stress tolerance in a plant, comprising:
(a) introducing into a regenerable plant cell a recombinant DNA construct comprising a polynucleotide operably linked to at least one heterologous regulatory element, wherein the polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70;
(b) regenerating a transgenic plant from the regenerable plant cell after step (a), wherein the transgenic plant comprises in its genome the recombinant DNA construct; and
(c) obtaining a progeny plant derived from the transgenic plant of step (b), wherein said progeny plant comprises in its genome the recombinant DNA construct and exhibits increased nitrogen stress tolerance when compared to a control plant not comprising the recombinant DNA construct.

8. The method of claim 8 wherein the polynucleotide in step (a) is operably linked to a heterologous promoter and wherein the promoter is expressed in at least one tissue selected from the group consisting of: root, shoot and vasculature.

9. A method of selecting for a plant that exhibits at least one trait selected from the group consisting of: increased nitrogen stress tolerance, increased yield, increased biomass, and altered root architecture, the method comprising:
(a) obtaining a transgenic plant, wherein the transgenic plant comprises in its genome a recombinant DNA construct comprising a polynucleotide operably linked to at least one heterologous regulatory element, wherein said polynucleotide encodes a polypeptide having an amino acid sequence of at least 80% sequence identity, based on the Clustal V method of alignment, when compared to SEQ ID NO:3, 7, 9, 11, 13, 15, 17, 19, 21, 23, 25, 27, 29, 31, 33-62 or 70;
(b) growing the transgenic plant of part (a) under conditions wherein the polynucleotide is expressed; and
(c) selecting the transgenic plant of part (b) that exhibits at least one trait selected from the group consisting of: increased nitrogen stress tolerance, increased yield, increased biomass, and altered root architecture, when compared to a control plant not comprising the recombinant DNA construct.

10. The method of claim 9, wherein said selecting step (c) comprises determining whether the transgenic plant of (b) exhibits an alteration of yield, biomass or both when compared, under nitrogen limiting conditions, to a control plant not comprising the recombinant DNA construct.

11. The method of claim 9, wherein said altered root architecture is an increase in root biomass or in average root length, or both.

12. The method of any one of claim 9, wherein said plant is selected from the group consisting of: maize, soybean, sunflower, sorghum, canola, wheat, alfalfa, cotton, rice, barley, millet, sugar cane and switchgrass.

13. A recombinant DNA construct comprising a polynucleotide operably linked to at least one heterologous regulatory element, wherein said polynucleotide comprising:
(a) a nucleotide sequence encoding a PH11 or NUCPU29 polypeptide, wherein the polypeptide has nitrogen stress tolerance activity, and wherein the polypeptide has an amino acid sequence of at least 80% sequence identity, based on the Clustal V method of alignment with pairwise alignment default parameters of KTUPLE=1, GAP PENALTY=3, WINDOW=5 and DIAGONALS SAVED=5, when compared to SEQ ID NO:3, 7, 9, 11, 13, 15, 17, 19, 21, 23, 25, 27, 29, 31, 33-62 or 70; or
(b) the full complement of the nucleotide sequence of (a).

14. The polynucleotide of claim 13, wherein the amino acid sequence of the polypeptide comprises SEQ ID NO:3, 7, 9, 11, 13, 15, 17, 19, 21, 23, 25, 27, 29, 31, 33-62 or 70.

15. The polynucleotide of claim 13 wherein the nucleotide sequence comprises SEQ ID NO:2, 4, 6, 8, 10, 12, 14, 16, 18, 20, 22, 24, 26, 28, 30, 32, 69 or 71.

16. A method of producing a plant that exhibits at least one trait selected from the group consisting of: increased nitrogen stress tolerance, increased yield, increased biomass, and altered root architecture, wherein the method comprises growing a plant from the seed of claim 6, wherein the plant exhibits at least one trait selected from the group consisting of: increased nitrogen stress tolerance, increased yield, increased biomass, and altered root architecture, when compared to a control plant not comprising the recombinant DNA construct.

17. A method of producing a seed, the method comprising the following:
(a) crossing a first plant with a second plant, wherein at least one of the first plant and the second plant is the plant of claim 1; and
(b) selecting a seed of the crossing of step (a), wherein the seed comprises the recombinant DNA construct of claim 1.

18. The method of claim 17, wherein a plant grown from the seed of part (b) exhibits at least one trait selected from the group consisting of: increased nitrogen stress tolerance, increased yield, increased biomass, and altered root architecture, when compared to a control plant not comprising the recombinant DNA construct.

19. A method of producing oil or a seed by-product, or both, from a seed, the method comprising extracting oil or a seed by-product, or both, from the seed of claim 6.

20. The method of claim 19, wherein the oil or the seed by-product, or both, comprises the recombinant DNA construct.


SEQUENCE LISTING (73 sequences)

SEQ ID NO: 1 - DNA, 50 bp, Artificial Sequence
  polylinker sequence
gatcactagtggcgcgcctaggagatctcgagtagggataacagggtaat

SEQ ID NO: 2 - DNA, 1362 bp, Arabidopsis thaliana
atggatcaaaaaagtaaaacgattggagaacaatggtggaggtcaaatccaacgtttaaaccgcctgaaacgccattagattctatggagtttttgtcacgtacttggagtgcttccgctactgaagtttcaagagctgtcgtcgcgtctccaccgacttctcaaccgccgcaaatgcgtttctcggagatccaaaacggttcttctgacgtcactttggtgccggaagatgaagaaaacggcatcgttcttggaaatactttttcttttgcttcttcagaaacttctttaatggtcatggaacgtatcatggctcagtcaccggagatttcatcgccacgaacatcagggagactttctcatagctcattcaccgacagtcctccgatctctccctccgacatcgacgactttaagcaattctaccgtgtgagcccttcctttaacggccacatacgtggttcatcagccattcccggcaccgccggagggtctaaaactgttggtcgttggctaaaggaccggcgagagaagaagagagaagagacgcgtgcacaaaatgcacagcttcacgcggctgtatctgtagctggagtggctgccgcggtggctgctatcgctgcagccactgcctctcagtcgagttctggaactgacgagcaagtggccaaaaatgactccgcggtggcttctgccgcgactttggtggcggcgaagtgtgtggaagctgcagagattatgggagctgatcgtgagcacttggcctccgttgttagttctgcggttaacgttcgttctgccggagatatcatgactttgaccgccgctgctgccacagctttgagaggagctgcacaattaaaggcaagagcattgaaggaggtatggaacattgcggctgtgattcctgtagataagggtacaccaaaaggcggtggtggtggttatagaggtggcgagttagcccctgtagataattttcttgggatttgcagtaaagaattgctagctaaaggttgcgaattgcttaaacgcacccgcaaaggtgatcttcattggaaagttgtttcgatctacattaatagaacaaagcaggtaatattgaagactaagagcaaacatgttgctgggaccatcacaaagaagaaaaagaatgtggtggtgggattggttaagggattaccggcgtggcctggccgggaaatgctcgagggtggagagaatttgaggtatttcgggctgaagacggtggagaaaagagtgattgaattcgagtgcaaaagccaaagggaatatgatctttggacacaaggtgtttccatgcttctttccattgcttctgataggaaacataaatgttga

SEQ ID NO: 3 - protein, 453 aa, Arabidopsis thaliana
MDQKSKTIGEQWWRSNPTFKPPETPLDSMEFLSRTWSASATEVSRAVVASPPTSQPPQMRFSEIQNGSSDVTLVPEDEENGIVLGNTFSFASSETSLMVMERIMAQSPEISSPRTSGRLSHSSFTDSPPISPSDIDDFKQFYRVSPSFNGHIRGSSAIPGTAGGSKTVGRWLKDRREKKREETRAQNAQLHAAVSVAGVAAAVAAIAAATASQSSSGTDEQVAKNDSAVASAATLVAAKCVEAAEIMGADREHLASVVSSAVNVRSAGDIMTLTAAAATALRGAAQLKARALKEVWNIAAVIPVDKGTPKGGGGGYRGGELAPVDNFLGICSKELLAKGCELLKRTRKGDLHWKVVSIYINRTKQVILKTKSKHVAGTITKKKKNVVVGLVKGLPAWPGREMLEGGENLRYFGLKTVEKRVIEFECKSQREYDLWTQGVSMLLSIASDRKHKC

SEQ ID NO: 4 - DNA, 1362 bp, Artificial Sequence
  maize optimized NUCPU29 sequence
atggaccagaagtccaagaccatcggcgagcagtggtggagatccaacccgaccttcaagccacccgaaactcctctcgactcgatggaattcctttcgagaacgtggtctgcatccgcgaccgaggtctcacgggctgtggtcgcttcaccacccacctctcagcctccacaaatgcgcttctccgagatccagaacgggtcgtctgacgtcaccctcgtgccagaggacgaggagaatggcattgtgctgggcaacacgttcagcttcgcttcctccgagacctccctcatggtcatggagcggatcatggcgcagtcgccggagatttcgtcaccgcgcacatccggccggctctcacactcttccttcaccgactcgccgccgatttctccgagcgacatcgacgacttcaagcagttctacagagtctcgccatcgttcaacggacacattcgcggctcttctgcaattccggggacagcgggagggtcaaagactgtgggaaggtggctgaaggacagaagggagaagaagcgcgaagagaccagggcccagaatgctcagctgcacgcagcagtttctgtcgctggagtcgctgctgcagttgcagctatagctgccgcgaccgcgtcccaatcttcatccggtacagacgagcaggttgcgaagaacgattctgccgtggcgtcagcggcaacgctcgtggccgccaaatgcgttgaagccgccgaaatcatgggcgcggatagggagcacctggccagcgtggtttcgtcggccgtcaatgtgaggtctgcgggtgatatcatgactctgaccgctgctgcagctaccgcacttcgcggtgcagcacaattgaaggcacgggcgctgaaagaggtgtggaatatcgcggctgtgatcccggtggataagggtacacccaaaggtggaggtggagggtacagaggcggagaactcgcgcctgttgacaacttcctcgggatctgctcgaaggagctgcttgccaaagggtgcgagctcctgaagcgcacgcgcaaaggagatctccactggaaggtcgtgtcgatctacatcaatcggaccaagcaggtcattctcaagaccaagtcgaagcacgtggccggcaccattacgaagaagaagaagaacgtcgtcgtcgggctggttaaaggactgcccgcctggccaggcagagaaatgctggaaggcggcgagaatctccgctacttcggtctcaagactgtcgagaagcgggtgatcgagttcgagtgcaagtcgcaacgcgagtacgacctgtggacccaaggagtgtccatgctcctctccattgcctccgaccgcaagcacaagtgctag

SEQ ID NO: 5 - DNA, 1603 bp, Arabidopsis thaliana
cacgttctctctaacacacacacacactctctctctctttcattagataatggatcaaaaaagtaaaacgattggagaacaatggtggaggtcaaatccaacgtttaaaccgcctgaaacgccattagattctatggagtttttgtcacgtacttggagtgcttccgctactgaagtttcaagagctgtcgtcgcgtctccaccgacttctcaaccgccgcaaatgcgtttctcggagatccaaaacggttcttctgacgtcactttggtgccggaagatgaagaaaacggcatcgttcttggaaatactttttcttttgcttcttcagaaacttctttaatggtcatggaacgtatcatggctcagtcaccggagatttcatcgccacgaacatcagggagactttctcatagctcattcaccgacagtcctccgatctctccctccgacatcgacgactttaagcaattctaccgtgtgagcccttcctttaacggccacatacgtggttcatcagccattcccggcaccgccggagggtctaaaactgttggtcgttggctaaaggaccggcgagagaagaagagagaagagacgcgtgcacaaaatgcacagcttcacgcggctgtatctgtagctggagtggctgccgcggtggctgctatcgctgcagccactgcctctcagtcgagttctggaactgacgagcaagtggccaaaaatgactccgcggtggcttctgccgcgactttggtggcggcgaagtgtgtggaagctgcagagattatgggagctgatcgtgagcacttggcctccgttgttagttctgcggttaacgttcgttctgccggagatatcatgactttgaccgccgctgctgccacagctttgagaggagctgcacaattaaaggcaagagcattgaaggaggtatggaacattgcggctgtgattcctgtagataagggtacaccaaaaggcggtggtggtggttatagaggtggcgagttagcccctgtagataattttcttgggatttgcagtaaagaattgctagctaaaggttgcgaattgcttaaacgcacccgcaaaggtgatcttcattggaaagttgtttcgatctacattaatagaacaaagcaggtaatattgaagactaagagcaaacatgttgctgggaccatcacaaagaagaaaaagaatgtggtggtgggattggttaagggattaccggcgtggcctggccgggaaatgctcgagggtggagagaatttgaggtatttcgggctgaagacggtggagaaaagagtgattgaattcgagtgcaaaagccaaagggaatatgatctttggacacaaggtgtttccatgcttctttccattgcttctgataggaaacataaatgttgaattaaggtatagtcttgggtaaagtcacatgaatatggaaactaatggggcaaatttgtattatctatttttcttggtgtgccttttttctagctagaagagatattgggtctgagctgtctaatttatatttttaaagtaagatctttgtattagaagaatatgtaaattacatgatattcttttaaaggt

SEQ ID NO: 6 - DNA, 1416 bp, Zea mays
atgggcgatccccgggcccgcacgtccaccgtcgccgccggcgagctgcggccgccggagccgccgctcgacccgctcgagttcctctcccgctcctggagcgcctcggggcgcgcgttcgcgccaccgccgccgacgccgccgccggccgcgcacgcgctcgcggccagccccatcgcggaggacccctcgtgcgagtttgacgacggggtcgccgccgccgccgccgcctccgggagctccttctccttcgcctccgcggccacgtcgcagctcatcatggagcggatcctcgcgcagtcccaggaagtggcgccgctcacctccggccggctctcgcacagcagcggcccactcaacggcggcggctctctctccgacagcccgccggtctcgccggagatcgatgacgcaaagtactgcagagcggtgagcacgccgaagccgcaggcctaccgggcgggcaacaagacggtggggcggtggctcaaggaccggaaggagaagaagaaagaggagacgcgggcgcacaacgcgcaggtgcacgcggcggtgtccgtagcggcagtggcggccgccgtggccgccgtggcggcggcgacggccgcggcctcggcgtccggcaaggacgagcgggccgcccgcacggacatggccgtggcgtcggcggcgacgctcgtggccgcgcagtgcgtggaggcggccgagtccatcggcgccgagcgggagcacctggaggccgtggtcgggtcggccgtcaacgtcaggacgcccggagacatcgtcaccgtcaccgctgctgcggccaccgcgttgagaggcgcggccacgctgaaggcgagggctctgaaggaggtgtggaacatcgcggccgtgatcccggtagagaagggcgcggtcggcggcggcagcggcggcggcggaggagggcacaaccagaagcacggcgcgtccaagcagcagcagcatcgcaagctggagagcaacggcagcagcgtcagcgacgtgtcgctggaggaggagaacaacttcctcggcatctgcagccaggagcttctcgctcgcggcaccgagctcctcaaacgcacacggaaaggcgctctgcactggaaggtcgtatcggtgtacatcaaccggatgggcctggtgatgttgaagatgaagagccggcatgtggcggggacgatcacgaagaagaagaaaagcgtggtgatcgacgtgtgcagggacgtgccggcgtgggccgggcggcacctgctggaggacggcgagcaccggcggtacttcggcctgcggaccgcggagcaccgggtgatcgagttcgagtgcggcagccagcgggagcacgagatgtggaccaagggcgtggcgcggctgctgagcatcgccggcgagcggaggcgcctggtggcgtga

SEQ ID NO: 7 - protein, 471 aa, Zea mays
MGDPRARTSTVAAGELRPPEPPLDPLEFLSRSWSASGRAFAPPPPTPPPAAHALAASPIAEDPSCEFDDGVAAAAAASGSSFSFASAATSQLIMERILAQSQEVAPLTSGRLSHSSGPLNGGGSLSDSPPVSPEIDDAKYCRAVSTPKPQAYRAGNKTVGRWLKDRKEKKKEETRAHNAQVHAAVSVAAVAAAVAAVAAATAAASASGKDERAARTDMAVASAATLVAAQCVEAAESIGAEREHLEAVVGSAVNVRTPGDIVTVTAAAATALRGAATLKARALKEVWNIAAVIPVEKGAVGGGSGGGGGGHNQKHGASKQQQHRKLESNGSSVSDVSLEEENNFLGICSQELLARGTELLKRTRKGALHWKVVSVYINRMGLVMLKMKSRHVAGTITKKKKSVVIDVCRDVPAWAGRHLLEDGEHRRYFGLRTAEHRVIEFECGSQREHEMWTKGVARLLSIAGERRRLVA

SEQ ID NO: 8 - DNA, 1378 bp, Zea mays
atgggcgatccgcgggcccgcacgtccgccgtggccgccggcgagctgcggccaccggagccggcgctcgacccgctcgagttcctctcccgctcctggagcgcctccgggcgcgcgctcgcgcccacgccgccgccgccgccggccgcattcgcggccagccccatcgcggaggacgccgcgtgcgagctggacgacgggggcgtcgtcgccgccgcgtccgggagctccttctccttcgcgtccgcggccacgtcgcagctcatcatggaacggatcctcgcgcagtcccaggaagtggcgccgctcacctccggccggctctcgcacagcagcgggcccctcaacggcggcggctccctctccgacagcccgccggtctcgccggagatcgatgacgccaagtactgcagagcggccagcacgccgaaggcgcaggcataccgggcgggcaacaagacggtggggcggtggctcaaggaccggagggagaagaggaaagaggagacgcgggcgcacaacgcgcaggtccacgcggcggtgtccgtggcggcagtggcggcggcggtggccgccgtggccgcggccacggccacggcctcggcgtccggtggcaaggacgagcgcgccgcgcgcacggacatggccgtggcgtcggccgcgacgctggtggccgcgcagtgcgtggaggcggccgaggccatgggcgccgagcgggagcacctggaggccgccgtcgggtcggccgtgagcgtccggacgccgggggacatcgtcaccgtcaccgccgccgccgccaccgcgttgagaggcgccgcgacgctgaaggcgagggctctcaaggaggtgtggaacatcgcggcggtgatcccggtggagaagggcccgggcccggtcggcggaggaggaggaggccaccaccaccaacaccaccagaagcacggcgcgcccaagcagcagctgcaccgcaagatggatagcgacggcagcagcgtcagcgacctctcgctggaggaggagaacagcttccttggcatctgcagccaggagctgcttgctcgcggcaccgaactcctcaaacgcacccggaaaggcgctctgcactggaaggtcgtgtcggtgtacatcaaccggatgggcctggtgatgctgaagacgaagagccggcacgtggcagggacgatcacgaagaagaagaaaagtgtggtggtcgaggtgtgcagggacgtgccggcgtggcccgggcggcacctgctggaggacggcgggcaccggcggtacttcggcctgcgcacggcggagcggcgggtggtcgagttcgagtgccgcagccagcgggagtacgagatgtggaccaagggcgtggcgcggctcctga

SEQ ID NO: 9 - protein, 459 aa, Zea mays
MGDPRARTSAVAAGELRPPEPALDPLEFLSRSWSASGRALAPTPPPPPAAFAASPIAEDAACELDDGGVVAAASGSSFSFASAATSQLIMERILAQSQEVAPLTSGRLSHSSGPLNGGGSLSDSPPVSPEIDDAKYCRAASTPKAQAYRAGNKTVGRWLKDRREKRKEETRAHNAQVHAAVSVAAVAAAVAAVAAATATASASGGKDERAARTDMAVASAATLVAAQCVEAAEAMGAEREHLEAAVGSAVSVRTPGDIVTVTAAAATALRGAATLKARALKEVWNIAAVIPVEKGPGPVGGGGGGHHHQHHQKHGAPKQQLHRKMDSDGSSVSDLSLEEENSFLGICSQELLARGTELLKRTRKGALHWKVVSVYINRMGLVMLKTKSRHVAGTITKKKKSVVVEVCRDVPAWPGRHLLEDGGHRRYFGLRTAERRVVEFECRSQREYEMWTKGVARLL

SEQ ID NO: 10 - DNA, 1020 bp, Zea mays
atgctaactgaacggattgtcgctgttgtggggtggatgcagttctgcagagcggtgagtctgagcacgccgaagccgcagccgtacgcccgcggcgggagcaagacggtggggcggtggctcaaggaccgcagggagaggaggaaggaggaggcgcgggcgcacaacgcgcaggtgcacgccgccgtctccgtggcggccgtggccgcggccatcgccgcagtggcggcgggcaagggtgcgcgcacggacatggcggtggcgtcggccgcgacgctggtggccgcgcagtgcgtggaggccgcggaggccctgggcgccgagcgcgagcagctggcggccgccgtggcgtcggccgtcagcgtcaggacccctagcgacgtcgccaccgtcacggccgccgccgccacagcgctacgtggcgcggcgacgctcagggccagggcgctgaaggaggcgtggaacgtcgcggcggtgacccctgtggagaagggcgcgatgaccggaggcggcgggcatcacctgcacggcggcggctacaagcacgatggccagcagcagcggcagcggctcctcagggaggtggagagcagcaacagcagcagcagctgcttgagcgacgagctggtgctgggcgaggagaacaacttcctaggcatctgcacgcaggagctgctcgcgcgcggcaccgagctgctcaagcgcacccgcaaagggtcgttgcattggaaggtcgtctctgtgtacatcaaccgcgcggggctggtgatgctgaagatgaagagtcggcacgtcggtggcaccatcacgaagaagaagaagagcgtggtggtggacgtgtgccgggacgtggcagcgtggcccgggcggcacctgctggacagcggcgagcaccggcgctacttcggcctgcgcaccgcggagcagcgggtaatcgagttcgagtgcggcagccagcgcgagcatgaggtgtggaccaagggcgtcgcgcgcctcctggccgacgcccggaagcgctga

SEQ ID NO: 11 - protein, 339 aa, Zea mays
MLTERIVAVVGWMQFCRAVSLSTPKPQPYARGGSKTVGRWLKDRRERRKEEARAHNAQVHAAVSVAAVAAAIAAVAAGKGARTDMAVASAATLVAAQCVEAAEALGAEREQLAAAVASAVSVRTPSDVATVTAAAATALRGAATLRARALKEAWNVAAVTPVEKGAMTGGGGHHLHGGGYKHDGQQQRQRLLREVESSNSSSSCLSDELVLGEENNFLGICTQELLARGTELLKRTRKGSLHWKVVSVYINRAGLVMLKMKSRHVGGTITKKKKSVVVDVCRDVAAWPGRHLLDSGEHRRYFGLRTAEQRVIEFECGSQREHEVWTKGVARLLADARKR

SEQ ID NO: 12 - DNA, 1236 bp, Zea mays
atggagcaatgcgccgacgaccacggtgggcaggtcccgaggcttgagggcatcgaggaagaaggcagcccagccgataagtgggctccgacgactgtccggccaccggagacaccgacggagacgatggagttcctggcaaggtcctggagcctctcggcggcagagatctccaaggccctcagggcgctcagctgcggcaatgccgcctccgattctcctcccgctgtcgtcgccacgtccacggcggagcggccgccgcttggcagtgatcatcgccgcctgtctctgccgcagaaggcagacgcggcggctggggctggtggtgaagccggcggaggcacgagccagcccatctctcccagggtgcacctgggcgacgtgaagctgcttcgcgccgcggggagagggaagacggtgggcgcgtggatcaaggagcagagggagaagaagcgcgccgaggcccggtcccgcaacgcgcaggcgtacgcggccacgtccgtggcgggcgtcgcggcggcggtggcggcgctggtggccggcgcggtgttctcgtcgtcctcggcggcgccggagcagcggccggccaagggcgacggcggcggcgccaagaccgcggcggccgtggcgtccgcggcggcgctggtggcgtcgcactgcgtggagatggcgcaggcgatcggcgccagccacgaccagatcctggccgccatccactcggccgtgaacgcgcagagcagcggcgacgtcatggcgctcaccgcaggcgcggccacggctctgcgcggcgccgccatgctgagggcgaggctccacaaggagatccaggcgactgcgttgccaggcgacggcagggagccggagagggacatctcgccgctggtcttcgtctcccggggcggcgagcttctgaagcgcaccaggcagggcgccctccactggaagctcgtcaccgtgtacataaactccggcttcgaggtggttctcaagatgcagagtgcacatatggccggcaccttcatcaaaaccaaaaagtgtgtggttctggacgtgtgctcggagataccggcatgggcaggcagggaggtgggcaggaggggatacttggggatcaggacggtggagagggtgatcgagttcgaggtcaggagcaggcgcgagcagcgcaagtgggtgcagggcatcacggggatgctgcttcgccgcgacagcatcaacaatgccgcctgcttgtaa

SEQ ID NO: 13 - protein, 411 aa, Zea mays
MEQCADDHGGQVPRLEGIEEEGSPADKWAPTTVRPPETPTETMEFLARSWSLSAAEISKALRALSCGNAASDSPPAVVATSTAERPPLGSDHRRLSLPQKADAAAGAGGEAGGGTSQPISPRVHLGDVKLLRAAGRGKTVGAWIKEQREKKRAEARSRNAQAYAATSVAGVAAAVAALVAGAVFSSSSAAPEQRPAKGDGGGAKTAAAVASAAALVASHCVEMAQAIGASHDQILAAIHSAVNAQSSGDVMALTAGAATALRGAAMLRARLHKEIQATALPGDGREPERDISPLVFVSRGGELLKRTRQGALHWKLVTVYINSGFEVVLKMQSAHMAGTFIKTKKCVVLDVCSEIPAWAGREVGRRGYLGIRTVERVIEFEVRSRREQRKWVQGITGMLLRRDSINNAACL

SEQ ID NO: 14 - DNA, 1284 bp, Zea mays
atgcaacacgaagaagtgtgcgatactactacctgcggtgaaggcaatggtcgaggaggtgaggaagaactcgagatggaggggcactcgttgatgaggatgaagaggagggatcacttgctgctcactgacggcgccggcgccggcgccggggcggggcctcttgcggtgcagacgccgatggagcccatggagttcctgtcgcggtcgtggagcgtgtcggcgtcggagatctccaaggtgctggctggcggcgtcggcgggaggcggagctccaacttcgtcgtcgaccgcctgtccgggatgctcatgccggagacgctcgcgctcgcagcctcctcctcctccgccggcaccaacaacatcagcccccgcaagcgcaccctcaggagcaggagcgcgatctccgcgcaccaggtccaccacacggtccacgcgatcggcaagtggttccaccactgggacgcgggcagcagcaaggtggacaaggcccgcgccgagcgggcgcgcgtgcacgccgcggtctccgtggcgagcgtggcggccgcggtcgccgccgtggccgtggccgcgggcgcgagccccgaggtggacgtcaccgacgacggcgccaggatggggagcgcgctggcgtccgcgacgcagctgctggcgtcgcactgcgtcgaggccgccgagctcgcgggggccgaccacgaccaggtggcgtccgccgtggagaccgcggtcgacgtgcggagcccaggggatctgctcaccctcaccgccgcagcggccacagctctcagaggggccgcggcgatgcggcacagggcgcagcgggaggcgaggagcagggcggccgtagcgccgtacgacaaggcctgcagctaccgcgcggacgtctggtgcaaggaaggcgcgctcctcaaacgcaaccgcaaaggcgctctgcactggaagcaggtggccgtgtacatcagcaagaagtcgcaggtgatagtgaagctgaagagcaagcacatcggcggcgccttctccaagaagaagaaaggcgtcgtctacggcgtctacgacgacatcccggcgtggccggcgcgcgagggcgtcgccgcgcaaagctcggcgccggagacgtgccacttcgggctgaggacggcgcatggcctgctcgagttccagtgcgaaagcagggcacagaggcgggactgggtggaggcggtgaaggacctgatccggcaggtggccggcgggactgctctgctcgagcactccttcgagtcgctaaggctgagcgcgtcttga

SEQ ID NO: 15 - protein, 427 aa, Zea mays
MQHEEVCDTTTCGEGNGRGGEEELEMEGHSLMRMKRRDHLLLTDGAGAGAGAGPLAVQTPMEPMEFLSRSWSVSASEISKVLAGGVGGRRSSNFVVDRLSGMLMPETLALAASSSSAGTNNISPRKRTLRSRSAISAHQVHHTVHAIGKWFHHWDAGSSKVDKARAERARVHAAVSVASVAAAVAAVAVAAGASPEVDVTDDGARMGSALASATQLLASHCVEAAELAGADHDQVASAVETAVDVRSPGDLLTLTAAAATALRGAAAMRHRAQREARSRAAVAPYDKACSYRADVWCKEGALLKRNRKGALHWKQVAVYISKKSQVIVKLKSKHIGGAFSKKKKGVVYGVYDDIPAWPAREGVAAQSSAPETCHFGLRTAHGLLEFQCESRAQRRDWVEAVKDLIRQVAGGTALLEHSFESLRLSAS

SEQ ID NO: 16 - DNA, 1764 bp, Sesbania bispinosa
acctctctagacataagactacccctcacaatagcacatacctgacatggaaccaaagtgcacaatcaattgtcggcggccggagtatcttccaaccggcggcacacctctgccggagagtcctcgagtgccaatggagtttctgtcaaggtcatggagtgcctcagctcttgaggtttccaaagctcttgcacctcatcaacctcaacctcaaccacaaccacctcttccttcatgcatgccttctaaacctgtaaatgggtcccatagtgggagtactactaattctattcctgaagaaaccgttggccactctatctttgaggagttctctaagaatcagttctctttcgcttcctctgctacctctcagcttgtccttgagcgcattatgtcacagtccataagagaggaagtgtcgccattaacatcaggtaggctatctcatagcagtgagcctttgaacggtggtggttcattagctggaacagatagccctccaatttcaccttcagatgaatttgacgatgtcgttaagttttttcgagcaaacaattccattcacccgttattcaatggtggacgagccagtgctgctactggcaatggcatggcttgctcaggacctaaaagtaaaactgttggaagatggttgaaagacagaagagaaaagaagaaagaagaaaacagaacccataatgctcagctgcacgctactatttcagtggctgcagtggcttcatctatagcagccattgcagctgcaacagcagcctcatctacacccaacaaggatgagaaaatggccaagacagacatggctattgcttctgcagccacattagttgcagcacagtgtgtagaggctgctgaagcaatgggagctgagcgtgaccaccttgcttctgtggtcagctcagctgtaaacgtccattctcatgatgatatcactactctcacagccgcagctgccacagcactaagaggagcagcaaccttaaaagcaagagctctaaaggaagtatggaatattcctgcagcaacaccactagagaaaggcattggggggattgcaatacgtggtaaaagtaacaataacagcaacaccagcaatacaagcaacagtgatagtggtgaaattgtcaatgcagaaaactttctaggtacctgcagccaagagctacttgctaagggcagtgaattactcaaacgcacccgtaaaggtgatttgcactggaaaatagtttctgtttacatacatcgaacaggacaggtaatgctgaaaatgaagagcagacatgttgccggaaccattaccaaaaagaaaaagaatgtagtgttcgacatatgtacggatttaccagcatggccagggagacatctattggaggatggagaaaaacgacgctacttcggattgaaaacagagtcaagagggatggtggaattcgagtgtagaaatcaaagagaatacgacatctggactcaaggcgtttcgagacttctctccatcgttgcacaaaaacgacaaaacagatacgacaactgaattaacctctcatatagtactaattagtctccaccacccattcctcactatatgtattgagatgtatgtattgtttcgctgttgccataatgtttggattgatcaatttagttgactgcagtttctatccggatccataatcaaaccaactatagcacttacattaaatcaataaaccatttcagttataactcagccaaaaaaaaaaaaaaaaa

SEQ ID NO: 17 - protein, 500 aa, Sesbania bispinosa
MEPKCTINCRRPEYLPTGGTPLPESPRVPMEFLSRSWSASALEVSKALAPHQPQPQPQPPLPSCMPSKPVNGSHSGSTTNSIPEETVGHSIFEEFSKNQFSFASSATSQLVLERIMSQSIREEVSPLTSGRLSHSSEPLNGGGSLAGTDSPPISPSDEFDDVVKFFRANNSIHPLFNGGRASAATGNGMACSGPKSKTVGRWLKDRREKKKEENRTHNAQLHATISVAAVASSIAAIAAATAASSTPNKDEKMAKTDMAIASAATLVAAQCVEAAEAMGAERDHLASVVSSAVNVHSHDDITTLTAAAATALRGAATLKARALKEVWNIPAATPLEKGIGGIAIRGKSNNNSNTSNTSNSDSGEIVNAENFLGTCSQELLAKGSELLKRTRKGDLHWKIVSVYIHRTGQVMLKMKSRHVAGTITKKKKNVVFDICTDLPAWPGRHLLEDGEKRRYFGLKTESRGMVEFECRNQREYDIWTQGVSRLLSIVAQKRQNRYDN

SEQ ID NO: 18 - DNA, 1961 bp, Amaranthus hypochondriacus
aattgccaaacacctccttccatctcacacacaaaatacataccaattaccaagtactccactcctaccagtacaatctccagtagagggccccacctaacaagtccaaaaaaaaaaaaaaaaaaatacccatagatcaagtaaccacctccatggacagaggagtagccgtcggtgaaccatggcgacccgacccgacagtaagatccgacttgtttaaacctcctgaaactccgaaagagcccatggagtttctctcacggtcttggagtgtttcagctctagaagtttctaaagcgcttactccttctaaaatgatttttcctaagggtatggttggcggcggcggtagtggtagtggaggcggcggtggtggtggtgatgggaccattttggaagatgttgcggaggaagtggcggcggttgttagtggaaatccgttctcattcgcttcttctgaaacttctcagcttattatggagcggattatgtcacaatctcaagaggtatctccgcggacttctggtcggctttcgcatagtagtggcccactcaatggctccttgaccgatagtcctccggtgtctccctccgaaatggacgatgtcaagtatggtcgcctaaacaaccacgcaaattcacagtatcgatcaactttcaatcctacagcagtaacccatccaactgcgacagggaagactgtcgggagatggcttaaagaccggagagaaaaaaagaaggaagaaacgagagctcataatgctcagctacacgctgcagtttctgtggcgggtgtcgcagcagctattgctgcaatggcagctgcgactgctgcaacgtcaggaagtcgtaaggaccaagaagctgcacggactgacatggctgttgcatctgctgctacccttgtggctgctcaatgtgttgaggcggctgaagcaatgggtgccgacagggagcatttagcttctgttgttagctctgcggttaatgtcggatcagctggtgatatcatgacgttaacggctgctgcagccaccgcgttgcgtggagctgcgacactgaaagcgagggcattaaaggatgtgtggaacattgcagccgtgataccagccgagaaagggatcactgaaagcaatggtagcaatggttctagtggcagtcacagtggtgaactgttgcccgaggagaatttccttgggatctgcagccgagagctacttgctcgaggctctgagcttttgaaacgcactcggaaaggcgatcttcactggaaaatagtgtccgtttacatcaatagacttaaccaggttatactaaagatgaagagtaggcacgttgctgggaccatcacaaagaagaaaaagaatattgtaatcgaagtcatcaaagatatgccggcttggcctggccgccatttgttggagggcggagattacagaagatatttcggattaaaaacgctcttacgtggtgttgtagagtttgaatgtcgcgaccaaagggaatacgagatttggaccgaaggagtctcaaggctacttgctctttccgccgaaaagaacaataggcaaaggatctaggagacttagtactaccaatatatttttgggattaatgtgtttaatgggttcctctcttgaagacccttcttgagccggggactctttcgatcgccctctctttatgggtatgagacgtcgtctatcttccctccccagacccagaacatagatttatctatgttcaggatacaccgggtatgatgatgatgatgatgatgatgatgaaatgtaatttgttactacctctattcagcctaaatgccccatttagttttttgcactattcactcatcactcttaatttgtgttttactcttaatttataagttaaaacatagtcatgtgggatcttgttttattcgtctcaatacaaggattattaataaaaaaaaaaaaaaa

SEQ ID NO: 19 - protein, 475 aa, Amaranthus hypochondriacus
MDRGVAVGEPWRPDPTVRSDLFKPPETPKEPMEFLSRSWSVSALEVSKALTPSKMIFPKGMVGGGGSGSGGGGGGGDGTILEDVAEEVAAVVSGNPFSFASSETSQLIMERIMSQSQEVSPRTSGRLSHSSGPLNGSLTDSPPVSPSEMDDVKYGRLNNHANSQYRSTFNPTAVTHPTATGKTVGRWLKDRREKKKEETRAHNAQLHAAVSVAGVAAAIAAMAAATAATSGSRKDQEAARTDMAVASAATLVAAQCVEAAEAMGADREHLASVVSSAVNVGSAGDIMTLTAAAATALRGAATLKARALKDVWNIAAVIPAEKGITESNGSNGSSGSHSGELLPEENFLGICSRELLARGSELLKRTRKGDLHWKIVSVYINRLNQVILKMKSRHVAGTITKKKKNIVIEVIKDMPAWPGRHLLEGGDYRRYFGLKTLLRGVVEFECRDQREYEIWTEGVSRLLALSAEKNNRQRI

SEQ ID NO: 20 - DNA, 1710 bp, Artemisia tridentata
gaggcatttaaacaacactacacgcttacagaaacactagaccccttccaatcagtcactaaaaaacccaccgtcgtcgtacactacacacaaactgtcactaacccacttcacaccgtcactatctcattttttcaccattactacattctcaaaaaaaaaaaaaaaaaaaaaactcacaacgcattaatggactcattaagccacgtcatattcaggccaccagagacacccaatgagcccatggagtttttgtcacgttcttggagcgtttctgctttgcaagtcagtaaagctttagctccgtcgcaaagtggtggaggtgatgaaggtgacaaagtttgtggtgagatggaagaagttgctaatggtggtggtaacccttttgcttttgcttcatctgagacttctcagcttattatggagagaatattggcacaatctcaagaggtatcaccaagaacttcagggaggctgtcacacagtagtggccctcttaatggttcactcacagatagtccacctgtttcaccttctgaacttgatgatatgaagtacggtcgattaaagaaccctgtaaacacccagtatcgaggtggggctacaagtaccgctgcgaccactggtggcgggaaaacagtgggaagatggttaaaggacagaaaagagaaaaagaaagaggagacaagggcacataatgcacagttacacgcaactgtttcagttgctggtgttgctgctgttgttgttgcacttgcagctgctactgccgcctcatcaggacaaagaaaagatgaggagatgatgaaaactgacttggcggttgcatctgctgccaccttggtggcggctcagtgtgtggaagctgcggaggctatgggtgctgaacgtgaccatttggcatcggttgttagctctgcggttaatgttcgatcacctggcgatatcatgactttaacagctgctgccgctactgctcttcgtggtgcggcgacattaaaagcaagggcagtgaaggacacacgcaatatcccggctgtaccccgcacagataaagggacaaatagcactggaactggcagcagttcaaatggtagttttagtgacgagtttgttcctgaagaaaattttcttggcatatgtagccgagaactacttgcaagaggttgtgtgctccttaaacgcacacgtaaaggtgatcttcactggaaaatagtttctgtttatatcaacaaaatggatcaggtgatcctgaagatgaagagtagacatgttgctgggaccattacaaaaaagaagaagaatattgtgttggaagttttgaaagatatgtcaccttggccgggtcgccaccttgtggaaggtggtcatgaccggtgttactttgggctaaagactgtggaccgtggagttgtcgagtttgaatgcaaaaatcaaagggagtatgatatttggactcaaggtgtgtgtaggttgcttgcggttgttacagatagaaacaatgggatttaaatcttggttagctttttgtgggtttgaattagctatctttatatttgtaaatcttttggcttagtatggtatgatcattgtggtttattttttgaacggttttagttggtaatggtttagtttttgcgacgttggtgttattgctgcttttgtaaatttatgtttggatataattactaaaaaaaaaaaaaaaaaaaa

SEQ ID NO: 21 - protein, 440 aa, Artemisia tridentata
MDSLSHVIFRPPETPNEPMEFLSRSWSVSALQVSKALAPSQSGGGDEGDKVCGEMEEVANGGGNPFAFASSETSQLIMERILAQSQEVSPRTSGRLSHSSGPLNGSLTDSPPVSPSELDDMKYGRLKNPVNTQYRGGATSTAATTGGGKTVGRWLKDRKEKKKEETRAHNAQLHATVSVAGVAAVVVALAAATAASSGQRKDEEMMKTDLAVASAATLVAAQCVEAAEAMGAERDHLASVVSSAVNVRSPGDIMTLTAAAATALRGAATLKARAVKDTRNIPAVPRTDKGTNSTGTGSSSNGSFSDEFVPEENFLGICSRELLARGCVLLKRTRKGDLHWKIVSVYINKMDQVILKMKSRHVAGTITKKKKNIVLEVLKDMSPWPGRHLVEGGHDRCYFGLKTVDRGVVEFECKNQREYDIWTQGVCRLLAVVTDRNNGI

SEQ ID NO: 22 - DNA, 1452 bp, Delosperma nubigenum
aaaaagcttagatttcatgcttatgaagttggtttatttacctctttcttctatggtgacagagatgggttctgatgccatgaaatccgcggctaataagctcgagaacatagacgaaaatgggccggctaattggctaccggcttctgttcctccaccggagaccccaactgagtccatggagttcttggcaagatcatggagcgtttcggcaatagagctaaccaaggctctttctaccaccacccataatgtgccaaaagatgtcgggaagacaaggttcagtttttttgacgaggagctggatatcgtgagtcctgtgctgtcaaaggattataaagtggaatcaatgggaaatggagacagtcctccagtttctccaagaggaagtgatgatacaaaggaactctattttcttcatcaagcacttcaacctgacttcttatacaatcaacagttgctcaaaagtgggctatgcaagagtataataaaaggaaagacaatggggagaaggattaaggatcaaagggagaggaaaaagcaggaagcaagaactcacaatgctcagcttcatgctgcagtgtcagtagcaggtgtagcagctgctgttgcagcattgacagcctcaaatgcaaccgttgcggaaatgtcaagtggacaatcaaagacaccttctagtaaccctgctgctatggcatctgctgcagcacttgtagcttcacactgcatcgagattgcacaggaaatgggggctgatcaggatcagatcctggcagctgttaattctgcaataaatgcaagaaccaatggtgatatcatgaccttaacagcaggagcagccactgcgctacgaggggctgcgaccttgaaggcaaggcttcagaaggggcatggagcagcagcgcttgctctaggagaggagcaagctgaagacggtagagagttaaactcgatgactgctatgagcttcattttgcagggtggagaacttctaaagcgtaccagaaatggtgatttacactggaagcaagtttccttcagcttagattcaacattgcagataattgttaaaatgaaaagcagacacgtagcaggggcattcacaaagaagaagacatttatagtatctggagtttactctgatattccagcatggcccggaagagacctggaagacgaagctgagcagagggcttactttggaatacaaacagctgacagggttatcgaatttgaatgtaaaaatagatgtgaaagacaaatgtggacagatggtattcaccatctcttgaattgccgcattaccatgccatttccgtaagagtagggttatatatggactggtgtaggactttgatctttgtaacgaaatcgacatttaaaaatatgaaatctcattgacatatgtaaatgatgatgacaaaatcgttgatcaaaaaaaaaaaaaaaaaa

SEQ ID NO: 23 - protein, 434 aa, Delosperma nubigenum
MLMKLVYLPLSSMVTEMGSDAMKSAANKLENIDENGPANWLPASVPPPETPTESMEFLARSWSVSAIELTKALSTTTHNVPKDVGKTRFSFFDEELDIVSPVLSKDYKVESMGNGDSPPVSPRGSDDTKELYFLHQALQPDFLYNQQLLKSGLCKSIIKGKTMGRRIKDQRERKKQEARTHNAQLHAAVSVAGVAAAVAALTASNATVAEMSSGQSKTPSSNPAAMASAAALVASHCIEIAQEMGADQDQILAAVNSAINARTNGDIMTLTAGAATALRGAATLKARLQKGHGAAALALGEEQAEDGRELNSMTAMSFILQGGELLKRTRNGDLHWKQVSFSLDSTLQIIVKMKSRHVAGAFTKKKTFIVSGVYSDIPAWPGRDLEDEAEQRAYFGIQTADRVIEFECKNRCERQMWTDGIHHLLNCRITMPFP

SEQ ID NO: 24 - DNA, 1257 bp, Linum perenne
aataaacaaatcccccaaactcactcactctctctctctatctcctccttccttgctccaatggacaagtccttcaccgatatttggcggccggacccgtccatcttcttccgcccgccggaaacccctcgtgagcccatggaattcctctcccgatcatggagcccttccgccttagaggtttccaaagctctgaccccaccaccacctccctctatgctcctctccaaatcttccgccatccaagaagacatcgccggtgagcttgacgacactgcctcctttgccggaaaccctttctcctttgcctcttcggagacatcccagatggtcatggaccgtatcttgtctcagtcgcaagaggtgtctccaagaacttccggcaggctgtcccacagcagtggtcctctcaacggttcattaaccgacagccctccggtgtctccgtcggagattgaagacgtcaagagctgccgccccatcaacactaaccacaacaacctcagtttcaacctcctcaacactcatttccggacggcctccggcacgccagctggcggcgcgacattcggcggcactaatggcggcgggaagacggtagggaggtggttgaaggataggaaggagaagaagaaagaggagacgagggcccacaatgctcaggtccatgcggccatcaccgtcgccggagtcgcctccgccattgcggctatagctgccgccaccgccgcttcttcaggcaaagacgagcagcacgccaagactgacatggctgttgcctccgccgcgactttggtggcggcgcagtgcgtagaggcggcggaggcgatgggggcggaacgagaccatctcgcctccgtcgtcagctctgccgttaacgttcggtctcccggcgacatcatgaccctcaccgccgcagcagccacgtctttacgtggggcagcgacattgaaggctagggcaatgaaggacgtatggaacatagcggcggcgattccagcggagagaaggattggcggcggtgctggtggcagtggtggtagtaattgtagttcttctagcggcgagcttgtccctgaagagaattttctagggatttgcagtagagaattgctagccagaggttgtgagctactcaaacgaactcgtaaaggggacctgcactggaagattgtttctgtttacattaacaagtcgaatcaggttatgttgaagatgaagagtaggcatgttgctgggaccataaccaaaaaaaaa

SEQ ID NO: 25 - protein, 399 aa, Linum perenne
MDKSFTDIWRPDPSIFFRPPETPREPMEFLSRSWSPSALEVSKALTPPPPPSMLLSKSSAIQEDIAGELDDTASFAGNPFSFASSETSQMVMDRILSQSQEVSPRTSGRLSHSSGPLNGSLTDSPPVSPSEIEDVKSCRPINTNHNNLSFNLLNTHFRTASGTPAGGATFGGTNGGGKTVGRWLKDRKEKKKEETRAHNAQVHAAITVAGVASAIAAIAAATAASSGKDEQHAKTDMAVASAATLVAAQCVEAAEAMGAERDHLASVVSSAVNVRSPGDIMTLTAAAATSLRGAATLKARAMKDVWNIAAAIPAERRIGGGAGGSGGSNCSSSSGELVPEENFLGICSRELLARGCELLKRTRKGDLHWKIVSVYINKSNQVMLKMKSRHVAGTITKKK

SEQ ID NO: 26 - DNA, 1620 bp, Linum perenne
aaccttattattacactccctctccctccgactcccaccactccaataacaacaacatattattaaacacggactctccctccttctttcttaatggacaattccttgttccgggcgccggaaacccctcgtgagccaatggagttcctctcccgttcatggagtgtatcagctttcgaggtctccaactcactacctcctcaaatgatcctctcctcctcctcctccaattccaaagacataatagctgagcttgatgaaggcggcaccgccattttctcccgcaaccctttctccttcgcttcctccgacacttcccagatgctcatggaccgtatcttctctcactcgcaagatgtgtccccaagaacttcaggcaggctctctcatagcagcggtcctctcaatggttccttaaccgacagccctcctgtttctccctctcatcaaatcgaggatgccaggggcttccgcggtagcaacaacaacagcatccattacaatctccacagcgcccagtaccgtgcgccatccggcacgccacgtggcagcgggaagacggtagggaggtggttaaaggataggaaggagaagaagaaggaagagacgagggctcacaatgctcaggtccacgctgctatatccgtcgccggagtggctgcagccatcgctgctatagcagctgccacagcagcgtctacagggagcggcaacgacgagcagcacgcccggaccaacatggccgtggcgtcagctgcgactctagtggctgcgcagtgcgttgaggcggcggaagctatgggggcagagcgcgagcatcttgcctctgtgatcagctccgccgtgaacgtaaggtctcccggtgatatcatgacccttactgccgcagcagccacatctctgcgtggggcagcaacattgaaggcgagggcattaaaggaagtatggaatgtagcggctgtgattccattagagagagggattaccgttgttggtggtggtggtagtaatggcagttctagcggagagcttgttcctgaagagaattttctagggatttgcagcagagaattgttagccagagggtgtgagttactgaagcgcacaaggacaggggaactgcactggaaggttgtatctgtttacatcaacaggatgaatcaggttatgttgaagatgaagagcagacatgttgctgggaccataaccaaaaagaaaaagaatctggtgttggatgtgataaagaatgtaccagcatggccgggtcgtcatttgctggagggtggagagaagaggcgatactttggattgaagacagtgttaagaggggaggttgagtttgagtgcatgagtgacaaggaatatcatctgtggactgagggagtgtcaaggcttctagccattgctggacaaaagagtaatacaccaccctatagccataggaactgaaatatacctataactctggcgggttctttctatatgtatatggtaaatcataaaaacaaagggtggggggttgaggacaaagaaggatacgattattgtccatatgatgcaaaagggggatttgtttattggtattattctaacagtgaaagtgaaactagtttgaatgag

SEQ ID NO: 27 - protein, 451 aa, Linum perenne
MDNSLFRAPETPREPMEFLSRSWSVSAFEVSNSLPPQMILSSSSSNSKDIIAELDEGGTAIFSRNPFSFASSDTSQMLMDRIFSHSQDVSPRTSGRLSHSSGPLNGSLTDSPPVSPSHQIEDARGFRGSNNNSIHYNLHSAQYRAPSGTPRGSGKTVGRWLKDRKEKKKEETRAHNAQVHAAISVAGVAAAIAAIAAATAASTGSGNDEQHARTNMAVASAATLVAAQCVEAAEAMGAEREHLASVISSAVNVRSPGDIMTLTAAAATSLRGAATLKARALKEVWNVAAVIPLERGITVVGGGGSNGSSSGELVPEENFLGICSRELLARGCELLKRTRTGELHWKVVSVYINRMNQVMLKMKSRHVAGTITKKKKNLVLDVIKNVPAWPGRHLLEGGEKRRYFGLKTVLRGEVEFECMSDKEYHLWTEGVSRLLAIAGQKSNTPPYSHRN

SEQ ID NO: 28 - DNA, 1666 bp, Triglochin maritimum
actggtttcgtctcgtccctctcctctcctctcctctcctctcctctcccctcctataaatccaaacccagcaatgctccagaggaagtagaagcaacccatgatggaggaagacatccccaacgacccaatggagttcttctcccggtcttggactctctctgctctcgacatctccaaagcccagaaagactccacctttggacatggaaatggaaatggaaatggtgttatacctgaggaggagattggggttgaggccacttccttctcctttgcttcctctgccacttcccagcttgtcatggacaggatcatgtcccagtcgcaagaggtatcacctctgacatctggaaggctctcccacagcagtggtcctctcaatggtggcggatctctcacagacagccctccactctccccctcagccattgatgatgtcaagtactgcagagggaacaatgccctgaagccgcaataccgtgggggtagcaagaccgtcgggcgatggctgaaagacaggaaggagaagaaaaaggaggagactcgtgcgcataatgcccaattacacgctgcggtttctgtggctggtgtggctgctgccgttgctgcaattgcggctgccacagcggccgcttctgctcccaggaaagatgaacaggcagccaaaaccgacatggctgtggcttctgctgccacacttgtggcggcacaatgcgtggaaatggcagagtcaatgggagtcgaacgtgaacatttggcgtcggttgtccattctgccgttaatgttcgaactccgggggatatcaatactctaactgccgctgccgccacagcactaaggggcgccgtgacgttgaaggcaagggctcttaaggatgtttggaatatagctgctgtaattcctgtggagaagggtggattgggaggcaatatgcatcaccagcgccgtcgagaccaaaattctaatgctgatgatcgaagttcaagcaatgagctcatcccggaggagaatttcctcggtttatgcagccgagaattgcttgctcgtggcaccgagctcctcaaacggacacgaaaaggcgatctccactggaaaattgtatctgtctacattaacaaaatgggccaggttatgcttaagatgaagagcagacatgttgcaggaacaatcactaagaagaagaagaacgtggttgcggaggtatgcaagggcatccaagcatggccagggaggcatttacttgaaggcggcgagcaacggcggtacttcgggttgaaaacagtagacaaaagggtgatagaattcgaatgcaggagcccgagggagcacgagatatggacgcgaggcgtcacgcaccttctgaacatcgttgcagagaggaaaggcactcggtcacgattgacggagaaataagtgtaaattcacagatgtttcggaacttgaaggtgtgtttttgctgcagtagttgggattagtgggatgatgagaagaatggttggggtgttttggtaaatgaatgtgtttagtgtggtttgttagtcgtgtataaatcaagaagttgtgtaagtctagtttttttactgagtgtgatgttcttttgtcagttgacatgtttgaaaatgttggatttttttgttttaaaaaaaaaaaaaaaaaaaaaaa

SEQ ID NO: 29 - protein, 438 aa, Triglochin maritimum
MMEEDIPNDPMEFFSRSWTLSALDISKAQKDSTFGHGNGNGNGVIPEEEIGVEATSFSFASSATSQLVMDRIMSQSQEVSPLTSGRLSHSSGPLNGGGSLTDSPPLSPSAIDDVKYCRGNNALKPQYRGGSKTVGRWLKDRKEKKKEETRAHNAQLHAAVSVAGVAAAVAAIAAATAAASAPRKDEQAAKTDMAVASAATLVAAQCVEMAESMGVEREHLASVVHSAVNVRTPGDINTLTAAAATALRGAVTLKARALKDVWNIAAVIPVEKGGLGGNMHHQRRRDQNSNADDRSSSNELIPEENFLGLCSRELLARGTELLKRTRKGDLHWKIVSVYINKMGQVMLKMKSRHVAGTITKKKKNVVAEVCKGIQAWPGRHLLEGGEQRRYFGLKTVDKRVIEFECRSPREHEIWTRGVTHLLNIVAERKGTRSRLTEK

SEQ ID NO: 30 - DNA, 1700 bp, Tradescantia sillamontana
attctgagctcaaaaaaacactctcataccctaattgtagcaccattgatggaggactccattgagctcacaaaggatcccatggagttcctctcacgttcatggagtgcctcttcacttcatgtcacaaggcctctctctccaccaccttctacttttcccactgcctctattcctgaggaggagcctgatagctcagctgtttcagctgcaccaatttcatcctctgctgctgcacctggaaatcctttcacctttgcgacatcgacaacttcgcagatagttatggagagaataatgtcccaaactgaagtgtctccactgacttctggccggttgtcacacagtagtgggcctctcaatggtggtggatcactctccgatagtcctccggtttcgccttgtgacctcgaagatttcaagtattctcgtgctagtaatactccaaagccacaggcataccggaccaataagactgttggaaggtggctgaaggacagaagagaaaagaagaaagaggagactagggcccagaatgcccagcttcatgctgccgtttctgttgccggtgtggcagcagctgttgcggctattgcagctgcaactgctgcagcctcagggtctggcaaagatgacagagctgcaaggattgacatggcagtcgcatcagctgcaactcttgtagctgctcagtgtgtggaggctgctgaagctatgggagctgagcgcgagcatctggcatcagtaattggttcagctgttagcgtaaggacaccaggggatattactactcttaccgctgctgcggccactgctctacgtggagctgcaactttgaaggccagaaccctcaaggaggtctggaatatagctgcagttattccggtggaaaaggggagtttgacaaatcacaaaattcaccagcagaccaagcaacaaaaggaggtggaaagtaacagtagcagtttcagtgatgatcttgtccctgaggaggagaacttcttgggactatgtagccaagaattgcttgctagaggcactgagcttcttaaacggacgagaaaagggtcacttcattggaaatttgtctcagtttacattaaccaagtgggtcaggtaatgctcaagatgaagagtaggcatgttgcagggacgataaccaagaagaaaaagaatgttgtgatagacgtttgtaaagacatccaagcctggcctggtaggcactggctggaagggggagagcaacgtagatactttggattgatgacagcggaaaaacgagtcatagaattcgagtgccgaagtcggagggaatatgaaatgtggactcgtggggtgtcgcgtctgctcaatattgtcaatgagaagaagcacctctcttgaccatgagctgccgacactagccagctgatcaaagttattattatctatatggaaagatgctgcatgcatgacccacccgaagttaagagcttgacaagaatgtttccaatctggaattcatccagtgggtttccatctcttattgttatagagttagataatcaggttaaattaaagatgctgtttgctaggtgtttacttctgttttctggtttgtttcgaactacgtggggttgtatatctactaacagtgaagggaaaaagaaaaaaaaaagttaaaggtattcagttatgaaaaaaaaaaaaaaaaaaaaaaa

SEQ ID NO: 31 - protein, 444 aa, Tradescantia sillamontana
MEDSIELTKDPMEFLSRSWSASSLHVTRPLSPPPSTFPTASIPEEEPDSSAVSAAPISSSAAAPGNPFTFATSTTSQIVMERIMSQTEVSPLTSGRLSHSSGPLNGGGSLSDSPPVSPCDLEDFKYSRASNTPKPQAYRTNKTVGRWLKDRREKKKEETRAQNAQLHAAVSVAGVAAAVAAIAAATAAASGSGKDDRAARIDMAVASAATLVAAQCVEAAEAMGAEREHLASVIGSAVSVRTPGDITTLTAAAATALRGAATLKARTLKEVWNIAAVIPVEKGSLTNHKIHQQTKQQKEVESNSSSFSDDLVPEEENFLGLCSQELLARGTELLKRTRKGSLHWKFVSVYINQVGQVMLKMKSRHVAGTITKKKKNVVIDVCKDIQAWPGRHWLEGGEQRRYFGLMTAEKRVIEFECRSRREYEMWTRGVSRLLNIVNEKKHLS

SEQ ID NO: 32 - DNA, 1561 bp, Tradescantia sillamontana
attccactcaactaccaaccaccttcaccataactaccagcaatggcagaaaccaaaaaaccaaacccgccggagctccgacgccggccgaagtcactcagagacgctgccgcttcgacggagctcccagacaacccaatggagttcctcgcgcgttcgtggagcgcttcggctctccagctttccaaggctttcaatgctaagcctcagctgcgcctcacgtcgccggtgatcgccggaattgatgaagatctcgccggagaagcggaggaggaggaggaggagaatacggtggccggaaatcctttcgcgtttgcttctgctgcgacttcgcagcttgttgttgagaggattatgtctcagtctgaggtcactccaccgacttctgggagaccatctcacagtagtgggcctttcaattgcggtggtggcggcggagaatcgccctccgatagcccgccgctatcgccatctgatatggaggaaatcaagcaatgccgagctagcaacacacagaaacatcatccttataaagggactaacaagactgtaggaaggtggctgaaagaaagaagggagaagaagaaagaggagacgaggaccctgaatgcacagctccatgctgcagtctcggtggctagtgttgctgctgcagttgcagccattgctgctgccactgcagctgcatccggagacaaagatgaccgaacagccaggaccgatatggcagttgctgcagctgcaacgctcgtagcagctcagtgtgtggaagctgcggaatctatgggggctgagcgcgagcacttgacatcagttgtcagctcagctgttaatgtcaagactccaggggatatcattactcttacagcagctgctgccaccgctttacgaggtgctgctactctgaaagcaagagctctcaaggaggcatggaatgtagctgcagttataccagtcgagaagggagaagcaggtaatcactataatcatcggcagaataagagacaagagaagggattgcatggcagtaacagcatctctgccgacgagactgttcccgatgaaaagaagtatttgggtctttgttatcaggaactccttgccagaggcagtgaactacttaaacagactagaaaaggtgccttacattggaagatcatatcggtttacataaatcgaatgaatcaggtaatacttaagatgaagagtaggcacgttgcagggaccatagttaaaaagaaaaagtttgttgtgattgaagtttgcaaggacattaaagcatggccaggccgacacttattggagggtggcgatcagcgacgatactttgggttgaagacagcagagcacagggttatcgagtttgagtgcccaagccgaagggaatacaagatgtggactcaaggagtttcccgactgctcaacattgtcgaggagaggaagcatcgaaactgaacttattaatttgtttggtgaaatggtgtcgtgattttctacctctattaataaattagtcggctaaatgttgtaaattagggaaaaaaaaaaaaaaaaaaaaaaa

SEQ ID NO: 33 - protein, 470 aa, Tradescantia sillamontana
MAETKKPNPPELRRRPKSLRDAAASTELPDNPMEFLARSWSASALQLSKAFNAKPQLRLTSPVIAGIDEDLAGEAEEEEEENTVAGNPFAFASAATSQLVVERIMSQSEVTPPTSGRPSHSSGPFNCGGGGGESPSDSPPLSPSDMEEIKQCRASNTQKHHPYKGTNKTVGRWLKERREKKKEETRTLNAQLHAAVSVASVAAAVAAIAAATAAASGDKDDRTARTDMAVAAAATLVAAQCVEAAESMGAEREHLTSVVSSAVNVKTPGDIITLTAAAATALRGAATLKARALKEAWNVAAVIPVEKGEAGNHYNHRQNKRQEKGLHGSNSISADETVPDEKKYLGLCYQELLARGSELLKQTRKGALHWKIISVYINRMNQVILKMKSRHVAGTIVKKKKFVVIEVCKDIKAWPGRHLLEGGDQRRYFGLKTAEHRVIEFECPSRREYKMWTQGVSRLLNIVEERKHRN

SEQ ID NO: 34 - protein, 475 aa, Arabidopsis thaliana
MEKLMVPTWRPDPVYRPPETPLEPMEFLARSWSVSALEVSKALTPPNPQILLSKTEEEEEEEPISSVVDGDGDTEDTGLVTGNPFSFACSETSQMVMDRILSHSQEVSPRTSGRLSHSSGPLNGSLTDSPPVSPPESDDIKQFCRANKNSLNSVNSQFRSTAATPGPITATATQSKTVGRWLKDRREKKKEETRAHNAQIHAAVSVAGVAAAVAAIAAATAASSSCGKDEQMAKTDMAVASAATLVAAQCVEAAEVMGAEREYLASVVSSAVNVRSAGDIMTLTAGAATALRGVQTLKARAMKEVWNIASVIPMDKGLTSTGGSSNNVNGSNGSSSSSHSGELVQQENFLGTCSREWLARGCELLKRTRKGDLHWKIVSVYINKMNQVMLKMKSRHVGGTFTKKKKNIVLDVIKNVPAWPGRHLLEGGDDLRYFGLKTVMRGDVEFEVKSQREYEMWTQGVSRLLVLAAERKFRM

SEQ ID NO: 35 - protein, 472 aa, Arabidopsis thaliana
MEKPVFAPWRSDQVFRPPETPLEPMEFLSRSWSVSAHEVSKALTPSQQLLSKASIENTTVILEEPIAAGETETEDNSFVSGNPFSFACSETSQMVMDRILSQSQEVSPRTSGRLSHSSGPLNGSLTDSPPISPHQVDDIKQFCRSNNNFNSQYRSTGTTPGPITATTTQSKTVGRWLKDRREKKKEEMRAHNAQIHAAVSVAGVAAAVAAIAAATAASSSAGKDENMAKTDMAVASAATLVAAQCVEAAEVMGAERDHLASVVSSAVNVRSAGDIMTLTAGAATALRGVATLKARAMKEVWHIASVIPMDKGINLGGCSNVNGNGSYVSSSSSHSGEFLVEDNFLGHCNREWLARGGQLLKRTRKGDLHWKIVSVYINRLNQVILKMKSRHVGGTFTKKNKNVVIDVIKNVQAWPGRHLLEGGEDLRYFGLKTVPRGIVEFQCKSQREYEMWTQGVSRLIAVAAERNNRYRI

SEQ ID NO: 36 - protein, 498 aa, Arabidopsis thaliana
MERELRRPNPINSSRRPEIFSSGGSSTQLPESPRGPMEFLSRSWSVSALEVSRALHTAKSASATNRPPSSINTPIPEETLNPEKEECPPENSTSVSSQFSFAASATSQLVLERIMSQSEVSPLTSGRLSHSSGPLNGGGSFTETDSPPISPSDEFDDVIKYFRTHNTIHPLFSGTGGSRGTTGNGSNTPMAGTGPKTVGRWLKDRKEKKKEETRTQNAQVHAAVSVAAVASAVAAVAAATAASSPGKNEQMARIDMAMASAAALVAAQCVEAAEIMGADRDHLTSVVSSAVNVKSHDDIVTLTAAAATALRGAATLKARALKEVWNIAAVLPAEKGASSALCGQVDTKHSDSSFSGELPVAGEDFLGVCNQELLAKGTELLKRTRGGELHWKIVSVYINKAGQAVLKMKSKHVGGTFTKKKKHMVLEVRKDIPAWAGRDLFNGDKHHYFGLKTETKRVIEFECRNQREYEIWTQGVSRLLAIAAEKKQKSSMSKWMAP

SEQ ID NO: 37 - protein, 474 aa, Oryza sativa
MGDHHHHHHPRRGSRHAAAGDLRPPEPPLDPLEFLSRSWSASASALDAPRPPPPAPSPSAVLGIGPIAEDASSAATAACEVVDDGSAFAAAGSSFSFASAATSQLIMERILAQSEVAPLTSGRLSHSSGPLTGGGSITDSPPVSPEIDDAKYCRAASTPKPQMYRGGNKTVGRWLKDRKEKKKEETRAHNAQVHAAVSVAAVAAAVAAVAAATAAASGSGKDDRAARTDMAMASAATLVAAQCVEAAESMGAEREHLEAVIGSAVNVRTPGDIVTVTAAAATALRGAATLKARALKEVWNIAAVIPVEKGTMGGGHHHKQNMQKQHRKLESNGSSISDDLSLEEENNFLGICSQELLARGTELLKRTRKGALHWKVVSVYINRMGLVMLKMKSRHVAGTITKKKKSVVIDVCKDVAAWPGRHLLEDGEHRRYFGLRTAEHRVIEFECTSQREYEMWTKGVARLLIIASERKRAP

SEQ ID NO: 38 - protein, 480 aa, Oryza sativa
MSESGRVTAGGGRGELRPPELPRDPALEFLSRSWSASGAADVSRRALAAAAFPAAAASAAVIAEDVSGELDVDGSASGSSFSFASAATSQLIMDRIMSQSQEVSPLTSGRLSHSSGPLNGGGSLSDSPPVSPDVDDSKFCRAVSTPKPQPYRGVVGGGGKTVGRWLKERKEKKKEEARAHNAQVHAAVSVAAVAAAVAAVAAATAASGGGRDDRAARTDMAVASAATLVAAQCVEAAESLGAERDHLAAAIASAVNVRTPGDIVTITAAAATALRGAATLKARALKDVWNVAAVIPVEKNAIAAATTGGGHHKHNAQKQQHHHRHHGNGSNTSSSFSDEVAAVDDDDDDDNNFLTICSQELLARGTELLKRTRKGALHWKVVSVYIHRTGVVMLKMKSRHVAGTLTKKKKNVVVDVCRDVAAWPGRHLLEGGEHRRYFGLRTAEHRVIEFECGSQREHDMWTKGVARLLATIDGRRKRFA

SEQ ID NO: 39 - protein, 442 aa, Oryza sativa
MEQCHHHQQQSNGQQLQRLEGIEEEGGAAEKWPPPTTVRPPETPTETMEFLARSWSLSAAEISKALRVLSGKGVSDDVHDAAAAVAGDRKERRSPVTMDGRRHEQASEENEAASMRADASSMAAAAQGGAMSPPISPRANLDVKLLRATATAAAAAGGRGGGGKTTMGTWIKEQKERKRAEARSRNAQAYAATSVAGVAAAVAALVAGAVFSPPPDHPKNGGAAAPAPAPAHAAGAAGAKTAAAIASAAALVASHCVEMAQAIGASHDQILGAIQSAVNAQTSGDIMALTAGAATALRGAAMLRARLHKEIQAAALPGGGAGAGDISGREPERDTSPFAFVSRGGELLKRTRQGTITVIIKMRSAHMAGTFIKTKKFVVLDICSEIPAWAGREVEEGSHRRGYFGIKTVERMIEFECRSKYEQHKWVQGITEMLNRRHTMKN

SEQ ID NO: 40 - protein, 339 aa, Sorghum bicolor
MNFTPEIMQYCRAVSTPKPQAYRAGNKTVGRWLKDRKEKKKEETRAHNAQVHAAVSVAAVAAAVAAVAAATAAASGSGKDDRAARTDMAVASAATLVAAQCVEVAESMGAEREHLEAVVGSAVNVRTPGDIVTVTAAAATALRGAATLKARALKEVWNIAAVIPVEKGGGVGGGGGGHHQKHGAPKQQQHHHRKLESNGSSISDVSLEEENNFLGICSQELLARGTELLKRTRKGALHWKVVSVYINRMGLVMVKMKSRHVAGTITKKKKSVVIDVCRDVAAWPGRHLLEDGEHRRYFGLRTAEHRVIEFECTSQREYEMWTKGVARLLSIAAERKRLA

SEQ ID NO: 41 - protein, 422 aa, Sorghum bicolor
MEQCADHSGQLPRLEGIEEEGGPAEKWAPTTVRPPETPTETMEFLARSWSLSAAEISKALRVLSCGKAASDSPPAVATSTTEQWPAPPPPLGSDHHQHQYQQRADAAMAAGVEAGGGMSPPISPRADVKLLRAAGRGKTVGAWIKEQREKKRAEARSRNAQAYAATSVAGVAAAVAALVAGAVFSSSAAAAPEQRPAKGSGSGGAKTAAAVASAAALVASHCVEMAQAIGASHDQILAAIHSAVNAQTSGDVMALTAGAATALRGAAMLRARLHKEIQATALPGDGREPERDISPLVFVSRGGELLKRTRQGALHWKLVSVYINSTFQVVLKMQSAHMAGTFIKTKKCVVLDVCSDIPAWAGREVEEGGSHGGSRSRRGYFGIRTVERVIEFECRSRHEQRKWVQGITEMLLRRDSINNAAL

SEQ ID NO: 42 - protein, 411 aa, Sorghum bicolor
MAMEKKSSWKATRDVAAGAFSMDCLELVGEEDDDDAVPAIPPPQTPMEPMEYLSRSWSVSASEISKILVGGGGKKSSAAVAAASRLPPEVTIPELSVLATTSSSIVPLPCHHQQHRDARRSSMSSGHYHHHHQSIGKWFQVHHRETCRAKQSGKEKQRADKAHVHAMVSVARVAAAVAAVAASTATSSDSTQAATKMATAMASATELLASHCVEAAQLAGARHDQVAAAVQAAVGVRSPGDLMTLTAAAATSLRGAATLKQRVQRETRSNASVLPYEKANSWSPDIWCKEGELLKRTRKGGLHKIRVSIYINKRSQVILKLKSKHIGGALSKNNKSVVYGVYSELPTWAEPGKDSTEETCCFGLSTAQGLVEFECEGNASKQKWVDDVQNLLRQAALHDQVGNKLGLVKLS

SEQ ID NO: 43 - protein, 370 aa, Sorghum bicolor
MEFLSRSWSVSASEISKVLAAGGVGARRSSNFVVDRLSGMLVPETLALAAAASGTNISPRKRQTLRSRSAISAHQVHHTVQAIGKWFHHWDASSSKVDKARAERARVHAAVSVASVAAAVAAVAGTASAEMDDIDGARMESALASATQLLASHCVEVAELAGADHDQVASAVEAAVDVRSPGDLLTLTAAAATALRGAAAMRHRAQREARSRAAVAPYEKTGGSCRADVWCKEGALLERNRKGKHSHFCSPRALHWKQVAVYINKKSQVMVKLKSEHIGGAFSKKKKGVVYGVYDDIPAWPAHEGGGVQSSAPETCHFGLRTAQGLLEFQCERRAQRQEWVEAVKNLIRQVAGGTAQLEHSFESLRLSAS

SEQ ID NO: 44 - protein, 476 aa, Glycine max
MDKAQTEPWRPDPFHSGIFRPPETPREPMEFLSRSWSVSAHEVSKALSPALSKVTLSNGTAAVVAIPEDIAGEAEESSATVSGNPFSFASSETSQMVMERIMSQSQEVSPRTSGRLSHSSGPLNGTQSCGSLTDSPPVSPSEIDDIKYTRANNIASSLSLNFRTTATAAAPGGGKTVGRWLKDRKEKKKEETRAHNAQLHAAVSVAGVAAAIAAIAAATAATSGAGKDEQMAKTDMAVASAATLVAAQCVEAAEALGAEREHLASVVSSSVNVRSAGDIMTLTAAAATALRGAATLKARALKEVWNIAAVIPVEKNLAVAGGHNNNNNASNGNSKSSFSGELVPEENFLGICSRELLARGCELLKRTRKGDLHWKVVSVYVNRMNQVILKMKSRHVAGTITKKKKNVVLEVIKDVPAWPGRHLLEGGEDRRYFGLKTVMRGLVEFECRNQREYDVWTQGVSRLLSIAAEKNNKNRI

SEQ ID NO: 45 - protein, 486 aa, Glycine max
MEKERPARRPDPFHARPPPPPSETPRDPMEFLSRSWSASALEVSKALASQQQLPPSSNNVSIVCYNNNSNASIILEDIAGEVEESAATVSGNPFSFASSETSQMIMDRIMSHSQEVSPRTSGRLSHSSGPLNGSLTDSPPVSPSEIDDFKYNRSNNNHNTVNMITGLNGQYWGAGGAATTAGGGKTVGRWLKDRKEKKKEETRAHNAQLHAAVSVAGVAAAMAAIAAATAASSGSRKDEQMAKTDMAVASAATLVAAQCVEAAEAMGAERDHLASVVSSAVNVRSAGDITTLTAAAATALRGAATLKARVLKEVWNIAAVIPVEKNLGGGSGGGDNGNGSNGSSNSSFSGEIVPEENFLGICSRELLARGCELLKRTRTGELHWKIVSVYINRMNQVMVKMKSRHVAGTITKKKKNVVLGVIKDMPAWPGRHLLEGGENRRYFGLKTVMRGVVEFECRNQREYDVWTQGVSRLLSIAAERNNRNRI

SEQ ID NO: 46 - protein, 503 aa, Glycine max
MDKAQTEPWRPDPLHSGMFRPPETPREPMEFLSRSWSVSALEVSKALSPALSKVTLSNGAAVVAIPEDIAGEAEEASATVSGNPFSFASSETSQMVMERIMSQSQEVSPRTSGRLSHSSGPLNGTQSCGSLTDSPPVSPSEIDDIKNCLEQPRRVAPMNVLEYPNLATHSVELRPSYLAALTTLSLNFRTTAPGAAPGGAGGGKTVGRWLKDRKEKKKEETRAHNAQLHAAVSVAGVAAAVAAIAAATAATSGTGKDEEMAKTDMAVASAATLVAAQCVEAAEAMGAEREHLASVVSSAVNVRSAGDIMTLTAAAATALRGAATLKARALKEVWNIAAVIPVEKNLAAGGVNNNNASNGNSNSSFSGELVPEENFLGICSRELLARGCELLKRTRKGDLHWKVVSVYINRMNQVVLKMKSRHVAGTITKKKKNVVLEVIKDVPAWPGRHLLEGGEDRRYFGLKTVMRGVVEFECRNQREYNVWTQGVSRLLSIATEKNNKNRI

SEQ ID NO: 47 - protein, 483 aa, Glycine max
MEKERPASWRPDPFHARPPPRDPMEFLSRSWSASALEVSKALSSQQLPPSSNNNVVCYVNNSNASIILEDIAGEVEESAATVSGNPFSFASSETSQMIMDRIMSHSQEVSPRTSGRLSHSSGPLNGSLTDSPPVSPSEIDDFKYNRSNNNHNTVNMITGLNGQYWGAGGAAAAAAAGGGKTVGRWLKDRKEKKKEETRAHNAQLHAAVSVAGVAAAVAAIAAATAASSGSRKDAQMAKTDMAVASAATLVAAQCVEAAEAMGAERDHLASVVSSAVNVRSAGDITTLTAAAATALRGAATLKARVLKEVWNIAAVIPVEKNLGAGGGSNANGNRNGSNDSSNSSFSGEIVPEENFLGICSRELLARGCELLKRTRTGELHWKIVSVYINRMNQVMVKMKSRHVAGTITKKKKNVVLGVIKDMPAWPGRHLLEGGENRRYFGLKTVMRGVVEFECRNQREYDVWTQGVSRLLSIAAERSNRNRI

SEQ ID NO: 48 - protein, 488 aa, Glycine max
MEPKCTLINCRRPEFVPTPLPESPRVPMEFLSRSWSASALEVSKALAPHHSNSNNNTSSIPEETSVSAPNHNSNNSFSEDFSTTISSKNQFSFASSATSQLVLERIMSQSAREEVSPLTSGRLSHSSEPLNGGGSLTGTDSPPISPSDEFDDVVKFFRANNSIHPLFNGGRTSATIGNGTACSGPKTVGRWLKERREKKKEENRTHNAQLHATISVAAVAAAVAAIAAATAAGSSAPSKDEKMAKTDMAVASAATLVAAQCVEAAEAMGAERDHLASVVSSAVNVRSPDDITTLTAAAATALRGAATLKARALKEVWNIATVTPLERGIGGIGLCGKSINSNTSNTSTSDSGEIFNGENFLGSCSQELLAKGSELLKRTRKGDLHWKIVSVYIHRTGQVMLKMKSRHVAGTITKKKKNVVLDICTDLPAWPGRHLLGDGEKRRYFGLKTDARGIVEFECRNQREYDLWTQGVSRLLSVVAQRQNRYGN

SEQ ID NO: 49 - protein, 485 aa, Glycine max
MEPKCTINCRRPEFVPTPLPESPRVPMEFLSRSWSASALEVSKALAPHSSYNNSSSIPEQTSASAPNHNNNPFSEDLSTISSKNQFSFASSATSQLVLERIMSQSAREEVSPLTSGRLSHSSEPLNGGGSLTGTDSPPISPSDEFDDVVKFFRANNSIHPLFNGGRASATIGNGTACSGPKTVGRWLKDRREKKKEENRTHNAQLHATISVAAVAAAVAAIAAATAGSSAPSKDEKMAKTDTAVASAATLVAAQCVEAAEAMGAERDHLASVVSSAVNVRSHDDITTLTAAAATALRGAATLKARALKEVWNIATVTPLERGIGGIGLCGKSINSNTSNTSNTSTSDSGEIFKGENFPGSCSQDLLAKGSELLKRTRKGDLHWKIVSVYIHRTGQVMLKMKSRHVAGTITKKKKNVVLDICADLPAWPGRHLLDDGEKRRYFGLKTDARGIVEFECRNQREYDIWTQGVSRLLSVVAQRQSRYGN

SEQ ID NO: 50 - protein, 475 aa, Glycine max
MKCRRNENLLTSGTHLPESPRVPMEFLSRSWSASSLEVSKALTLPPPPPLSSYIPSKPPNAPSSVTNSISEETSEEFSTMCGNQFSFASSATSQLVLDRIMSQSAREEVSPLTSGRLSHSSEPLNGGASLTGTDSPPISPSDEFDDVVKFFRANNSIHPLFNGGRAMSGATGNATPCSGPKTVGRWLKDRREKKKEENRTHNAQLHASISVAAVAAAVAAVTAATAASSAADKDDKMAMAVASAATLVAAQCVEAAEAMGAERDHLASVVSSAVNVRSHDDITTLTAAAATALRGAATLKARALKEVWNITAVTPIESGIGIGICGKGNNSNSSTSDSGEIINGEIFLGACSQELLARGTALLKRTRKGDLHWKIVSVYLHRTGQVMLKMKSRHVAGTITKKKKNVVLDVCINLPAWPGRHLFDDSEERRYFGLKTEARGIVEFECRNQREYDTWTQGVSRLLSMVASRQNINGV

SEQ ID NO: 51 - protein, 466 aa, Glycine max
MEPNLTMKFRRNENLPTNGKHLPESPRVPMEFLSRSWSASSLEVSKALTPPSSISDIPSKPPNAPSSVSVTNSISEQASEEFSTMCGNQFSFASSATSQLVLDRIMSQSAREEVSPLTSGRLSHSSEPLNGGASLTGTDSPPISPSAEFDDVVKFFRANNSIHPLFNVGRAMSGATTGNATPSSGPKTVGRWLKDRREKKKEEHRAHNAQLHAAISVAAVAAAVAAVTAATAASSAANKDEKMAMAVASAATLVAAQCVEAAEAMGAERDHLASVVSSAVNVRSHDDITTLTAAAATALRGAATLKARALKEVWNITAVTPIERGIGIGICGKGNNSNSSTSDSGEIINGENFLGACSQEFLARGTALLKRTRKGQVMLKMKSRHVAGTITKKKKNVVLDVCTNLPAWPGRHLFDDSEERRYFGLKTEARGIVEFECRNQREYDTWTHGVSRLLSIVARRQNINGV

SEQ ID NO: 52 - protein, 453 aa, Arabidopsis thaliana; identical to SEQ ID NO: 3

SEQ ID NO: 53 - protein, 471 aa, Zea mays; identical to SEQ ID NO: 7

SEQ ID NO: 54 - protein, 471 aa, Zea mays; identical to SEQ ID NO: 7

SEQ ID NO: 55 - protein, 268 aa, Zea mays
MAVASAATLVAAQCVEAAEAMGAEREHLEAAVGSAVSVRTPGDIVTVTAAAATALRGAATLKARALKEVWNIAAVIPVEKGPGPVGGGGGGHHHQHHQKHGAPKQQLHRKMDSDGSSVSDLSLEEENSFLGICSQELLARGTELLKRTRKGALHWKVVSVYINRMGLVMLKTKSRHVAGTITKKKKTDERVRHCRCCRAGVVVEVCRDVPAWPGRHLLEDGGHRRYFGLRTAERRVVEFECRSQREYEMWTKGVARLLSVAAERKRLA

SEQ ID NO: 56 - protein, 469 aa, Zea mays
MGDPRARTSAVAAGELRPPEPALDPLEFLSRSWSASGRALAPTPPPPPAAFAASPIAEDAACELDDGGVVAAASGSSFSFASAATSQLIMERILAQSQEVAPLTSGRLSHSSGPLNGGGSLSDSPPVSPEIDDAKYCRAASTPKAQAYRAGNKTVGRWLKDRREKRKEETRAHNAQVHAAVSVAAVAAAVAAVAAATATASASGGKDERAARTDMAVASAATLVAAQCVEAAEAMGAEREHLEAAVGSAVSVRTPGDIVTVTAAAATALRGAATLKARALKEVWNIAAVIPVEKGPGPVGGGGGGHHHQHHQKHGAPKQQLHRKMDSDGSSVSDLSLEEENSFLGICSQELLARGTELLKRTRKGALHWKVVSVYINRMGLVMLKTKSRHVAGTITKKKKSVVVEVCRDVPAWPGRHLLEDGGHRRYFGLRTAERRVVEFECRSQREYEMWTKGVARLLSVAAERKRLA

SEQ ID NO: 57 - protein, 255 aa, Zea mays
MAVASAATLVAAQCVEAAEALGAEREQLAAAVASAVSVRTPSDVATVTAAAATALRGAATLRARALKEAWNVAAVTPVEKGAMTGGGGHHLHGGGYKHDGQQQRQRLLREVESSNSSSSCLSDELVLGEENNFLGICTQELLARGTELLKRTRKGSLHWKVVSVYINRAGLVMLKMKSRHVGGTITKKKKSVVVDVCRDVAAWPGRHLLDSGEHRRYFGLRTAEQRVIEFECGSQREHEVWTKGVARLLADARKR

SEQ ID NO: 58 - protein, 472 aa, Setaria italica
PPEPPRDPLEFLSRSWSASAADVSRALAAAPAPALVPIAGAGAAIDEDVAGELDCDGSGACGGGGPASGSSFSFASAATSQLIMDRIMAQSQEVSPLTSGRLSHSSGPLNGGGSLSDSPPVSPEIDEAKFCRLASTPKPQPYSRGGSKTVGRWLKDRRERKKEETRAHNAQVHAAVSVAAVAAAVAAVAAATAAASSGSGKDDRGARTDMAVASAATLVAAQCVEAAEAMGAEREHLAAAVGSAVNVRNPGDVVTITAAAATALRGAATLKARVLKEVWNGAAVIPVEKGAMAGGGGGRHHQQSHKHNGQLKHQHQHQLRQRELESSNSSSSCFSDELLLAEENNFLGICTQELLARGSELLKRTRKGSLHWKVVSVYINRMGLVMLKMKSRHVGGTITKKKKSVVVDVCRDVAAWPGRHLLEGGEHRRYFGLRTAEHRVIEFECGSQREHDMWTKGVARLLAIADARKRAV

SEQ ID NO: 59 - protein, 411 aa, Zea mays; identical to SEQ ID NO: 13

SEQ ID NO: 60 - protein, 422 aa, Sorghum bicolor; identical to SEQ ID NO: 41

SEQ ID NO: 61 - protein, 427 aa, Zea mays; identical to SEQ ID NO: 15

SEQ ID NO: 62 - protein, 429 aa, Zea mays
MQHEEVCDTTTCGEGNGRGGEEELEMEGHSLMRMKRRDHLLLTDGAGAGAGAGPLAVQTPMEPMEFLSRSWSVSASEISKVLAGGVGGRRSSNFVVDRLSGMLMPETLALAASSSSAGTNNISPRKRTLRSRSAISAHQVHHTVHAIGKWFHHWDAGSSKVDKARAERARVHAAVSVASVAAAVAAVAVAAGASPEVDVTDDGARMGSALASATQLLASHCVEAAELAGADHDQVASAVETAVDVRSPGDLLTLTAAAATALRGAAAMRHRAQREARSRAAVAPYDKACSYRADVWCKEGALLKRNRKGKRALHWKQVAVYISKKSQVIVKLKSKHIGGAFSKKKKGVVYGVYDDIPAWPAREGVAAQSSAPETCHFGLRTAHGLLEFQCESRAQRRDWVEAVKDLIRQVAGGTALLEHSFESLRLSAS

SEQ ID NO: 63 - protein, 662 aa, Artificial Sequence
  consensus sequence for the alignment figure
MXXXXXXXXXXXXXXXXXXXXXXXXXXXXDXXRXXXXXXXXXXXXXXXPPXXXPXXPETPLEPMEFLSRSWSASAXEVSKALAPXXXXXXXXXXXXXXXXXXXXXXXXXXXXAGXXXXXXXXXXXXXAAASGNXFSFASSATSQLVMERIMAQSXXQEVSPXTSGRLSHSSGPLNXXXXGGXXXXDSPPVSPXXEIDDXKYCXXXXXXXXXXXXXXXXXXXRAXXXXXXXXXXXXXXRGGGKTVGRWLKDRREXXKKKEETRAHNAQVHAAVSVAGVAAAVAAVAAATXXXXXXXXXAAASXSGXKDERXARTDMAVASAATLVAAQCVEAAEAMGAEREHLAXXVXSAVNVRTPGDIMTLTAAAATALRGAATLKARALKEVWNIAAVIPVEKGAXXXGXXXXXXXXXXXXXXXXXXXXXXXXXXXXXXXXXSSXXXXXXXXXLXEENNFLGICSQELLARGTELLKRTRKGXXALHWKVVSVYINRMGXXXXXXXXXXXXXXXXXXXXXXXXXXXXXXQVMLKMKSRHVAGTITKKKKXXXXXXXXXXXXXXXXXXXXXXXXXXXXXXXXXVVVDVCXDXPAWPGRHLLEGGEHXXXXXXRRYFGXXXLRTAERRVIEFECRSQREYEMWTXGVARLLSIAXXXXXXXXRXDERXRLXAX

SEQ ID NO: 64 - DNA, 3109 bp, Arabidopsis thaliana
atcctatacaccacccagaaccattcacactaatccttggtgcatttttccttatgtgaacccacagagccaaaactttctttttccagagccgaatgagatcatatcacaacgactatattctgatcatcttttgcgggtcttgaagattcctacaattaatgatgcctaagttatcaagactcaagacacatcacaactttcctagtatggccgattctctcattcaattttcaacggccatattttcatttattgttgcgatttctttctttattttagcatatagacgttgcacttgaacccctatataacatagtcagacgtttcgggttcctatatgcgagccgcaagagatgaccaagacggacgaaggatacccacatggctttggagtcaagaactcactggtcatgcttggtgtcattaactttgtcggtattctcttcaccttccttgttccttagcccaagggcagagccggccatgagcacaagcgggccaagcccatgcttgcagccgattattcaataattacattttcggcctccaaatactgaagttgttctatggtccaatgttcgcgtcagggaaaacttggtccttgttttgtttaacttcttaagttcgaatctcccaaatgccgatattattacagtttttcttcttccgtttttgtcaaaaaaaaattagttactaaattataaataattaacctcaattgcaattaatcttttggttccttttcttcctctagcattgtgtggtttattggtccttaaactgtcagctttttagttcaaatccatccattttgtctattttgtctctgtatctcatacttattctataattcattagtggtttaactgtaacaaattcatgttttattctgataagttttttttctttacttcttaatcattgtttcttcataaaaacaacatatgaaggatatgatagaaatttgactaaaaaaaacttgatggatgagtctacgtacatatataaatgcaaggaatatcatatataaaaattgcattgatctttgatgctttaatacttttagtgctttttttaaacataaccaattttatattttgtttgatcacacttttttatactatgaacatgtgtaaagaggctttttttatattatgaacatgtgtaaaatatcctctcttttttttttactataagcatgtgttaagtggccttttctttttctttgcttgtagccttgtaaataataggaatgcccaagggcaagtcccttgaagagctctccggtgaggctgaggttgagaaatgattacgccataatattttcaaattcttaattggttcgatgaattacatcttctattacgatttactttatttttgcgatgcgtaatgtgatatttcatgttcgtaatggtggatctctacatttatttttttgtgtgacaaaatgtaatcaatcgatatttagtggtcatttatgatatttatttgactgcttatctctactacacagctactatcaaattcctaatctttgttgttattatggttatatttaatttatgcatgtaatacctctcgtataacaacaaaacattcaaaccatttaaaacattaacgtgtacatatcgaagtccagaaaacccgtcatttaaaacaaatacaatcctcacatttatatatatatatggtctgagctggattggttgatattaaatccttatttatgtatatgtaatggcaactaagtccactaaggcatgaattatctgttaaaatgaatttatatgcttatgttctcgcgaatatcctatgtttttcgaatatgcttttttaaaataatgattttgactttgctaaatgataattaatcttcatgactatatgtatcctgattgtcactcctcaaatgtcaactattagagttgaataactcgactgcaaaaaacaaaacaaagaaaacgaggaagatggagaaatttaaaacctttcgacttatattggctctaaaataacttgtagttcagatttacacgaaacaataaataaatgattagagcttgcaactctccttatttaattttagaaatgcattttatattgcgaataatataattgcaaagttggaatcaaacataaatatatataactggtcaacgattacaagtaatgccaacaagattaactacaaactgaattcgtgtctacactacttctttttgattttattttattctttttttgtgtgtgtacaacgtctaaattagctacttctatttatagaaatttacatggaatgcatatctatagtaatgctcatatatcaatctgtctccatatagtaattgagatggttaaacagaaatttttgctaaaacttcgtttcctgcttcaaaataataatcagaatccgtaactatcatacaataaaaaaatcatcccatctcacgtatcctagagagatttattgtataaggtgttcatattctggaaggcgtatcgtccagcttagatgatgttttatacttttcaatataaaatagactgtaaataatattttacttcagaaacaaatggtgttcaaaaatcttaaacatcaactataaacatacggagggagtattgatgactaaaattctaaaatccaaaatgaaattacaaaataaataataactatgaagaaagaggcgatcacgttgcttacatatctttccttttcaaaaagcatttttttattacatttaacgtgcttctctttcttcttctctataaatggccagcataattgttcatttctaatgaccacatatagtctctcctcaactctccagagaagttcttaatttctcctgccaagctgattaaggtaatattactttcctttgtcgtctatatttaatttgtgtatataccatttcaagttgagttttcttgttcataattccaaagatatcaagttttgtttcaaaattttggccattgagaggtcaaatgctctgttccatgatattttttaagtctccaaatgaaaatatttcataagcgagaaataatgacatgaaatcttggattctttgcgttgtatgattatagagctctagga

SEQ ID NO: 65 - DNA, 1595 bp, Arabidopsis thaliana
gaatgagcaaacattgccgaagttagctagcaacttggacatggggaacatggaagagaacacgccttctgatctggccaatgggaatgagaaaacagagcctaactcagccaacagcatggatttagggaacatggaagagaacacgccttctgatttggcaaatgggaataagaaaaaagagcctaagtcagtcagcaagttggatctgggtagcgagaaagtgagcattccttctaatctcgtcaatggaaatgagcaacatgacctaaacattgctccaggtgaagatgcttcagcggctaagcaaccagaaaagaagcgatccaggtctaaaaagcgcaaatcaggcaacaacctggatttggggaagatggaaaagagcaagccttccgatctggcaaatgagaatgagcaaacagagcccaagtcagccaacaacttggatttagggaatatgaaagagaacacgccttcggatctggctaatgagaatgagcaaacagagcttaggctacctaacaactcggattatggtaacatggaagagagcttgcctttgaatcttgctaatggggatgaacaacctgacccaaccactgctccaatggaagctgctaagcaaccaaaaaagaagcgatcaaggtccaaaaagtgcaaatcagtcaacaacctggacttggggaacatggaagaaaacaagccttctgatctggcaaacggaaatgagcaaaaagatcctgagtcagtcaacaggttggatccgggtaaggagaaagagagcattccttcgaatcttgtcagtgggaatgagcaacctgactcaaacactgctccagctaagaaaccaaaaaagaaaaagcgcaaattagctaacaactttgattcggttaacaatatggaagagaaaatgccttccactaatgtattgagccagggaaacaagtcaggtttgattgaggaaaaaccgagcataccttccgatcaatgagcagtgtggctcaaatactgcaccaacctaagcaaaaccgaaagaagaagggaagtatgatgcccattccactttggtgttttaatgagcatttcggtgtttgtcatgaaaccaagttttgaaatggcacttgtctacataagtgtaacagattcttaatttcaacaattttgttagataatttgtattcattctcacttagtgcttccgttgttctatcgttataaacgttttaagatatacgcatgtaactgaaagaagaaaagccattattgatccagtactgtagtgtcttagtgtgagcagaaaaaagatttgatcatggaatggctttagagtaaaatactaaaatagatggaatcgaaggcttatgaatctgacgtggaacaagaagaaagcgtatctgccacgtggcacaatacaacacctcaacagccttatctggacatcaacagagagatcctccaatatccgaaaccaaccaatcagcttcactcttggtttcacctccatacattaacacaccaacaatcctcaaaaaacactttcataacttaatcacacacaaggagagaagagagaaagagagaaagagagacagaga

SEQ ID NO: 66 - DNA, 2998 bp, Arabidopsis thaliana
actattgcttgatccgcttggcccattggacgactttgtttgtaaatgtccgaatattttttcaagatatttatgtatttggtacttcaaatctagtacccaagccttagatcattaaaatttagaactcagtatataactaatgccattttttatttttggattcgatatttttttttttagtgtaatattaaattatatttaaaaaaacaaatatacgtttttataatttgtgcttcgtatgtttgaaaaagtaagctctaaagatttctgatgatttttcgaaaatgtttaaagaaaatgtttaaacaagctaagagtcgaggttaactagtcatgggagtgaatccagttttaaataggcatcgtcatactgcttgtcgcctaataatcgtattgctgaaatcagatttcgaatttgtttatcaacccatatggtgataccctttatattatgatcccacgcacctcaaaaatttcggcgttggattcatgtcactcctagctagcaacctcagggctcgcagtatcccattcgtcttaattagttaagattttttttaaaaatttattttgttcagttattattcatttcattcgaacgattaaacaaagattattatggtctcgtgatttatatgttgacccttctaatattctaaatttaaaaataagaaaaaagaggaaaattgaggcaaaacacatgttgccgagtcattatcaactaggggtgcataatgatggaacaaagcacaaatcttttaacgcaaactaactacaaccttcttttggggtccccatccccgaccctaatgttttggaattaataaaactacaatcacttaccaaaaaataaaagttcaaggccactataatttctcatatgaacctacatttataaataaaatctggtttcatattaatttcacacaccaagttactttctattattaactgttataatggaccatgaaatcatttgcatatgaactgcaatgatacataatccactttgttttgtgggagacatttaccagatttcggtaaattggtattcccccttttatgtgattggtcattgatcattgttagtggccagacatttgaactcccgtttttttgtctataagaattcggaaacatatagtatcctttgaaaacggagaaacaaataacaatgtggacaaactagatataatttcaacacaagactatgggaatgattttacccactaattataatccgatcacaaggtttcaacgaactagttttccagatatcaaccaaatttactttggaattaaactaacttaaaactaattggttgttcgtaaatggtgctttttttttttgcggatgttagtaaagggttttatgtattttatattattagttatctgttttcagtgttatgttgtctcatccataaagtttatatgttttttctttgctctataacttatatatatatatgagtttacagttatatttatacatttcagatacttgatcggcattttttttggtaaaaaatatatgcatgaaaaactcaagtgtttcttttttaaggaatttttaaatggtgattatatgaatataatcatatgtatatccgtatatatatgtagccagatagttaattatttgggggatatttgaattattaatgttataatattctttcttttgactcgtctggttaaattaaagaacaaaaaaaacacatacttttactgttttaaaaggttaaattaacataatttattgattacaagtgtcaagtccatgacattgcatgtaggttcgagacttcagagataacggaagagatcgataattgtgatcgtaacatccagatatgtatgtttaattttcatttagatgtggatcagagaagataagtcaaactgtcttcataatttaagacaacctcttttaatattttcccaaaacatgttttatgtaactactttgcttatgtgattgcctgaggatactattattctctgtctttattctcttcacaccacatttaaatagtttaagagcatagaaattaattattttcaaaaaggtgattatatgcatgcaaaatagcacaccatttatgtttatattttcaaattatttaatacatttcaatatttcataagtgtgattttttttttttttgtcaatttcataagtgtgatttgtcatttgtattaaacaattgtatcgcgcagtacaaataaacagtgggagaggtgaaaatgcagttataaaactgtccaataatttactaacacatttaaatatctaaaaagagtgtttcaaaaaaaattcttttgaaataagaaaagtgatagatatttttacgctttcgtctgaaaataaaacaataatagtttattagaaaaatgttatcaccgaaaattattctagtgccactcgctcggatcgaaattcgaaagttatattctttctctttacctaatataaaaatcacaagaaaaatcaatccgaatatatctatcaacatagtatatgcccttacatattgtttctgacttttctctatccgaatttctcgcttcatggtttttttttaacatattctcatttaattttcattactattatataactaaaagatggaaataaaataaagtgtctttgagaatcgaacgtccatatcagtaagatagtttgtgtgaaggtaaaatctaaaagatttaagttccaaaaacagaaaataatatattacgctaaaaaagaagaaaataattaaatacaaaacagaaaaaaataatatacgacagacacgtgtcacgaagataccctacgctatagacacagctctgttttctcttttctatgcctcaaggctctcttaacttcactgtctcctcttcggataatcctatccttctcttcctataaatacctctccactcttcctcttcctccaccactacaaccaccgcaacaaccaccaaaaaccctctcaaagaaatt

SEQ ID NO: 67 - DNA, 55 bp, Artificial Sequence
  At2g36030-5′attB primer
ttaaacaagtttgtacaaaaaagcaggctcaacaatgacggtggacgagtggaga

SEQ ID NO: 68 - DNA, 50 bp, Artificial Sequence
  at2g36030-3′attB reverse primer
ttaaaccactttgtacaagaaagctgggtctagatttccgacagtccatg

SEQ ID NO: 69 - DNA, 213 bp, Arabidopsis thaliana
atgacggtggacgagtggagactcttggacgtctccacggcgtttggcagcggagaaaactccgtcccagatttcggagaaagcgcggatgatgatgccgtggtggtatggctcgttaagggagaggaagcagtggagaagctctctgagatcgtttttggagtaaatctcgttctcgagaatcatctgaagcatggactgtcggaaatctag

SEQ ID NO: 70 - protein, 70 aa, Arabidopsis thaliana
MTVDEWRLLDVSTAFGSGENSVPDFGESADDDAVVVWLVKGEEAVEKLSEIVFGVNLVLENHLKHGLSEI

SEQ ID NO: 71 - DNA, 213 bp, Artificial Sequence
  maize optimized NUCPU29 sequence
atgactgttgacgagtggagattgctcgacgtgtctacagccttcggcagcggcgagaactccgtgccagacttcggcgagtctgccgacgacgacgccgttgttgtgtggttggtgaagggtgaagaggccgtggagaagctgtccgagatcgtgttcggcgtgaaccttgtcttggagaatcacctcaagcacggcctgtcagagatctag

SEQ ID NO: 72 - DNA, 25 bp, Artificial Sequence
  attB1 site
acaagtttgtacaaaaaagcaggct

SEQ ID NO: 73 - DNA, 25 bp, Artificial Sequence
  attB2 site
accactttgtacaagaaagctgggt